SEQUENCE LISTING (180 sequences)

SEQ ID NO: 1 - DNA, 438 bp, synthetic construct
  source 1..438: mol_type = other DNA
atgccacccataaggaatgtaacagttgcttacaagttccacatgggtctgnnknnkgaaaccggtcgtttgnnknnkgaaagcnnkagcatcagcccgaaactgcgcagcatcgccgtgtattacgacaacccgcatatggttcctccggataagtgccgttgtgcagttggtagcatcctctccgaaggcgaggagtccccatctccggagctgattgacctgtaccagaaattcggcttcaaggtgttcagctttccggcaccgtcacatgtcgtgactgcgacgtttccgtataccaccnnkttgtctnnknnkctggctacccgtcgtgttcacccggcgctggatacctacatcaaagagcgcaaactttgcgcgtatccgcgtttggaaatttatcaagaggaccagattcactttatgtgcccgctggcg

SEQ ID NO: 2 - protein, 146 aa, synthetic construct
MPPIRNVTVAYKFHMGLXXETGRLXXESXSISPKLRSIAVYYDNPHMVPPDKCRCAVGSILSEGEESPSPELIDLYQKFGFKVFSFPAPSHVVTATFPYTTXLSXXLATRRVHPALDTYIKERKLCAYPRLEIYQEDQIHFMCPLA

SEQ ID NO: 3 - DNA, 438 bp, synthetic construct
  source 1..438: mol_type = other DNA
atgccacccataaggaatgtaacagttgcttacaagttccacatgggtctgdmyvrngaaaccvrncgtttgdmywbggaaagcvrnagcatcagcccgaaactgcgcagcatcgccvrntattacgacaacccgvrnatggttcctccggataagtgccgttgtgcagttggtagcatcctctccgaaggcgaggagtccccatctccggagctgattgacctgtaccagaaattcggcttcaaggtgttcagctttccggcaccgtcacatgtcgtgactgcgacgtttccgtataccaccvrnttgtctvrndmyctggctacccgtcgtgttcacccggcgctggatacctacatcaaagagcgcaaactttgcgcgtatccgcgtttggaaatttatcaavrngaccagattcactttatgtgcccgctggcg

SEQ ID NO: 4 - protein, 146 aa, synthetic construct
MPPIRNVTVAYKFHMGLXXETXRLXXESXSISPKLRSIAXYYDNPXMVPPDKCRCAVGSILSEGEESPSPELIDLYQKFGFKVFSFPAPSHVVTATFPYTTXLSXXLATRRVHPALDTYIKERKLCAYPRLEIYQXDQIHFMCPLA

SEQ ID NO: 5 - DNA, 480 bp, synthetic construct
  source 1..480: mol_type = other DNA
atgagcggtctgctcgctggcgtggaggttagcgcaggctccccacccataaggaatgtaacagttgcttacaagttccacatgggtctgnnknnkgaaaccnnkcgtttgnnknnkgaaagcnnkagcatcagcccgaaactgcgcagcatcgccnnktattacgacaacccgnnkatggttcctccggataagtgccgttgtgcagttggtagcatcctctccgaaggcgaggagtccccatctccggagctgattgacctgtaccagaaattcggcttcaaggtgttcagctttccggcaccgtcacatgtcgtgactgcgacgtttccgtataccaccnnkttgtctnnknnkctggctacccgtcgtgttcacccggcgctggatacctacatcaaagagcgcaaactttgcgcgtatccgcgtttggaaatttatcaannkgaccagattcactttatgtgcccgctggcgtaa

SEQ ID NO: 6 - protein, 159 aa, synthetic construct
MSGLLAGVEVSAGSPPIRNVTVAYKFHMGLXXETXRLXXESXSISPKLRSIAXYYDNPXMVPPDKCRCAVGSILSEGEESPSPELIDLYQKFGFKVFSFPAPSHVVTATFPYTTXLSXXLATRRVHPALDTYIKERKLCAYPRLEIYQXDQIHFMCPLA

SEQ ID NO: 7 - DNA, 564 bp, synthetic construct
  source 1..564: mol_type = other DNA
atgggttcttctgttgaagttcacccggttctggaaaaactgaaagcgattaataactacaacccgaaagattttgattggaacctgaaaaatggccgtgtgttcattattaaaagcnnkagcgaagatgatatccaccgcagcattaaatatagcattnnknnkagcnnkgaacatggcaacaaacgtctggatgcggcgtatcgcagcctgaacggcaaaggtccgctgtacctgctgtttagcgtgnnkggctcgggtcacttttgcggcgtggccgaaatgaaaagcgtggtggattacaacgcctatgcgggtgtgnnkagccaggataaannkaaaggcaaattcgaagtgaaatggattttcgtaaaagatgtgccgnnkaatcagctgcgccatattcgcctggaaaataacgataacaaaccggtgaccnnkagcnnknnkacccaggaagtgccgctggaaaaagccaaacaggtgctgaaaattattgcgacctttaaacataccacctcgattttcgatgattttgcgcattatgaaaaacgccaggaataa

SEQ ID NO: 8 - protein, 187 aa, synthetic construct
MGSSVEVHPVLEKLKAINNYNPKDFDWNLKNGRVFIIKSXSEDDIHRSIKYSIXXSXEHGNKRLDAAYRSLNGKGPLYLLFSVXGSGHFCGVAEMKSVVDYNAYAGVXSQDKXKGKFEVKWIFVKDVPXNQLRHIRLENNDNKPVTXSXXTQEVPLEKAKQVLKIIATFKHTTSIFDDFAHYEKRQE

SEQ ID NO: 9 - DNA, 1053 bp, synthetic construct
  source 1..1053: mol_type = other DNA
atgggccgcagccgcctgctggaagattttcgcaacaaccgctatccgaacctgcagctgcgcgaaattgcgggccatattatggaatttagccaggatcagcatggcmrhbkctttattvrvctgaaactggaacgcgcgaccccggcggaacgccagctggtgtttaacgaaattctgcaggcggcgtatcagctgatggtggatgtgtttggcddcydcgtgattcmaaaattttttgaatttggcagcctggaacagaaactggcgctggcggaacgcattcgcggccatgtgctgagcctggcgctgcagatgtatggchvbhncgtgattsraaaagcgctggaatttattccgagcgatcagcagaacgaaatggtgcgcgaactggatggccatgtgctgaaatgcgtgaaagatcagaacggcavhndsgtggtgyvcaaatgcattgaatgcgtgcagccgcagagcctgcagtttattattgatgcgtttaaaggccaggtgtttgcgctgagcacccatccgtatggcvvavnmgtgattvrhcgcattctggaacattgcctgccggatcagaccctgccgattctggaagaactgcatcagcataccgaacagctggtgcaggatcagtatggcvncydbgtgattvrvcatgtgctggaacatggccgcccggaagataaaagcaaaattgtggcggaaattcgcggcaacgtgctggtgctgagccagcataaatttgcgrdshnbgtggtgvrmaaatgcgtgacccatgcgagccgcaccgaacgcgcggtgctgattgatgaagtgtgcaccatgaacgatggcccgcatagcgcgctgtataccatgatgaaagatcagtatgcgmamywcgtggtgcvaaaaatgattgatgtggcggaaccgggccagcgcaaaattgtgatgcataaaattcgcccgcatattgcgaccctgcgcaaatatacctatggcaaacatattctggcgaaactggaaaaatattatatgaaaaacggcgtggatctgggctaa

SEQ ID NO: 10 - protein, 350 aa, synthetic construct
MGRSRLLEDFRNNRYPNLQLREIAGHIMEFSQDQHGXXFIXLKLERATPAERQLVFNEILQAAYQLMVDVFGXXVIXKFFEFGSLEQKLALAERIRGHVLSLALQMYGXXVIXKALEFIPSDQQNEMVRELDGHVLKCVKDQNGXXVVXKCIECVQPQSLQFIIDAFKGQVFALSTHPYGXXVIXRILEHCLPDQTLPILEELHQHTEQLVQDQYGXXVIXHVLEHGRPEDKSKIVAEIRGNVLVLSQHKFAXXVVXKCVTHASRTERAVLIDEVCTMNDGPHSALYTMMKDQYAXXVVXKMIDVAEPGQRKIVMHKIRPHIATLRKYTYGKHILAKLEKYYMKNGVDLG

SEQ ID NO: 11 - DNA, 477 bp, synthetic construct
  source 1..477: mol_type = other DNA
atggatctgggcgaactgctgctggaagcggcgcaggcgggccagctggatgaagtgcatattctgatggcgaatggcgcggatgtgaatgcgnnkgatnnknnkggcnnkaccccgctgcatctggcggcgnnknnkggccatctggaaattgtggaagtgctgctgaaahayggcgcggatgtgaatgcgnnkgatnnknnkggcnnkaccccgctgcatctggcggcgnnknnkggccatctggaaattgtggaagtgctgctgaaahayggcgcggatgtgaatgcgnnkgatnnknnkggcnnkaccccgctgcatctggcggcgnnknnkggccatctggaaattgtggaagtgctgctgaaahayggcgcggatgtgaacgcgcaggataaatttggcaaaaccccgtttgatctggcgattgataatggcaacgaagatattgcggaagtgctgcagaaagcggcgtaa

SEQ ID NO: 12 - protein, 158 aa, synthetic construct
MDLGELLLEAAQAGQLDEVHILMANGADVNAXDXXGXTPLHLAAXXGHLEIVEVLLKXGADVNAXDXXGXTPLHLAAXXGHLEIVEVLLKXGADVNAXDXXGXTPLHLAAXXGHLEIVEVLLKXGADVNAQDKFGKTPFDLAIDNGNEDIAEVLQKAA

SEQ ID NO: 13 - DNA, 714 bp, synthetic construct
  source 1..714: mol_type = other DNA
atgaccaacaaagaactgcaagccatacgtaaacttttgatgttggacgtgagtgaggcggcagagcacataggccgcgtgdmcgctsrvdmctggsrvbrctgggaatctggtsrvtcggcggtgccggacsrvgtagagcaagagatgcttgacctcgcttcagttcggatcgagatgatgtccgcgatcgataagcgtttagcagatggcgagcggccaaaacttcgcttttacaacaagttagacgagtacttagcagacaatccggaccataacgttataggttggcgtttatcacaatcggtcgccgctctctattatacagagggacacgcggacctgataggtggcggtggtagtatgactaataaagaactgcaagccattcggaagttacttatgcttgacgtttctgaagcggcggagcacattgggagagttdmcgccsrvdmctggsrvbrctgggagtctggcsrvagcgcggttcccgacsrvgttgagcaagagatgttagatcttgctagtgtgcgtatcgaaatgatgagcgctatcgacaagcggttagctgacggagaacgtccaaagttgcgcttctataacaaactggacgagtacttggctgacaaccctgatcacaatgttattgggtggcggctttcacagagtgtggcggcgttatactataccgaggggcacgctgaccttatttaa

SEQ ID NO: 14 - protein, 237 aa, synthetic construct
MTNKELQAIRKLLMLDVSEAAEHIGRVXAXXWXXWESGXSAVPDXVEQEMLDLASVRIEMMSAIDKRLADGERPKLRFYNKLDEYLADNPDHNVIGWRLSQSVAALYYTEGHADLIGGGGSMTNKELQAIRKLLMLDVSEAAEHIGRVXAXXWXXWESGXSAVPDXVEQEMLDLASVRIEMMSAIDKRLADGERPKLRFYNKLDEYLADNPDHNVIGWRLSQSVAALYYTEGHADLI

SEQ ID NO: 15 - protein, 146 aa, synthetic construct
MPPIRNVTVAYKFHMGLYGETGRLFTESCSISPKLRSIAVYYDNPHMVPPDKCRCAVGSILSEGEESPSPELIDLYQKFGFKVFSFPAPSHVVTATFPYTTILSIWLATRRVHPALDTYIKERKLCAYPRLEIYQEDQIHFMCPLA

SEQ ID NO: 16 - protein, 146 aa, synthetic construct
MPPIRNVTVAYKFHMGLNGETNRLNMESRSISPKLRSIAHYYDNPNMVPPDKCRCAVGSILSEGEESPSPELIDLYQKFGFKVFSFPAPSHVVTATFPYTTGLSMDLATRRVHPALDTYIKERKLCAYPRLEIYQDDQIHFMCPLA

SEQ ID NO: 17 - protein, 146 aa, synthetic construct
MPRIRNVTVAYKFHMGLNRETGRLSLESNSISPKLRSIAGYYDNPGMVPPDKCRCAVGSILSEGEESPSPELIDLYQKFGFKVFSFPAPSHVVTATFPYTTELSRDLATRRVHPALDTYIKERKLCAYPRLEIYQRDQIHFMCPLA

SEQ ID NO: 18 - protein, 122 aa, synthetic construct
MQVQLVESGGGSVQAGGSLRLSCTASGXXXXXYXXFXLGWFRQAPGQEREAVAAIXXXGGXTYYADSVKGRFTISRDNAKNTVTLQMNNLKPVDTAIYYCAAXXXXXXXXXXWGQGTQVTVS

SEQ ID NO: 19 - protein, 122 aa, synthetic construct
MQVQLVESGGGSVQAGGSLRLSCTASGTGGFGYNAFSLGWFRQAPGQEREAVAAISLRGGITYYADSVKGRFTISRDNAKNTVTLQMNNLKPVDTAIYYCAARDYSNGTFQSWGQGTQVTVS

SEQ ID NO: 20 - protein, 146 aa, synthetic construct; identical to SEQ ID NO: 15

SEQ ID NO: 21 - protein, 146 aa, synthetic construct
MPPIRNVTVAYKFHMGLYGETRRLYSESRSISPKLRSIAGYYDNPRMVPPDKCRCAVGSILSEGEESPSPELIDLYQKFGFKVFSFPAPSHVVTATFPYTTELSGALATRRVHPSLDTYIKERKLCAYPRLEIYQRDQIHFMCPLA

SEQ ID NO: 22 - protein, 146 aa, synthetic construct
MPPIRNVTVAYKFHMGLASETGRLASESGSISPKLRSIASYYDNPSMVPPDKCRCAVGSILSEGEESPSPELIDLYQKFGFKVFSFPAPSHVVTATFPYTTQLSRSLATRRVHPALDTYIKERKLCAYPRLEIYQNDQIHFMCPLA

SEQ ID NO: 23 - protein, 146 aa, synthetic construct; identical to SEQ ID NO: 15

SEQ ID NO: 24 - protein, 146 aa, synthetic construct
MPPIRNVTVAYKFHMGLSEETSRLNRESRSISPKLRSIADYYDNPKMVPPDKCRCAVGSILSEGEESPSPELIDLYQKFGFKVFSFPAPSHVVTATFPYTTQLSNALATRRVHPALDTYIKERKLCAYPRLEIYQRDQIHFMCPLA

SEQ ID NO: 25 - protein, 146 aa, synthetic construct
MPPIRNVTVAYKFHMGLSRETDRLYSESRSISPKLRSIAGYYDNPQMVPPDKCRCAVGSILSEGEESPSPELIDLYQKFGFKVFSFPAPSHVVTATFPYTTSLSGYLATRRVHPALDTYIKERKLCAYPRLEIYQQDQIHFMCPLA

SEQ ID NO: 26 - protein, 146 aa, synthetic construct; identical to SEQ ID NO: 15

SEQ ID NO: 27 - protein, 146 aa, synthetic construct
MPPIRNVTVAYKFHMGLWMETGRLARESVSISPKLRSIAVYYDNPHMVPPDKCRCAVGSILSEGEESPSPELIDLYQKFGFKVFSFPAPSHVVTATFPYTTMLSTLLATRRVHPALDTYIKERKLCAYPRLEIYQNDQIHFMCPLA

SEQ ID NO: 28 - protein, 146 aa, synthetic construct
MPPIRNVTVAYKFHMGLDHETRRLYRESDSISPKLRSIASYYDNPNMVPPDKCRCAVGSILSEGEESPSPELIDLYQKFGFKVFSFPAPSHVVTATFPYTTGLSEDLATRRVHPALDTYIKERKLCAYPRLEIYQNDQIHFMCPLA

SEQ ID NO: 29 - protein, 146 aa, synthetic construct; identical to SEQ ID NO: 15

SEQ ID NO: 30 - protein, 146 aa, synthetic construct; identical to SEQ ID NO: 16

SEQ ID NO: 31 - protein, 146 aa, synthetic construct; identical to SEQ ID NO: 17

SEQ ID NO: 32 - protein, 145 aa, synthetic construct
MPPIRNVTVAYKFHMGLYGETRRLYSESRSISPKLRSIAGYYDNPRMVPPDKCRCAVGSILSEGEESPSPELIDLYQKFGFKVFSFPAPSHVVTATFPYTTELSGALATRVHPSLDTYIKERKLCAYPRLEIYQRDQIHFMCPLA

SEQ ID NO: 33 - protein, 145 aa, synthetic construct
MPPIRNVTVAYKFHMGLASETGRLASESGSISPKLRSIASYYDNPSMVPPDKCRCAVGSILSEGEESPSPELIDLYQKFGFKVFSFPAPSHVVTATFPYTTQLSRSLATRVHPALDTYIKERKLCAYPRLEIYQNDQIHFMCPLA

SEQ ID NO: 34 - protein, 146 aa, synthetic construct; identical to SEQ ID NO: 24

SEQ ID NO: 35 - protein, 146 aa, synthetic construct
MPPIRNVTVAYKFHMGLSRETRRLATESGSISPKLRSIASYYDNPRMVPPDKCRCAVGSILSEGEESPSPELIDLYQKFGFKVFSFPAPSHVVTATFPYTTRLSNALATRRVHPALDTYIKERKLCAYPRLEIYQDDQIHFMCPLA

SEQ ID NO: 36 - protein, 146 aa, synthetic construct
MPPIRNVTVAYKFHMGLSGETDRLTWESESISPKLRSIAQYYDNPQMVPPDKCRCAVGSILSEGEESPSPELIDLYQKFGFKVFSFPAPSHVVTATFPYTTKLSDDLATRRVHPALDTYIKERKLCAYPRLEIYQRDQIHFMCPLA

SEQ ID NO: 37 - protein, 146 aa, synthetic construct
MPPIRNVTVAYKFHMGLTGETNRLDSESGSISPKLRSIARYYDNPEMVPPDKCRCAVGSILSEGEESPSPELIDLYQKFGFKVFSFPAPSHVVTATFPYTTDLSKALATRRVHPALDTYIKERKLCAYPRLEIYQQDQIHFMCPLA

SEQ ID NO: 38 - protein, 146 aa, synthetic construct
MPPIRNVTVAYKFHMGLAGETGRLNRESSSISPKLRSIAKYYDNPRMVPPDKCRCAVGSILSEGEESPSPELIDLYQKFGFKVFSFPAPSHVVTATFPYTTQLSHTLATRRVHPALDTYIKERKLCAYPRLEIYQNDQIHFMCPLA

SEQ ID NO: 39 - protein, 146 aa, synthetic construct
MPPIRNVTVAYKFHMGLDGETRRLYMESRSISPKLRSIARYYDNPRMVPPDKCRCAVGSILSEGEESPSPELIDLYQKFGFKVFSFPAPSHVVTATFPYTTRLSDYLATRRVHPALDTYIKERKLCAYPRLEIYQDDQIHFMCPLA

SEQ ID NO: 40 - protein, 210 aa, synthetic construct
MGSTEKKIDFKKEEKKFYAPKRKPERIFVPEMNFLMVDGKGDRYGEELTKAWQSLLAIAGTIKMSKMGETRLDGYSDFVVPPAEGFSWSEGKFDNKDRDALLWTSILRQPDFVTEEVLEWAKEVARKKKPDVDTSRVKLVRFEEGECVQMMHVGLFSECVHTVAEMHQFMETEGLRNGTGAIRKHHYIWLSFFEKANPEKMKTILRLPVS

SEQ ID NO: 41 - protein, 210 aa, synthetic construct
MGSTEKKIDFKKEEKKFYAPKRKPERIFVPEMNFLMVDGKGDRYGEELTKAWQSLLAIAGTIKMSKMGETRLDGYSDFVVPPAEGFSWSEGKFDNKDRDALLWTSILRQPDFVTEEVLEWAKEVARKKKPDVDTSRVKLVRFEEGECVQMMHVGLFSEEVHTVAEMHQFMETEGLRNGTGAIRKHHYIELSFFEKANPEKMKTILRLPVS

SEQ ID NO: 42 - protein, 160 aa, synthetic construct
MDFECQFVCELKELAPVPALLIRTQTTMSEDRSLPTAGHFDILQLLAGQGKSPSGPPFARYFGMSAGTFEVEFGDPVEGGVEGSGRVVTGLTPSGKAASSLYIGPGGEIEAVYDALMKWVDDNGFDLSGEAYLIYLDNPAETAPDQLRTRVSLMLHESLE

SEQ ID NO: 43 - protein, 146 aa, synthetic construct; identical to SEQ ID NO: 24

SEQ ID NO: 44 - protein, 159 aa, synthetic construct
MEYQLQQLASLTLVGIKETYENGRQAAQHKKGFRQRCLQEGVIADLQLKNNGDLAGILGLCIPELDGKMSYMIAVTGDNSADIAKYDVITLASSKYMVFEAQGACMKAKTQKIEEVHHYIHQYQANTVKSAPFFTLPQDGDETSEKYITEIWMPVKGLE

SEQ ID NO: 45 - protein, 159 aa, synthetic construct
MEYQLQQLASLTLVGIKETYEHGRQASQHSSGFAQRCVQEGVIADLQLKNNGDLAGILGLCIPELDGKMSYMIAVTGDNSADIAKYDVITLASSKYMVFEAQGAGWKASRQKSEEVHHYIHQYQANTVKSAPFFTLVQDGDGTSEKYITEIWMPVKGLE

SEQ ID NO: 46 - protein, 159 aa, synthetic construct
MEYQLQQLASLTLVGIKETYENGRQALQHMSGFCQRCHQEGVIADLQLKNNGDLAGILGLCIPELDGKMSYMIAVTGDNSADIAKYDVITLASSKYMVFEAQGADFKAYPQKSEEVHHYIHQYQANTVKSAPFFHLRQDGDQTSEKYITEIWMPVKGLE

SEQ ID NO: 47 - protein, 146 aa, synthetic construct; identical to SEQ ID NO: 27

SEQ ID NO: 48 - protein, 146 aa, synthetic construct; identical to SEQ ID NO: 28

SEQ ID NO: 49 - protein, 146 aa, synthetic construct
MPPIRNVTVAYKFHMGLTRETDRLDRESHSISPKLRSIASYYDNPKMVPPDKCRCAVGSILSEGEESPSPELIDLYQKFGFKVFSFPAPSHVVTATFPYTTQLSKALATRRVHPALDTYIKERKLCAYPRLEIYQNDQIHFMCPLA

SEQ ID NO: 50 - protein, 146 aa, synthetic construct
MPPIRNVTVAYKFHMGLSEETGRLALESRSISPKLRSIARYYDNPNMVPPDKCRCAVGSILSEGEESPSPELIDLYQKFGFKVFSFPAPSHVVTATFPYTTRLSRTLATRRVHPALDTYIKERKLCAYPRLEIYQRDQIHFMCPLA

SEQ ID NO: 51 - protein, 146 aa, synthetic construct
MPPIRNVTVAYKFHMGLANETERLYTESRSISPKLRSIARYYDNPHMVPPDKCRCAVGSILSEGEESPSPELIDLYQKFGFKVFSFPAPSHVVTATFPYTTRLSHALATRRVHPALDTYIKERKLCAYPRLEIYQKDQIHFMCPLA

SEQ ID NO: 52 - protein, 146 aa, synthetic construct
MPPIRNVTVAYKFHMGLYQETHRLALESNSISPKLRSIANYYDNPQMVPPDKCRCAVGSILSEGEESPSPELIDLYQKFGFKVFSFPAPSHVVTATFPYTTNLSHALATRRVHPALDTYIKERKLCAYPRLEIYQGDQIHFMCPLA

SEQ ID NO: 53 - protein, 146 aa, synthetic construct; identical to SEQ ID NO: 39

SEQ ID NO: 54 - protein, 146 aa, synthetic construct
MPPIRNVTVAYKFHMGLSGETGRLALESRSISPKLRSIAKYYDNPGMVPPDKCRCAVGSILSEGEESPSPELIDLYQKFGFKVFSFPAPSHVVTATFPYTTQLSGSLATRRVHPALDTYIKERKLCAYPRLEIYQHDQIHFMCPLA

SEQ ID NO: 55 - protein, 71 aa, synthetic construct
MPPIRNVTVAYKFHMGLAGETGRLALESNSISPKCAASPNITTTRVWFLRISAVVQLVASSPKARSPHLRS

SEQ ID NO: 56 - protein, 51 aa, synthetic construct
MPPIRNVTVAYKFHMGLSGETRPFVFGKRQHQPETAQHRQILRQPEHGSSG

SEQ ID NO: 57 - protein, 146 aa, synthetic construct
MPPIRNVTVAYKFHMGLYGETNRLSLESKSISPKLRSIARYYDNPRMVPPDKCRCAVGSILSEGEESPSPELIDLYQKFGFKVFSFPAPSHVVTATFPYTTSLSRNLATRRVHPALDTYIKERKLCAYPRLEIYQSDQIHFMCPLA

SEQ ID NO: 58 - protein, 149 aa, synthetic construct
MPPIRNVTVAYKFHMGLYGETGRLSMESRSISPKLRSIANYYDNPGMVPPDKCRCAVGSILSEGEESPSPELIDLYQKFGFKVFSFPAPSHVVTATFPYTTRLSRYLATRRVHPALDTYIKERKLCAYPRLEIYQHDQIHFMCPLAGSA

SEQ ID NO: 59 - protein, 149 aa, synthetic construct
MPPIRNVTVAYKFHMGLSGETERLARESESISPKLRSIAGYYDNPEMVPPDKCRCAVGSILSEGEESPSPELIDLYQKFGFKVFSFPAPSHVVTATFPYTTGLSSSLATRRVHPALDTYIKERKLCAYPRLEIYQSDQIHFMCPLAGSA

SEQ ID NO: 60 - protein, 146 aa, synthetic construct
MPPIKNVTVAYKFHMGLARETSCLYWESRSISPKLRSIARYYDNPRMVPPDKCRCAVGSILSEGEESPSPELIDLYQKFGFKVFSLPAPSHVVTATFPYTTGLSGALATRRVHPALDTYIKERKLCGYPRLEIYQRDQIHFMCPLA

SEQ ID NO: 61 - protein, 146 aa, synthetic construct
MPPIKNVTVAYKFHMGLARETSRLYWESRSISPKLRSIARYYDNPRMVPPDKCRCAVGSILSEGEESPSPELIDLYQKFGFKVFSLPAPSHVVTATFPYTTGLSGALATRRVHPALDTYIKERKLCAYPRLEIYQRDQIHFMCPLA

SEQ ID NO: 62 - protein, 146 aa, synthetic construct
MPPIRNVTVAYKFHMGLDNETDRLSMESRSISPKLRSIAGYYDNPNMVPPDKCRCAVGSILSEGEESPSPELIDLYQKFGFKVFSFPAPSHVVTATFPYTTSLSKSLATRRVHPALDTYIKERKLCAYPRLEIYQHDQIHFMCPLA

SEQ ID NO: 63 - protein, 150 aa, synthetic construct
HKDVTVAYKFHMGLYRETRRLSWESNSISPKLRSIARYYDNPDMVPPDKCRCAVGSILSEGEESPSPELIDLYQKFGFKVFSFPAPSHVVTATFPYTTGLSQTLATRRVHPALDTYIKERKLCAYPRLEIYQRDQIHFMCPLAKLAAALE

SEQ ID NO: 64 - protein, 149 aa, synthetic construct
KDVTVAYKFHMGLARETRRLTSESHSISPKLRSIAKYYDNPSMVPPDKCRCAVGSILSEGEESPSPELIDLYQKFGFKVFSFPAPSHVVTATFPYTTELSRDLATRRVHPALDTYIKERKLCAYPRLEIYQRDQIHFMCPLAKLAAALE

SEQ ID NO: 65 - protein, 202 aa, synthetic construct
MGTFFYGGSRGKRNNFKTEEYGGGGSMKELPKRYFAYREVKNQYSEIKEALELAEANFPGKTPISFPIDKGCSYNTNTCKRGAGFFLTDDGTAPDEATIQKAKDLGFKTGSFPKDPNGYEITIPEGGEKAVRKFYTELLPKAIEEVQKYAKNPIIVETYANGKFVGAILYNDSAWSHPQFEKGGGSGGGSGGSAWSHPQFEK

SEQ ID NO: 66 - protein, 202 aa, synthetic construct
MGTFFYGGSRGKRNNFKTEEYGGGGSTKPRKGFYGAYKEVNGDPEKVKEAIDTARKIDPNGIEMVIVMEDYDSTNLEERKVKAGKIINPTGKEPSEEIIKKYKDNGFEPFKTPELKKVFYYEAEAESEEEAEKALCEGLPAFVESIKKAGESPYPLYLWSNGKKLGAWVPIESAWSHPQFEKGGGSGGGSGGSAWSHPQFEK

SEQ ID NO: 67 - protein, 146 aa, synthetic construct
EPPLPKRYYAYKEVLNIPTEVQKALKEAEANFKGKTLISFPIDKGCSPNNNLCKLGAGFYLNETGEEPSEEDVNKAKELGFKTGAFEKSPKGKELKLPEGGEKAIRHFFEKLLPEAIEEVQKYFPAPQIVYLYENGKLIGFELYPE

SEQ ID NO: 68 - protein, 146 aa, synthetic construct
AEKIPKVYYAAKIVSNRYEEVKAALEEAEKLKPGATQLEFFIDKGSSRSDKSAKVGAGFILASDGEPPPEELIEKARENGFEVGSFPASEKGSSIGVPGGDEENIKKLYTVLEPKVMEDVRKYGAEPFIVYLYRNGKWSGVAAFAE

SEQ ID NO: 69 - protein, 146 aa, synthetic construct
GPSLPAHSGFAKETTSDPEEVSAAIRTARAIDPEGTLFLTFTSDPDEVSVEEARSFAGVITRWDGSEIDPALAERAAAEGFTPFTEPARPLATAGEAEAATPEEALEGVARRLPEAVAELLAAGRRPYPLTLWRRGNRAGWWVPAE

SEQ ID NO: 70 - protein, 146 aa, synthetic construct
GPSLPAHSGFARETTADPEEVGAAIRTARALDPEGTLFLTFTADPEEVSVEEARSRAGVITRWDGSEVDPELAERARAEGFTPFTEPARPLATAGSAEAATPEEALEGVARVLPEAIAALRAAGREPYPLTLWQRGNRAGWWVPAE

SEQ ID NO: 71 - protein, 146 aa, synthetic construct
CYSGSPPFLLYTTDSLNKAEAEAAIEKGRSLSPEGKVYLTTRSTTGEKPKENSYMAVGIQPDRSGKKPNEELTKKIKEAGFKPADSPPCPILACGEKSASSWKEALEKVYEELPKVVKELLEKGKKPNYLWEWVSGNSAGYWVPEA

SEQ ID NO: 72 - protein, 146 aa, synthetic construct
AFTGPPPWRLYAKEGLTPAEMAAAIAQGLSLDPSGRVGFYNDDTSGAPWDPSRKGGVFLVLNPDGVTVDTALEAAAKAAGFKDQGTLPWPILACATAEGSNYQEALAKLYETSPKIAAELRAAGLTPGPIYYFVWGNKAGLLIPAA

SEQ ID NO: 73 - protein, 146 aa, synthetic construct
GCRTGDFWYAYRFGLGDPAKIWEIWEEAEKLDPEPHPAGWVAGPLSKVAEGPVTYAAGQILSWGPEPPPEELIKKYTEAGFAIGYRPDFPEYISTSAPIPSKEAALETLKKLSEEIYKQLEAEGKTDRVIYWWRDGDKLCAWAPLD

SEQ ID NO: 74 - protein, 146 aa, synthetic construct
CSRWGSFWYAYRRGGGDLSYLPEDIEKALAIDPNYTPCGWVAGPEDLFPRGELPYAAGRILSWGSEPPAVEVTERFAKEGFAVGYVEESPAAILGEATISNKESFLSALKKLVTELWEAQKALGYDERPIRFWEDGDKLCFAAPLP

SEQ ID NO: 75 - protein, 146 aa, synthetic construct
GSRTGAFWYAYRLGNGDISTIWPTIEEARKLSPEYTPAGFVYGPLSKYPDGNILFGAGRILSWGEEPPPQELIEKFTQAGYAIGFVPDFPAYISTSSTISNKESFLSTLTKLSQEIYDELEKLGKSDTPVYFWIDGDHLCFYAPLD

SEQ ID NO: 76 - protein, 146 aa, synthetic construct
CSFSGAFWYAYRLGNGDLSTVWDTIKEAEKLSKEYTPSGYLYGHPEDYPDGNQLFGAGRILSWGEEPPPKELIEKFTKAGYAVGFVPDFPASIENSSTISNEKSFLDSLTKLWQEIQDKQKALGKEDHPVYFWIDGDHICFYAPLD

SEQ ID NO: 77 - protein, 146 aa, synthetic construct
MTRRGAFWYAHRLGNGDLSTIWPTIEEARKLSPEYTPSGYVFGPPEKYPDGNQLFGAGRILSWGTEPPPQELIEKFTQAGYAVGFVEDVPAFIENSGKVSDKKSFLDTLKKLVAELYKKAKALGKEDHPVYFWLDGDEIAFRMDLG

SEQ ID NO: 78 - protein, 146 aa, synthetic construct
CTEFGSYWYAYKRGSGSLELLPKEIEKAKSIDPTYTPAGSVWGPEEEFEPDSLPYTAGQILSWGKEPPGPEITEKFIEHGFSVGYRTAPKKYLTGEAKISDEESFLKALKELVKELYDKQVALGERNYPIEFWLDGDTLCFRAPLA

SEQ ID NO: 79 - protein, 146 aa, synthetic construct
CSSFGSYWYAYKRGAGDYSELPKLIEEAKSIDPTYTPAGWVWGPESEFEPNKVPFRAGVILSWGRTPPGPEITEKFVEHGFSVGYRTAPSRYLTGSAKITNEESFLKALKEKVQELHDKQTALGEKNYPIYFWLDGDTLCFVAPLA

SEQ ID NO: 80 - protein, 146 aa, synthetic construct
CSDFGDFWYAYRRGAGDLAELPKIIEEAKAIDPTYTPAGWVWAPEEEFEPNKLPFSAGVILSWGKEPPGPEVTEKFIEHGFSVGYRVAPKKYLTGSAPVSNEESFLEALKKGVQELYDKQKALGESNYPYWFWLDGDTLCFKAPLA

SEQ ID NO: 81 - protein, 146 aa, synthetic construct
CSSFGSYWYAYKRGAGSLAELPEEIKKALSIDPTYTPAGWVWGPESLFEPNKIPYRAGVILSWGKEPPGPEITEKFIEHGFSVGYRTAPKKYLTGSAKISDDESFLAALKKLVQDLYDKQVASGNLNTPIEFWLDGDTLCFIAPLA

SEQ ID NO: 82 - protein, 146 aa, synthetic construct
GCRTGDAWVAYRYNLGDPEKFWEIWKEAEKLDPTGHPAGWVYRPLSDVEEGPVLGAAGQILSWGPEPPPEELIKKYVENGFVISYIPDFPDRLEAYMPVPNEEDALEALKELSEKIYEELKKLGLEDRPIYWWRDGDKLCAWAPLD

SEQ ID NO: 83 - protein, 146 aa, synthetic construct
MVKPKGFWVAYRETSGTMEDLYAELKKAKNDFGNAQPVIIIYNDPLEVTPEEFKGAAGAILSSGPRAPPEEVLQAAKNKGYNVDYIPATDKAWYKPLPLKGEEGYKYFFEKEYPKLKEELKAKGLPTTPLIIFDGENGYTVTVPQD

SEQ ID NO: 84 - protein, 146 aa, synthetic construct
GRTLHPFWYAYKPWEGSLDDLAKGLEEAEKAAKNALKFAWLLNDPLEVSEKELKGGAGFILSATSRKPAPEVITKAKEQGWEVGSFPGSKNAWFLKLPMKGAEGLKEFFNNLWPKIHEELTSKGLPTIPAYFFFGKDGTTFAVPND

SEQ ID NO: 85 - protein, 146 aa, synthetic construct
DKPPKGMWVAVKEGSGPLEKAFELLEEAKELFGGATPIIRFYNDPLEVSEEEFRYEAGVILSTGDKPPPEEIINRAKELGYKVEYVPKATNAINFSVPYKGKEGFEKFFNEIWPKKREEFKKKGLKTTPLTLIETGDSWDVYIPQD

SEQ ID NO: 86 - protein, 146 aa, synthetic construct
GRKLHPFWVAYKKIEGTIEELAKGIEEAEKAFKNAMKIAGLYNDPLEVSEDELKGFAGAILSATSKKPAPEVIEKAKELGWDVGSIPGSKNAVFKKLPMKGAEGLKYFFENLYPEWHKELTSKGLPTIPLYFFFSKDGTTVAVPND

SEQ ID NO: 87 - protein, 146 aa, synthetic construct
MKAPLGFWVAYKEGSGKLEDAYKALKEAEKAFGKATPIIMFWNDPLEVTDEEFKYAAGVILSCGPKPPQQEIIDLAKKKGFKVDYIPAAKEAYSKPIPKKGEEGFNYFFNEEYPKLREELKSKGKKTTPLYIIEGTTGLDVYVPQD

SEQ ID NO: 88 - protein, 146 aa, synthetic construct
MKFKGGDPYAWRDTSGDKEEIKKIWEEGLALAKEYGITDAQILATLKADEDYKVTSAGVVVRRKPGQTWSDIVKVWEAAGFKAGTLAPFSGAIELPTPAKTLDEKVNVLKEEVPKLIKEFEEKGCSPEPAVFVFEGDKPVLLTNAC

SEQ ID NO: 89 - protein, 146 aa, synthetic construct
GPPSGAFYIAYKPIDGSIEKFIDIWDDASKLAPGRTLWAAIFTSFSEAPIEKVKGIAGVILDSGTGKASPEIAALYEKAGWKTKSVPKAEGAVAYPVITPTREEQLKFFKEELPKIEAELKAKGIKPNPLIIVYGGNTPAVLYSKS

SEQ ID NO: 90 - protein, 146 aa, synthetic construct
ALPTGGEFVAYREYSGDPEEIKAIWEEAKALAKEYGVLNAVFAGWYKTDENNEIRFAGVIVGTPDGSGEEERKAFFEAAGFKAERIAPLQGFKKLPTKAPTLEEKVAVLKTEVPKVLEEYKEKGVDPNPAILVFLEDTPILWYDSG

SEQ ID NO: 91 - protein, 146 aa, synthetic construct
GLPKGGFYIAYKPISGPLSEILDIWNDCSTLASGVNKLFAIFTAYEEKSLDNVSGYAGCILDGQNGDEQPELAKYFEDKGFKTAQVEKPIGAIQAPKPYPTREEKLKFLKEELPKIIAELKAKGIVPNPLMVVFGGPQPTVLYKPK

SEQ ID NO: 92 - protein, 146 aa, synthetic construct
SLPTGGEFVAYKEISGDPEEIEEVWKEAEALAKEYGVLNARFAGWYKTDENNEIRWAGVIVGRKDGSGEEERKAFFEAAGFKAGRIAPLQGFKKLPTPAPTLKEKVDVLKKEVPKVLEEAKKKGIDPNPAKLVFLEDTPILLWDSE

SEQ ID NO: 93 - protein, 146 aa, synthetic construct
CGCAGKRKVVYKELEGHLNLALDAIEEALQIDPNGTPIIIIYDDPETTPLDKIKFKAGVIIACGDEEPSEELIQKFKEAGFEYEEIPKPKAAIGAWAPYSSPEDFENFLDNTIPALKTPLDAKGYPHYPLYITIGSNNICALVRDY

SEQ ID NO: 94 - protein, 146 aa, synthetic construct
CGCAGKRKVVYKELEGHLNLALDAIEEALEIDPNGVPIIIIYDDPETTPLDKIKFKAGVVIACGDEEPSEELIEKFREAGFKFEELPKPKAAVSASAPYSSPEDFEKFLDETIPKLKEPLDAKGYKHYPLYISIGSNKICAYVPDY

SEQ ID NO: 95 - protein, 146 aa, synthetic construct
CGCNGKRKVVYKELKGHLNLALDAIEEALKIDPNGTPIIIIYDDPETTPLDEIEFKAGVVIACDDEEPDPELIEKFKKAGFKYETIPKPKAAISAYAPYSSPEDFENFLDNTIPNLKKPLDAKGYPHYPLEISIGSNNICAAIRDY

SEQ ID NO: 96 - protein, 146 aa, synthetic construct
MGPSGKLYVAYKEHKGPFNLALDTIEEALKIDPSGTPIIILYNDPETTPLDEIEFKAGIVYASGDVEPDESLINKFKEAGFEYEKLPKAKSSVGVTMPLKDPEDVYKFLDEAVPKLKEPLKAVGLPNYPLLLQFGNNRISASVRDY

SEQ ID NO: 97 - protein, 146 aa, synthetic construct
CGCNGKRKVIYKKLKGHLNLALDAIEEALKIDPKGTPIIIIYDDPETTDLNKIEFKAGVVIACEDEEPDPELIEKFKEAGFEFEELPKPKAAICAYAPYSSPEDFENFLDNTIPKFKKPADEKGYPHYPLYISIGSNNICFCLPDY

SEQ ID NO: 98 - protein, 146 aa, synthetic construct
MPLENGFRFAYKEFSGDLSLIDEGVEAAKKAFPDKPPFVVLDSIEEIDGRYNVSGQVGVVLAPNGEEMTKEDYERPKKLGWKVSSRGKPLWTWSFPREPGESVWETLWRHLPPERLKELLESTGKPLSDLVLVFSDEEVTLVLTSS

SEQ ID NO: 99 - protein, 146 aa, synthetic construct
MELNNGFTYAYKPFSGEISIIDKKLEEARTKFPDYPPFLQIESFGEENGKKVISGYAGVILSPNGKKPTLEDWIKPKENGYEVSRTGKPKGTVTFPRKPGEKVSDLIFKLFPPEEIKKILEETGIKLEDLFIVFEKDTVTLVIGDS

SEQ ID NO: 100 - protein, 146 aa, synthetic construct
MDLNSPFTYAYKPFEGDIKLIDDKIAEAKKIFPDKPPFIVIESYGYKDGKLTIKGYAGVVLSPNGKRPNKEDIEKPKEHGYETSRSNSPKGAITFPRKPGTPLSELIFELFPPEELEAWLKETGISLKDLLIVLTPDKVTVVILDS

SEQ ID NO: 101 - protein, 146 aa, synthetic construct
MDLSKGFYFAYKPFSGDESILDEKVNEAKALFPDKPPFVVIESYKEENGKRLISGYAGVVLAPNGKEPTKEDYEKPKANGYDVSWTGDPKDTWSFPIEKGEKISDWFWKHFPLEKLEEIKKELGVKLSDLMFVFSDSEATLVIRSE

SEQ ID NO: 102 - protein, 146 aa, synthetic construct
MPLENGFWFAYKPFSGPLPLVDEGVKEAKEAFPDKPPFVVFESLEEIDGEWNVSGYVGVVLAPNGKEPSKEDYERPKKLGWKVSRRGKPLWTWSAPIKPGETVTEWLFRHLPPERLAEVLESTGLPLSKLVFVFSEEEATAVLTTS

SEQ ID NO: 103 - protein, 146 aa, synthetic construct
MDLSSSFYYAYKPFSGDVSLLDTKIAEAKAIFPDLPPFVIIDSYGYENGKYTISGYAGVVLAPNGKRPTKELIEKPKEHGYETSRTNSPKGQVTFPREPGTPVSELIWQLFPPEELKKYLEETGISLKDLLIVFTPDEVTVVILSS

SEQ ID NO: 104 - protein, 149 aa, synthetic construct; identical to SEQ ID NO: 58

SEQ ID NO: 105 - protein, 149 aa, synthetic construct; identical to SEQ ID NO: 59

SEQ ID NO: 106 - protein, 146 aa, synthetic construct
SSSSATYYACTKKFSGSIDVLKELIPTAKKIDPNLIFGFNIDMPETGYTVGNVTSTSKVEFFYGIEEPSEELINKCKEAGFTPEKKTGVKAKLGFCISYKNEEEIIEAIKEGTTKMYNEAKAKGLKPTPVRGYQCGDKLAFYLPEE

SEQ ID NO: 107 - protein, 146 aa, synthetic construct
SNPSSSYNACCKKFSGDISVLKEFIPLIKKIDPNLIFGFSISMPESGYTPGKVTSESKVCFFTGVEEPTEEEIEKCKELGFEYEKYEGTLAKLGFSIKYSNDEEIIDAIKKGVTEMYEKAKAAGLKPTPVRGYKCGDSLCFYLPEK

SEQ ID NO: 108 - protein, 146 aa, synthetic construct
AADTGTYWVFSKKFSGDLEKILELRKKAKEIDPNLVFGFNIEVPEDSYEPGKITEGSSLEFYYGSLEPSEELIKKFKEAGFTPVKLEGKPAALGYSIPYTNEEDILEAIKKGVSGMYDKAKELGLKPSPVYGYEDGDTLCFWLPAA

SEQ ID NO: 109 - protein, 146 aa, synthetic construct
ADPTATYYAITKNFSGDLSVILELIPKAKEIDPNLIFGFNISMPETGYTPGNVTSTSKVEFYYGIESPSEELIKKAKEAGFTPVQSTGVEAKLGFEIPFKNEEDRVEVLKTGLTKMYNEAKAKGLKPTPVRGYECGDKLAFYLPKS

SEQ ID NO: 110 - protein, 146 aa, synthetic construct
GPPSSNYPACYKPFSGDLSKFKEYLPEIKKLDPNAVFGFVVDLPDSGWEKGKVTEGSYVTFYTGLETPTPEFVEKCAKLGMKTSMAPGSLCKYGGSISFSNDEEIIEKIAEGVWSMYEKAKALGLKPTPVAGYIDGDKICICLPEN

SEQ ID NO: 111 - protein, 122 aa, synthetic construct; identical to SEQ ID NO: 18

SEQ ID NO: 112 - protein, 122 aa, synthetic construct; identical to SEQ ID NO: 19

SEQ ID NO: 113 - protein, 122 aa, synthetic construct
MQVQLVESGGGSVQAGGSLRLSCTASGARHIKYSAFYLGWFRQAPGQEREAVAAIASEGGKTYYADSVKGRFTISRDNAKNTVTLQMNNLKPVDTAIYYCAANDYDYKTFRYWGQGTQVTVS

SEQ ID NO: 114 - protein, 124 aa, synthetic construct
MQVQLVESGGGSVQAGGSLRLSCTASGSRQFQYDRFSLGWFRQAPGQEREAVAAIDTGGGNTYYADSVKGRFTISRDNAKNTVTLQMNNLKPVDTAIYYCAALRSGYNNLSSWGQGTQVTVSKL

SEQ ID NO: 115 - protein, 124 aa, synthetic construct
MQVQLVESGGGSVQAGGSLRLSCTASGAERIRYDIFDLGWFRQAPGQEREAVAAIDTRGGITYYADSVKGRFTISRDNAKNTVTLQMNNLKPVDTAIYYCAAQRNRNRYFGSWGQGTQVTVSKL

SEQ ID NO: 116 - protein, 124 aa, synthetic construct
MQVQLVESGGGSVQAGGSLRLSCTASGNEGHRYNIFALGWFRQAPGQEREAVAAIDSGGGRTYYADSVKGRFTISRDNAKNTVTLQMNNLKPVDTAIYYCAALHTHSITFKYWGQGTQVTVSKL

SEQ ID NO: 117 - protein, 125 aa, synthetic construct
MQVQLVESGGGSVQAGGSLRLSCTASGSERYNYSRFALGWFRQAPGQEREAVAAIYLNGGKTYYADSVKGRFTISRDNAKNTVTLQMNNLKPVDTAIYYCAAASNSTVTLRSWGQGTQVTVSGSA

SEQ ID NO: 118 - protein, 125 aa, synthetic construct
MQVQLVESGGGSVQAGGSLRLSCTASGDRRLRYYTFDLGWFRQAPGQEREAVAAIDWGGGITYYADSVKGRFTISRDNAKNTVTLQMNNLKPVDTAIYYCAAADTEYVSFGYWGQGTQVTVSGSA

SEQ ID NO: 119 - protein, 123 aa, synthetic construct
MQVQLVESGGGSVQAGGSLRLSCTASGSHGIDYSGFDLGWFRQAPGQEREAVAAIYLDGGSTYYADSVKGRFTISRDNAKNTVTLQMNNLKPVDTAIYYAAQNYNYVSYDYWGQGTQVTVSKL

SEQ ID NO: 120 - protein, 125 aa, synthetic construct; identical to SEQ ID NO: 117

SEQ ID NO: 121 - protein, 125 aa, synthetic construct; identical to SEQ ID NO: 118

SEQ ID NO: 122 - protein, 187 aa, synthetic construct; identical to SEQ ID NO: 8

SEQ ID NO: 123 - protein, 187 aa, synthetic construct
MGSSVEVHPVLEKLKAINNYNPKDFDWNLKNGRVFIIKSYSEDDIHRSIKYSIWCSTEHGNKRLDAAYRSLNGKGPLYLLFSVNGSGHFCGVAEMKSVVDYNAYAGVWSQDKWKGKFEVKWIFVKDVPNNQLRHIRLENNDNKPVTNSRDTQEVPLEKAKQVLKIIATFKHTTSIFDDFAHYEKRQE

SEQ ID NO: 124 - protein, 122 aa, synthetic construct; identical to SEQ ID NO: 113

SEQ ID NO: 125 - protein, 188 aa, synthetic construct
MGSSVEVHPVLEKLKAINNYNPKDFDWNLKNGRVFIIKSSSEDDIHRSIKYSIMTSSEHGNKRLDAAYRSLNGKGPLYLLFSVVGSGHFCGVAEMKSVVDYNAYAGVDSQDKHKGKFEVKWIFVKDVPKNQLRHIRLENNDNKPVTGSWRTQEVPLEKAKQVLKIIATFKHTTSILDDFAHYEKRQES

SEQ ID NO: 126 - protein, 187 aa, synthetic construct
MGSSVEVHPVLEKLKAINNYNPKDFDWNLKNGRVFIIKSLSEDDIHRSIKYSIAQSAEHGNKRLDAAYRSLNGKGPLYLLFSVPGSGHFCGVAEMKSVVDYNAYAGVWSQDKGKGKFEVKWIFVKDVPCNQLRHIRLENNDNKPVLSQPTQEVPLEKAKQVLKIIATFKHTTSIFDDFAHYEKRQES

SEQ ID NO: 127 - protein, 188 aa, synthetic construct
MGSSVEVHPVLEKLKAINNYNPKDFDWNLKNGRVFIIKSASEDDIHRSIKYSICESGEHGNKRLDAAYRSLNGKSPLYLLFSVAGSGHFCGVAEMKSVVDYNAYAGVPSQDKAKGKFEVKWIFVKDVPKNQLRHIRLKNNDNKPVTCSCRTQEVPLEKAKQVLKIIATFKHTTSIFDDFAHYEKRQES

SEQ ID NO: 128 - protein, 188 aa, synthetic construct
MGSSVEVHPVLEKLKAINNYNPKDFDWNLKNSRVFIIKSTSEDDIHRSIKYSIRRSMEHGNKRLDAAYHSLNGKGPLYLLFSVTGSGHFCGVAEMKSVVDYNAYAGVGSQDKTKGKFEVKWIFVKDVPKNQLRHIRLENNDNKPVTDSLPTQEVPLEKAKQVLKIIATFKHTTSIFDDFAHYEKRQES

SEQ ID NO: 129 - protein, 188 aa, synthetic construct
MGSSVEVHPVLEKLKAINNYNPKDFDWNLKNGRVFIIKSHSEDDIHRSIKYSIRQSLEHGNKRLDAAYRSLNGKGPLYLLFSVVGSGHFCGVAEMKSVVDYNAYAGLRSQDKTKGKFEVKWIFVKDVPRNQLRHIRLENNDNKPVTTSKWTQEVPLEKAKQVLKIIATFKHTTSIFDDFAHYEKRQES

SEQ ID NO: 130 - protein, 188 aa, synthetic construct
MGSSVEVHPVLEKLKAINNYNPKDFDWNLKNGRVFIIKSMSEDDIHRSIKYSIVSSEEHGNKRLDAAYRSLNGKGPLYLLFSVAGSGHFCGVAEMKSVVDYNAYAGVDSQDKAKGKFEVKCIFVKDVPLNQLRHIRLENNDNKPVTCSDRTQEVPLEKAKQVLKIIATFKHTTSIFDDFAHYEKRQES

SEQ ID NO: 131 - protein, 188 aa, synthetic construct; identical to SEQ ID NO: 129

SEQ ID NO: 132 - protein, 188 aa, synthetic construct
MGSSVEVHPVLEKLKAINNYNPKDFDWNLKNGRVFIIKSHSEDDIHRSIKYSISGSSEHGNKRLDAAYRSLNGKGPLYLLFSVRGSGHFCGVAEMKSVVDYNAYAGLRSQDKTKGKFEVKWIFVKDVPRNQLRHIRLENNDNKPVTTSKWTQEVPLEKAKQVLKIIATFKHTTSIFDDFAHYEKRQES

SEQ ID NO: 133 - protein, 188 aa, synthetic construct
MGSSVEVHPVLEKLKAINNYNPKDFDWNLKNGRVFIIKSVSEDDIHRSIKYSILPSGEHGNKRLDAAYRSLNGKGPLYLLFSVEGSGHFCVVAEMKSVVDYNAYAGLRSQDKPKGKFEVKWIFVKDVPRNQLRHIRLENNDNKPVTNSAVTQEVPLEKAKQVLKIIATFKHTTSIFDDFAHYEKRQES

SEQ ID NO: 134 - protein, 188 aa, synthetic construct
MGSSVEVHPVLEKLKAINNYNPKDFDWNLKNGRVFIIKSASEDDIHRSIKYSIWTSGEHGNKRLDAAYRSLNGKGPLYLLFSVAGSGHFCGVAEMKSVVDYNAYAGLRSQDKTKGKFEVKWIFVKDVPRNQLRHIRLENNDNKPVTTSACTQEVPLEKAKQVLKIIATFKHTTSIFDDFAHYEKRQES

SEQ ID NO: 135 - protein, 188 aa, synthetic construct
MGSSVEVHPVLEKLKAINNYNPKDFDWNLKNGRVFIIKSRSEDDIHRSIKYSIMPSWEHGNKRLDAAYRSLNGKGPLYLLFSVPGSGHFCGVAEMRSVVDYNAYAGVMSQDKSKGKFEVKWIFVKDVPRNQLRHIRLENNDNKPVTRSPPTQEVPLEKAKQVLKIIATFKHTTSIFDDFAHYEKRQES

SEQ ID NO: 136 - protein, 189 aa, synthetic construct
MGSSVEVHPVLEKLKAINNYNPKDFDWNLKNGRVFIIKSSSEDDIHRSIKYSIRSSDEHGNKRLDAAYRSLNGKGPLYLLFSVTGSGHFCGVAEMKSVVDYNAYAGVPSQDKHKGKFEVKWIFVKDVSVNQLRHIRLENNDNKPVTDSDPTQEVPLEKAKQLLKIIATFKHTTSIFDDFAHYEKRQESA

SEQ ID NO: 137 - protein, 189 aa, synthetic construct
MGSSVEVHPVLEKLKAINNYNPKDFDWNLKNGRVFIIKSWSEDDIHRSIKYSIRASFEHGNKRLDAAYRSLNGKGPLYLLFSVCGSGHFCGVAEMKSVVDYNAYAGVPSQDKWKGKFEVKWIFVKDVPQNQLRHIRLENNDNKPVTSSTTTQEVPLEKAKQVLKIIATFKHTTSIFDDFAHYEKRQESA

SEQ ID NO: 138 - protein, 189 aa, synthetic construct
MGSSVEVHPVLEKLKAINNYNPKDFDWNLKNGRVFIIKSCSEDDIHRSIKYSIDTSLEHGNKRLDAAYRSLNGKGPLYLLFSVSGSGHFCGVAEMKSVVDYNAYAGVRSQDKGKGKFEVKWIFVKDVPHNQLRHIRLENNDNKPVTCSTNTQEVPLEKAKQVLKIIATFKHTTSIFDDFAHYEKRQESA

SEQ ID NO: 139 - protein, 237 aa, synthetic construct; identical to SEQ ID NO: 14

SEQ ID NO: 140 - protein, 237 aa, synthetic construct
MTNKELQAIRKLLMLDVSEAAEHIGRVSARSWQYWESGRSAVPDDVEQEMLDLASVRIEMMSAVDKCLVNGERPQLRFYNKLGEYLADNPEHNVIGWRLSQSVAALYYTEGHADLIGGGGSMTNKELQAIRKLLMLDVSEAAEHIGRVSARTWHYWESGRSAVPDDVEQEMLDLASVRIEMMSAIDKRLADGERPKLRFYNKLDEYLADNPDHNVIGWRLSQSVAALYYTEGHADLI

SEQ ID NO: 141 - protein, 237 aa, synthetic construct
MTNKELQAIRKLLMLDVSEAAEHIGRVTAQNWEYWESGESAVPDGVEQEMLDLASVRIEMMSAIDKRLADGERPKLRFYNKLDEYLADNPDHNVIGWRLSQSVAALYYTEGHADLIGGGGSMTNKELQAIRKLLMLDVSEAAEHIGRVDARAWRYWESGQSAVPDEVEQEMLDLASVRIEMMSAIDKRLADRERPKLRFYNKLDEYLADNPDHNVIGWRLSQSVAALYYTEGHADLI

SEQ ID NO: 142 - protein, 237 aa, synthetic construct; identical to SEQ ID NO: 141

SEQ ID NO: 143 - protein, 237 aa, synthetic construct
MTNKELQAIRKLLMLDVSEAAEHIGRVNAQYWRGWESGESAVPDQVEQEMLDLASVRIEMMSAIDKRLADGERPKLRFYNKLDEYLADNPDHNVIGWRLSQSVAALYYTEGHADLIGGGGSMTNKELQAIRKLLMLDVSEAAEHIGRVDAHTWQDWESGGSAVPDQVEQEMLDLASVRIEMMSAIDKRLADGERPKLRFYNKLDEYLTDNPDHNVIGWRLSQSVAALYYTEGHADLI

SEQ ID NO: 144 - protein, 237 aa, synthetic construct
MTNKELQAIRKLLILDVSEAAEHIGRVYAGNWRHWESGHSAVPDRVEQEMLDLASVRIEMMSEIDKRLADGERPKLRFYNKLDEYLADNPDHNVIGWRLSQSVAALYYTEGHADLIGGGGSMTNKELQAIRKLLMLDVSEAAEHIGRVDAQTWQDWESGGSAVPDRVEQEMLDLASVRIEMMSAIDKRLADGERPKLRFYNKLDEYLADNPDHNVIGWRLSQSVAALYYTEGHADLI

SEQ ID NO: 145 - protein, 237 aa, synthetic construct
MTNKELQAIRKLLMLDVSEAAEHIGRVTAEYWHDWESGGSAVPDGVEQEMLDLASVRIEMMSAIDKRLADGERPKLRFYNKLDEYLADNPDHNVIGWRLSQSVAALYYTEGHADLIGGGGSMTNKELQAIRKLLMLDVSEAAEHIGRVAAHDWHRWESGGSAVPDRVEQEMLDLASVRIEMMSAIDKRLADGERPKLRFYNKLDEYLADNPDHNVIGWRLSQSVAALYYTEGHADLI

SEQ ID NO: 146 - protein, 237 aa, synthetic construct
MTNKELQAIRKLLMLDVSEAAEHIGRVTARDWHRWESGESAVPDHVEQEMLDLASVRIEMMSAIDKRLADGERPKLRFYNKLDEYLADNPDHNVIGWRLSQSVAALYYTEGHADLIGGGGSMTNKELQAIRKLLMLDVSEAAEHIGRVNAEYWEDWESGQSAVPDGVEQEMLDLASVRIEMMSAIDKRLADGERPKLRFYNKLDEYLADNPDHNVIGWRLSQSVAALYYTEGHADLI

SEQ ID NO: 147 - protein, 237 aa, synthetic construct
MTNKELQAIRKLLMLGVSEATEHIGRVYAETWRGWESGQSAVPDGVEQEMLDLASVRIEMMSAIDKRLADGERPKLRFYNKLDEYLADNPDHNVIGWRLSQSVAALYYTEGHADLIGGGGSMTNKELQAIRKLLMLDVSEAAEHIGRVAARYWRDWESGQSAVPDGVEQEMLDLASVRIEMMSAIDKRLADGERPKLRFYNKLDEYLADNPDHNVIGWRLSQSVAALYYTEGHADLI

SEQ ID NO: 148 - protein, 237 aa, synthetic construct
MTNKELQAIRKLLMLDVSEAAEHIGRVSARNWHYWESGESAVPDRVEQEMLDLASVRIEMMSAIDKRLADGERPKLRFYNKLDEYLADNPDHNVIGWRLSQSVAALYYTEGHADLIGGGGSMTNKELQAIRKLLMLDVSEAAEHIGRVNAHDWQGWESGHSAVPDGVEQEMLDLASVRIEMMSAIDKRLADGERPKLRFYNKLDEYLADNPDHNVIGWRLSQSVAALYYTEGHADLI

SEQ ID NO: 149 - DNA, 498 bp, synthetic construct
  source 1..498: mol_type = other DNA
atggacttcgagtgccagtttgtgtgcgagctgaaggaactggcgccggttccggcgctgctgatccgtacccaaaccaccatgagcgagctgggtagcctgttcgaagcgggctaccacgatattctgcagctgctggcgggtcagggtaagagcccgagcggtccgccgtttgcgcgttattttggtatgagcgcgggcacctttgaggtggagttcggctttccggtggagggtggcgttgaaggtagcggccgtgtggttaccggtctgaccccgagcggcaaggcggcgagcagcctgtacatcggtccgtggggcgagattgaagcggtgtacgacgcgctgatgaaatgggttgacgataacggtttcgatctgagcggcgaggcgtacgaaatctatctggacaacccggcggaaaccgcgccggatcagctgcgtacccgtgttagcctgatgctgcacgagagcctggaacaccaccaccaccaccac

SEQ ID NO: 150 - protein, 166 aa, synthetic construct
MDFECQFVCELKELAPVPALLIRTQTTMSELGSLFEAGYHDILQLLAGQGKSPSGPPFARYFGMSAGTFEVEFGFPVEGGVEGSGRVVTGLTPSGKAASSLYIGPWGEIEAVYDALMKWVDDNGFDLSGEAYEIYLDNPAETAPDQLRTRVSLMLHESLEHHHHHH

SEQ ID NO: 151 - DNA, 495 bp, synthetic construct
  source 1..495: mol_type = other DNA
atggagtaccagctgcagcaactggcgagcctgaccctggtgggtatcaaggagacctatgaaaacggccgtcaagcgtatcaacacctgaggggtttctatcagcgttgcattcaagagggcgttattgcggacctgcagctgaagaacaacggtgatctggcgggtatcctgggcctgtgcattccggaactggacggtaaaatgagctatatgatcgcggtgaccggcgacaacagcgcggacatcgcgaagtacgatgtggttaccctggcgagcagcaaatatatggtttttgaagcgcaaggtgcgttggggaaggcgatttttcaaaaacctgaggaagtgcaccactacattcaccagtatcaagcgaacaccgttaaaagcgcgccgttcttttttctgcagcaagacggtgatacgaccagcgagaagtatatcaccgaaatttggatgccggttaaaggcctggaacaccaccaccaccaccac

SEQ ID NO: 152 - protein, 165 aa, synthetic construct
MEYQLQQLASLTLVGIKETYENGRQAYQHLRGFYQRCIQEGVIADLQLKNNGDLAGILGLCIPELDGKMSYMIAVTGDNSADIAKYDVVTLASSKYMVFEAQGALGKAIFQKPEEVHHYIHQYQANTVKSAPFFFLQQDGDTTSEKYITEIWMPVKGLEHHHHHH

SEQ ID NO: 153 - protein, 166 aa, synthetic construct
MDFECQFVCELKELAPVPALLIRTQTTMSELGSLFEAGYHDILQLLAGQGKSPSGPPFARYFGMSAGTFEVEFGFPVEGGVEGSGRVVTGLTPSGKAASSLYIGPYGEIEAVYDALMKWVDDNGFDLSGEAYEIYLDNPAETAPDQLRTRVSLMLHESLEHHHHHH

SEQ ID NO: 154 - protein, 165 aa, synthetic construct
MEYQLQQLASLTLVGIKETYENGRQAQQHIAGFWQRCYQEGVIADLQLKNNGDLAGILGLCIPELDGKMSYMIAVTGDNSADIAKYDVITLASSKYMVFEAQGAVPKAVQQKMEEVHHYIHQYQANTVKSAPFFELYQDGDTTSEKYITEIWMPVKGLEHHHHHH

SEQ ID NO: 155 - DNA, 12 bp, synthetic construct
  source 1..12: mol_type = other DNA
cagagcaaggac

SEQ ID NO: 156 - DNA, 45 bp, synthetic construct
  source 1..45: mol_type = other DNA
ggcggtggcggcgcgggcagagcaaggacgcggcggatcccactc

SEQ ID NO: 157 - DNA, 45 bp, synthetic construct
  source 1..45: mol_type = other DNA
tcgcctgtcctccgagccagtcgctgacagccgcggcgccgcgag

SEQ ID NO: 158 - DNA, 45 bp, synthetic construct
  source 1..45: mol_type = other DNA
gagtggccattcgacgacagtgtggtgtaaaggaattcattagcc

SEQ ID NO: 159 - DNA, 45 bp, synthetic construct
  source 1..45: mol_type = other DNA
ggggtttcctgcttcaacagtgcttggacggaacccggcgctcgt

SEQ ID NO: 160 - DNA, 45 bp, synthetic construct
  source 1..45: mol_type = other DNA
tctgtctcttgcttcaacagtgtttggacggaacagatccgggga

SEQ ID NO: 161 - DNA, 45 bp, synthetic construct
  source 1..45: mol_type = other DNA
ccgcctgtcctccgagccagtcgctgacagccgcggcgccgcgag

SEQ ID NO: 162 - DNA, 45 bp, synthetic construct; identical to SEQ ID NO: 159
  source 1..45: mol_type = other DNA

SEQ ID NO: 163 - DNA, 45 bp, synthetic construct; identical to SEQ ID NO: 160
  source 1..45: mol_type = other DNA

SEQ ID NO: 164 - RNA, 214 nt, synthetic construct
  source 1..214: mol_type = other RNA
cctcccctggggaggctcgcgttcccgctgctcgcgcctgcgccgcccgccggcctcaggaacgcgccctcttcgccggcgcgcgccctcgcagtcaccgccacccaacagctccggcaccaacagcagcgccgctgccaccgcccaccttctgccgccgccaccactgccaccttctcctcctccgctgtcctctccgtcctcgcctctgtcg

SEQ ID NO: 165 - DNA, 39 bp, synthetic construct
  source 1..39: mol_type = other DNA
gtggcggcgcgggcagagcaaggacgcggcggatcccac

SEQ ID NO: 166 - DNA, 40 bp, synthetic construct
  source 1..40: mol_type = other DNA
tttcctgcttcaacagtgcttggacggaacccggcgctcg

SEQ ID NO: 167 - DNA, 40 bp, synthetic construct
  source 1..40: mol_type = other DNA
gcgcgcgccctcgcagtcaccgccacccaacagctccggc

SEQ ID NO: 168 - DNA, 40 bp, synthetic construct
  source 1..40: mol_type = other DNA
gcgcgcgccctcgcagtcaccgccacccaccagctccggc

SEQ ID NO: 169 - DNA, 40 bp, synthetic construct; identical to SEQ ID NO: 168
  source 1..40: mol_type = other DNA

SEQ ID NO: 170 - DNA, 40 bp, synthetic construct
  source 1..40: mol_type = other DNA
ccgcgcgcgctctcagtctccgccacccaccagctccagc

SEQ ID NO: 171 - DNA, 40 bp, synthetic construct
  source 1..40: mol_type = other DNA
gcgcgcgcgctcacagtctccgccacccaccagctccagc

SEQ ID NO: 172 - DNA, 149 bp, synthetic construct
  source 1..149: mol_type = other DNA
taatacgactcactatagggggcaccaacagcagcgccgctgccaccgcccaccttctgccgccgccaccacagccaccttctcctcctccgctgtcctctcccgtcctcgcctctgtcgactatcaggtgaactttgaaccaggatgg

SEQ ID NO: 173 - protein, 130 aa, synthetic construct
LNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFE

SEQ ID NO: 174 - protein, 129 aa, synthetic construct
LNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFE

SEQ ID NO: 175 - protein, 459 aa, synthetic construct
MGHHHHHHGSDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGGGGGSENLYFQGASEFKLMPPIRNVTVAYKFHMGLNRETGRLSLESNSISPKLRSIAGYYDNPGMVPPDKCRCAVGSILSEGEESPSPELIDLYQKFGFKVFSFPAPSHVVTATFPYTTELSRDLATRRVHPALDTYIKERKLCAYPRLEIYQRDQIHFMCPLAVDTGNGSQMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNDDLILSCCLHYCKDKAKDFMPASKEEPIRLLREVVLLTDDRNLRVKALTRNVPVRDIPAFLTWAQVG

SEQ ID NO: 176 - protein, 872 aa, synthetic construct
MGTFFYGGSRGKRNNFKTEEYGGGGSMGRSRLLEDFRNNRYPNLQLREIAGHIMEFSQDQHGSRFIRLKLERATPAERQLVFNEILQAAYQLMVDVFGSYVIEKFFEFGSLEQKLALAERIRGHVLSLALQMYGSRVIRKALEFIPSDQQNEMVRELDGHVLKCVKDQNGSHVVEKCIECVQPQSLQFIIDAFKGQVFALSTHPYGCRVIQRILEHCLPDQTLPILEELHQHTEQLVQDQYGSYVIRHVLEHGRPEDKSKIVAEIRGNVLVLSQHKFASNVVEKCVTHASRTERAVLIDEVCTMNDGPHSALYTMMKDQYACYVVQKMIDVAEPGQRKIVMHKIRPHIATLRKYTYGKHILAKLEKYYMKNGVDLGGGGGSGGGGSGGGGSMPPIRNVTVAYKFHMGLYGETRRLYSESRSISPKLRSIAGYYDNPRMVPPDKCRCAVGSILSEGEESPSPELIDLYQKFGFKVFSFPAPSHVVTATFPYTTELSGALATRRVHPSLDTYIKERKLCAYPRLEIYQRDQIHFMCPLAGGGGSGGGGSGGGGSQMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNDDLILSCCLHYCKDKAKDFMPASKEEPIRLLREVVLLTDDRNLRVKALTRNVPVRDIPAFLTWAQVGGGGGSGGGGSLAEAKVLANRELDKYGVSDFYKRLINKAKTVEGVEALKLHILAALPGGGGSGGGGSLAEAKVLANRELDKYGVSDFYKRLINKAKTVEGVEALKLHILAALPGGGGSENLYFQGASEFKLGSHHHHHH

SEQ ID NO: 177 - protein, 872 aa, synthetic construct
MGTFFYGGSRGKRNNFKTEEYGGGGSMGRSRLLEDFRNNRYPNLQLREIAGHIMEFSQDQHGSRFIRLKLERATPAERQLVFNEILQAAYQLMVDVFGSYVIEKFFEFGSLEQKLALAERIRGHVLSLALQMYGSRVIRKALEFIPSDQQNEMVRELDGHVLKCVKDQNGSHVVEKCIECVQPQSLQFIIDAFKGQVFALSTHPYGCRVIQRILEHCLPDQTLPILEELHQHTEQLVQDQYGSYVIRHVLEHGRPEDKSKIVAEIRGNVLVLSQHKFASNVVEKCVTHASRTERAVLIDEVCTMNDGPHSALYTMMKDQYACYVVQKMIDVAEPGQRKIVMHKIRPHIATLRKYTYGKHILAKLEKYYMKNGVDLGGGGGSGGGGSGGGGSMPPIRNVTVAYKFHMGLASETGRLASESGSISPKLRSIASYYDNPSMVPPDKCRCAVGSILSEGEESPSPELIDLYQKFGFKVFSFPAPSHVVTATFPYTTQLSRSLATRRVHPALDTYIKERKLCAYPRLEIYQNDQIHFMCPLAGGGGSGGGGSGGGGSQMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNDDLILSCCLHYCKDKAKDFMPASKEEPIRLLREVVLLTDDRNLRVKALTRNVPVRDIPAFLTWAQVGGGGGSGGGGSLAEAKVLANRELDKYGVSDFYKRLINKAKTVEGVEALKLHILAALPGGGGSGGGGSLAEAKVLANRELDKYGVSDFYKRLINKAKTVEGVEALKLHILAALPGGGGSENLYFQGASEFKLGSHHHHHH

SEQ ID NO: 178 - protein, 395 aa, synthetic construct
MGHHHHHHPPIRNVTVAYKFHMGLDHETRRLYRESDSISPKLRSIASYYDNPNMVPPDKCRCAVGSILSEGEESPSPELIDLYQKFGFKVFSFPAPSHVVTATFPYTTGLSEDLATRRVHPALDTYIKERKLCAYPRLEIYQEDQIHFMCPLAGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 395 aa, synthetic construct
MGHHHHHHPPIRNVTVAYKFHMGLWMETGRLARESVSISPKLRSIAVYYDNPHMVPPDKCRCAVGSILSEGEESPSPELIDLYQKFGFKVFSFPAPSHVVTATFPYTTMLSTLLATRRVHPALDTYIKERKLCAYPRLEIYQNDQIHFMCPLAGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - protein, 872 aa, synthetic construct
MGTFFYGGSRGKRNNFKTEEYGGGGSMGRSRLLEDFRNNRYPNLQLREIAGHIMEFSQDQHGSRFIRLKLERATPAERQLVFNEILQAAYQLMVDVFGSYVIEKFFEFGSLEQKLALAERIRGHVLSLALQMYGSRVIRKALEFIPSDQQNEMVRELDGHVLKCVKDQNGSHVVRKCIECVQPQSLQFIIDAFKGQVFALSTHPYGSRVIERILEHCLPDQTLPILEELHQHTEQLVQDQYGNYVIQHVLEHGRPEDKSKIVAEIRGNVLVLSQHKFASYVVRKCVTHASRTERAVLIDEVCTMNDGPHSALYTMMKDQYANYVVQKMIDVAEPGQRKIVMHKIRPHIATLRKYTYGKHILAKLEKYYMKNGVDLGGGGGSGGGGSGGGGSMPPIRNVTVAYKFHMGLSEETSRLNRESRSISPKLRSIADYYDNPKMVPPDKCRCAVGSILSEGEESPSPELIDLYQKFGFKVFSFPAPSHVVTATFPYTTQLSNALATRRVHPALDTYIKERKLCAYPRLEIYQRDQIHFMCPLAGGGGSGGGGSGGGGSQMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNDDLILSCCLHYCKDKAKDFMPASKEEPIRLLREVVLLTDDRNLRVKALTRNVPVRDIPAFLTWAQVGGGGGSGGGGSLAEAKVLANRELDKYGVSDFYKRLINKAKTVEGVEALKLHILAALPGGGGSGGGGSLAEAKVLANRELDKYGVSDFYKRLINKAKTVEGVEALKLHILAALPGGGGSENLYFQGASEFKLGSHHHHHH

(19) United States
(12) Patent Application Publication
(10) Pub. No.: US 2026/0184757 A1
(43) Pub. Date: Jul. 2, 2026

(54) Engineered Therapeutic PROTEIMER Compositions and Related Methods

(71) Applicant: CrossLife Technologies Inc., Carlsbad, CA (US)

(72) Inventors: HyunDae Cho, Carlsbad, CA (US); Hannah K. Cho, Carlsbad, CA (US)

(21) Appl. No.: 19/375,167

(22) Filed: Oct. 30, 2025

Related U.S. Application Data
(60) Provisional application No. 63/713,768, filed on Oct. 30, 2024.

(51) Int. Cl. C07K 14/705; A61P 25/28; A61P 35/00; C07K 14/47

(52) U.S. Cl. CPC C07K 14/70503; A61P 25/28; A61P 35/00; C07K 14/4711; C07K 2319/30; C07K 2319/80

(57) ABSTRACT

The present invention pertains to the field of protein engineering, molecular imaging, molecular diagnostics, and biopharmaceutics. Provided herein are six unique Proteimer protein scaffolds (TEX-S2/S3, TEX-S4, YTHDF3, PUM, DARPin, and Aca2) that demonstrate specificity and affinity comparable to antibodies across a broad range of therapeutic targets. Methods and protocols are provided for generating non-native protein aptamers, Proteimers, which are capable of binding to a diverse set of targets, including RNA, DNA, proteins, post-translational modifications, peptides, small molecules, and prosthetic groups. This platform supports the creation of biotherapeutic aptamers from Proteimer scaffolds for the treatment of various diseases.

### CROSS-REFERENCE TO RELATED APPLICATION(S)

This application is a National Stage of International Application No. 63/713,768, filed Oct. 30, 2024, the disclosure of which is hereby incorporated by reference in its entirety.

### FIELD OF INVENTION

The present invention is related to the field of protein engineering, molecular imaging, molecular diagnostics, and biopharmaceutics.

### REFERENCE TO AN ELECTRONIC SEQUENCE LISTING

The contents of the electronic sequence listing (Sequence_Listing-128617-0002UT01.xml; Size: 188,831 bytes; and Date of Creation: Oct. 30, 2025) is herein incorporated by reference in its entirety.

### BACKGROUND

Biotherapeutics is a growing field that has revolutionized modern medicine. The growth has been stimulated by antibody technologies that can target a wide range of conditions such as autoimmune/autoinflammatory diseases and cancer.

Engineered protein scaffolds that bind with high specificity to target molecules are growing tools that have provided a powerful platform for creating novel biopharmaceutical technologies that can work similarly to monoclonal antibodies. Such technologies provide cost-effective and modular approaches to create aptamers that bind to biomarkers or therapeutic targets with high affinity. To date, several scaffolds have been engineered to create drug products for a wide range of diseases, many of which have entered late-stage clinical trials. The growth of these promising technologies has inspired the field of aptamer biopharmaceutical technologies. These aptamers are beneficial because of their high stability which allows for advance engineering that cannot be conducted on antibodies.

The screening and design of proteins have emerged as key avenues in therapeutic discovery, leveraging the vast diversity of natural protein structures, functions, and interactions. Although life-changing, antibody therapeutics remain costly to manufacture and the process of drug discovery is laborious. Therefore, improved methods for generating novel biotherapeutics are in high demand.

### SUMMARY

Provided herein are invention Proteimers (Protein-Based Aptamers) that capitalize on the diversity of natural protein, facilitating the high-throughput development of high-affinity binders targeting small molecules, proteins, and nucleic acids.

Accordingly, provided herein are the following items:
1. A non-native variant proteimer, relative to wild-type TEX264, comprising any combination of one up to all 8 of variant amino acid residues, wherein the variant amino acid residues correspond to residues 18, 19, 25, 26, 29, 102, 105 and 106 of TEX-S2 (SEQ ID NO:2).
2. The non-native variant proteimer of item 1, wherein the number of variant amino acid residues is selected from the group consisting of: 1, 2, 3, 4, 5, 6, 7, and 8.
3. A non-native variant proteimer, relative to wild-type TEX264, comprising any combination of one up to all 12 of variant amino acid residues, wherein the variant amino acid residues correspond to residues 18, 19, 22, 25, 26, 29, 40, 46, 102, 105, 106, and 136 of TEX-S3 (SEQ ID NO:4).
4. The non-native variant proteimer of item 3, wherein the number of variant amino acid residues is selected from the group consisting of: 1, 2, 3, 4, 5, 6, 7, 8, 9, 10, 11 and 12.
5 The variant proteimer of items 3-4, wherein the proteimer is selected from the group consisting of SEQ ID NOs: 16 (SNCA-B), 17 (SNCA-C), 21 (APPS3-5), 22 (APPS3-11), 24 (TEX-T1), 25 (TEX-T2), 27 (TEX-CD19-6), 28 (TEX CD19-8), and 30-110.
6. The variant proteimer of items 3-5, wherein the proteimer is selected from the group consisting of SEQ ID NOs: 16 (SNCA-B), 17 (SNCA-C), 21 (APPS3-5), 22 (APPS3-11), 24 (TEX-T1), 25 (TEX-T2), 27 (TEX-CD19-6), 28 (TEX CD19-8).
7. A non-native variant proteimer, relative to wild-type TEX264, comprising any combination of one up to all 12 of variant amino acid residues, wherein the variant amino acid residues correspond to residues 31, 32, 35, 38, 39, 42, 53, 59, 115, 118, 119, and 149 of TEX-S4 (SEQ ID NO:6).
8. The non-native variant proteimer of item 7, wherein the number of variant amino acid residues is selected from the group consisting of: 1, 2, 3, 4, 5, 6, 7, 8, 9, 10, 11 and 12.
9. A non-native variant proteimer, relative to wild-type YTHDF3, comprising any combination of one up to all 11 of variant amino acid residues, wherein the variant amino acid residues correspond to residues 40, 54, 55, 57, 84, 108, 113, 129, 147, 149, and 150 of YTHDF3 (SEQ ID NO:8).
10. The non-native variant proteimer of item 9, wherein the number of variant amino acid residues is selected from the group consisting of: 1, 2, 3, 4, 5, 6, 7, 8, 9, 10, and 11.
11. The variant proteimer of items 9-10, wherein the proteimer is selected from the group consisting of SED ID NOs: 124-138.
12. A non-native variant proteimer, relative to wild-type PUM, comprising any combination of one up to all 24 of variant amino acid residues, wherein the variant amino acid residues correspond to residues 37, 38, 41, 73, 74, 77, 109, 110, 113, 145, 146, 149, 181, 182, 185, 217, 218, 221, 253, 254, 257, 296, 297, and 300 of PUM (SEQ ID NO: 10).
13. The non-native variant proteimer of item 12, wherein the number of variant amino acid residues is selected from the group consisting of: 1, 2, 3, 4, 5, 6, 7, 8, 9, 10, 11, 12, 13, 14, 15, 16, 17, 18, 19, 20, 21, 22, 23, and 24.
14. A non-native variant proteimer, relative to wild-type DARPin, comprising any combination of one up to all 21 of variant amino acid residues, wherein the variant amino acid residues correspond to residues 32, 34, 35, 37, 45, 46, 58, 65, 67, 68, 70, 78, 79, 91, 98, 100, 101, 103, 111, 112, and 124 of DARPin (SEQ ID NO:12).
15. The non-native variant proteimer of item 14, wherein the number of variant amino acid residues is selected from the group consisting of: 1, 2, 3, 4, 5, 6, 7, 8, 9, 10, 11, 12, 13, 14, 15, 16, 17, 18, 19, 20, and 21.
16. A non-native variant proteimer, relative to wild-type Aca2, comprising any combination of one up to all 14 of variant amino acid residues, wherein the variant amino acid residues correspond to residues 28, 30, 31, 33, 34, 39, 45, 149, 151, 152, 154, 155, 160, and 166 of Aca2 (SEQ ID NO:14).
17. The non-native variant proteimer of item 16, wherein the number of variant amino acid residues is selected from the group consisting of: 1, 2, 3, 4, 5, 6, 7, 8, 9, 10, 11, 12, 13, and 14.
18. The variant proteimer of items 16-17, wherein the proteimer is selected from the group consisting of SED ID NOs: 141-148.
19. A non-native variant proteimer, relative to a wild-type nanobody, comprising any combination of one up to all 22 of variant amino acid residues, wherein the variant amino acid residues correspond to residues 28, 29, 30, 31, 32, 34, 35, 37, 56, 57, 58, 61, 103, 104, 105, 106, 107, 108, 109, 110, 111, and 112 of the nanobody (SEQ ID NO: 18).
20. The non-native variant proteimer of item 19, wherein the number of variant amino acid residues is selected from the group consisting of: 1, 2, 3, 4, 5, 6, 7, 8, 9, 10, 11, 12, 13, 14, 15, 16, 17, 18, 19, 20, 21, and 22.
21. The variant proteimer of items 19-20, wherein the proteimer is selected from the group consisting of SED ID NOs: 112-121.
22. A chimeric proteimer construct comprising,
   a proteimer moiety according to items 1-21; and
   a recombinant polypeptide moiety.
23. The chimeric proteimer of item 22, wherein the polypeptide moiety is selected from one or more of a proteolytic degradation moiety, an Fc-region moiety and/or another proteimer moiety according to items 1-21.
24. The chimeric proteimer of items 22-23, wherein the proteolytic degradation moieity is selected from proteases and/or nucleases.
25. The chimeric proteimer of items 22-24, wherein the nuclease moiety is an RNAse moiety selected from one or more of: RNase A, PIN, SMG6 PIN, RENT1/UPF1 PIN, viral PIN domains, RNase MRP, RelE toxin PIN, Cas13, Onconase, RNAse 2, RNAse 3, RNAse 5, Barnase, Binase and/or pinard.
26. The chimeric proteimer of item 25, wherein the chimeric proteimer binds and cleaves a specific RNA target.
27. The chimeric proteimer of items 25-26, wherein the RNA target comprises an Internal Ribosomal Entry Site (IRES) and/or an Iron-Responsive Element (IRE).
28. The chimeric proteimer of items 25-27, wherein the RNA target is a 5′ UTR region of an mRNA transcript coding for a protein selected from: c-myc, APP, tau, SNCA (Alpha-synuclein), ARID5A, A323M, Ferritin, and C-jun.
29. The chimeric proteimer of items 22-28, further comprising one or more of a pumilo (PUM) domain, a blood-brain barrier transcytosis domain, and a half-life extending domain.
30. The chimeric proteimer of items 29, wherein the pumilo (PUM) domain is the proteimer of item 12, wherein the blood-brain barrier transcytosis domain is selected from Angiopep-2 peptide, HIV-1 Tat peptide, Rabies Virus Glycoprotein (RVG29), RDP-shuttle peptide, and/or Melanotransferrin-derived peptides; and wherein the half-life extending domain is selected from a transferrin receptor targeting protein domain, an FcBP domain, PEGylation, XTEN, PASylation and/or an albumin binding domain.
31. The chimeric proteimer of items 22-30, wherein pumilo domain is amino acids 27-376 of SEQ ID NO: 176; the blood-brain barrier transcytosis domain is Angiopep-2 peptide; the half-life extending domain is an albumin binding domain.
32. The chimeric proteimer of items 22-31, wherein the proteimer moiety is selected from the group consisting of SEQ ID NOs: 16, 17, 19, 21, 22, 24, 25, 30-38, 112-115, 124-138, and 141-148.
33. The chimeric proteimer of items 22-24, wherein the protease moiety is obtained from one or more of: TRIM21, VHL, CRBN, MDM2, RNF4, FBW7, CHIP, FBXW7, Subtilisin, Trypsin, TEV protease, Granzyme B, Caspases, Papain, Cathepsins, Calpain, MMPs, ADAMs, HIV protease, and/or Cathepsin D.
34. The chimeric proteimer of items 33, wherein the chimeric proteimer binds and cleaves a specific protein target.
35. The chimeric proteimer of items 22-23, wherein the polypeptide moiety is an Fc-region moiety and the proteimer moiety is selected from the group consisting of SEQ ID NOs: 27, 28, 40-110, and 116-121.
36. The chimeric proteimer of item 35, which is selected from:
   TEX-CD19_8: Fc-region (SEQ ID NO: 178); and
   TEX-CD19_6: Fc-region (SEQ ID NO:179).
37. A library of non-native TEX264 variant proteimers comprising non-native variant proteimers having any combination of one up to all 8 of variant amino acids, relative to wild-type TEX264, among all variant-positions in the polpeptide set forth in SEQ ID NO: 2; wherein the variant positions correspond to amino acid residues 18, 19, 25, 26, 29, 102, 105 and 106 of TEX-S2 (SEQ ID NO:2).
38. A library of non-native TEX264 variant proteimers comprising non-native variant proteimers having any combination of one up to all 12 of variant amino acids, relative to wild-type TEX264, among all variant-positions in the polpeptide set forth in SEQ ID NO: 4; wherein the variant positions correspond to amino acid residues 18, 19, 22, 25, 26, 29, 40, 46, 102, 105, 106, and 136 of TEX-S3 (SEQ ID NO:4).
39. A library of non-native TEX264 variant proteimers comprising non-native variant proteimers having any combination of one up to all 12 of variant amino acids, relative to wild-type TEX264, among all variant-positions in the polpeptide set forth in SEQ ID NO: 6; wherein the variant positions correspond to amino acid residues 31, 32, 35, 38, 39, 42, 53, 59, 115, 118, 119, and 149 of TEX-S4 (SEQ ID NO:6).
40. A library of non-native YTHDF3 variant proteimers comprising non-native variant proteimers having any combination of one up to all 11 of variant amino acids, relative to wild-type YTHDF3, among all variant-positions in the polpeptide set forth in SEQ ID NO: 8; wherein the variant positions correspond to amino acid residues 40, 54, 55, 57, 84, 108, 113, 129, 147, 149, and 150 of YTHDF3 (SEQ ID NO:8).
41. A library of non-native PUM variant proteimers comprising non-native variant proteimers having any combination of one up to all 24 of variant amino acids, relative to wild-type PUM, among all variant-positions in the polpeptide set forth in SEQ ID NO: 10; wherein the variant positions correspond to amino acid residues 37, 38, 41, 73, 74, 77, 109, 110, 113, 145, 146, 149, 181, 182, 185, 217, 218, 221, 253, 254, 257, 296, 297, and 300 of PUM (SEQ ID NO:10).
42. A library of non-native DARPin variant proteimers comprising non-native variant proteimers having any combination of one up to all 21 of variant amino acids, relative to wild-type DARPin, among all variant-positions in the polpeptide set forth in SEQ ID NO: 12; wherein the variant positions correspond to amino acid residues 32, 34, 35, 37, 45, 46, 58, 65, 67, 68, 70, 78, 79, 91, 98, 100, 101, 103, 111, 112, and 124 of DARPin (SEQ ID NO:12).
43. A library of non-native Aca2 variant proteimers comprising non-native variant proteimers having any combination of one up to all 14 of variant amino acids, relative to wild-type Aca2, among all variant-positions in the polpeptide set forth in SEQ ID NO: 14; wherein the variant positions correspond to amino acid residues 28, 30, 31, 33, 34, 39, 45, 149, 151, 152, 154, 155, 160, and 166 of Aca2 (SEQ ID NO:14).
44. A library of non-native nanobody variant proteimers comprising non-native variant proteimers having any combination of one up to all 22 of variant amino acids, relative to wild-type nanoboy, among all variant-positions in the polpeptide set forth in SEQ ID NO: 18; wherein the variant positions correspond to amino acid residues 28, 29, 30, 31, 32, 34, 35, 37, 56, 57, 58, 61, 103, 104, 105, 106, 107, 108, 109, 110, 111, and 112 of the nanobody (SEQ ID NO:18).
45. A chimeric multivalent proteimer construct comprising,
   a first proteimer moiety according to items 1-21; and
   a second proteimer moiety according to items 1-21.
46. The chimeric multivalent proteimer of item 45, wherein the multivalent proteimer further comprises a half-life extending domain.
47. The chimeric multivalent proteimer of item 45-46, wherein the half-life extending domain is selected from a transferrin receptor targeting protein domain, an FcRn-binding peptide domain, Fc-region, PEGylation, XTEN, PASylation and/or an albumin binding domain.
48. The chimeric multivalent proteimer of item 45-47, wherein the half-life extending domain is an Fc-region.
49. The chimeric multivalent proteimer of item 45-48, wherein the first and second proteimer moiety binds to a protein selected from the group consisting of: CD19, CD3, CD33, CD37, Hur, CD20, CD22, Bcl2, Her2, CD37.
50. The chimeric multivalent proteimer of item 45-49, wherein the first and second proteimer moiety are selected from any combination of 2 or more of SEQ ID NOs: 27, 28, 47-62, 73-110, and 116-121.
51. The chimeric multivalent proteimer of item 45-50, wherein the first and second proteimer moiety are selected from any combination of 2 or more of SEQ ID NOs: 27, 28, 47, 48, 55-57, 93-103 and 119, wherein the first protiemer moiety binds to CD19 and the second proteimer moiety binds to CD20.
52. A method of treating cancer, comprising administering to a patient in need thereof, a chimeric proteimer construct of items 22 or 45, wherein the proteimer moiety is selected from one or more of SEQ ID NOs: 27, 28, 47-62, 73-110, 116-121, 131-138, and 141-148.
53. The method item 52, wherein the proteimer moiety binds to a protein selected from the group consisting of: CD19, CD3, CD33, CD37, Hur, CD20, CD22, Bcl2, Her2, CD37.
54. The method items 52-53, wherein the proteimer moiety binds to CD19.
55. The method items 52-54, wherein the proteimer moiety is SEQ ID NO: 27 and/or SEQ ID NO:28.
56. The method items 52-55, wherein the chimeric proteimer construct is SEQ ID NO: 178 (CD19_8-Fc) and/or SEQ ID NO: 179 (CD19_6-Fc).
57. The method item 52, wherein the proteimer moiety binds to a 5′ UTR mRNA target coding for a protein selected from the group consisting of: cMyc-A323M and ARID5A.
58. The method items 52 and 57, wherein the proteimer moiety is selected from one or more of SEQ ID NOs: 131-138 and 141-148.
59. A method of treating Alzheimer's disease, comprising administering to a patient in need thereof, a chimeric proteimer construct of items 22-32, wherein the proteimer moiety is selected from one or more of SEQ ID NOs: 21, 22, 24, 25, 32-38, 63, 64, 113-115, and 124-130.
60. The method item 59, wherein the proteimer moiety binds to a 5′ UTR mRNA target coding for a protein selected from the group consisting of: APP, Tau, Ferritin, and Tau60.
61. The method items 59-60, wherein the proteimer moiety binds to a 5′ UTR mRNA target coding for APP.
62. The method items 59-61, wherein the proteimer moiety is SEQ ID NO: 21 and/or SEQ ID NO:22.
63. The method items 59-62, wherein the chimeric proteimer construct is SEQ ID NO: 176 (ProAPPS3-5) and/or SEQ ID NO:177 (ProAPPS3-11).
64. The method items 59-60, wherein the proteimer moiety binds to a 5′ UTR mRNA target coding for Tau.
65. The method items 59-60 and 64, wherein the proteimer moiety is SEQ ID NO: 24.
66. The method items 59-62, wherein the chimeric proteimer construct is SEQ ID NO: 180 (ProTEX-T1).
67. A method of treating Parkinson's disease, comprising administering to a patient in need thereof, a chimeric proteimer construct of items 22-32, wherein the proteimer moiety is selected from one or more of SEQ ID NOs: 16, 17, 19, 30, 31, and 112.
68. The method item 67, wherein the proteimer moiety binds to a 5′ UTR mRNA target coding for alpha-Synuclein.
69. A method of treating pain or a neurological disorder, comprising administering to a patient in need thereof, a chimeric proteimer construct of items 22-32, wherein the proteimer moiety is selected from one or more of SEQ ID NOs: 65-72.
70. A method of treating opiod addiction, comprising administering to a patient in need thereof, a chimeric proteimer construct of items 22-32, wherein the proteimer moiety is selected from one or more of SEQ ID NOs: 39-44.
71. A method of inhibiting SARS-Cov-2 virus interacting or binding with a host cell, comprising administering to a patient in need thereof, a chimeric proteimer construct of items 22-32, wherein the proteimer moiety is selected from one or more of SEQ ID NOs: 150 and/or 152, and wherein the proteimer moiety is capable of binding to SARS-COV2 spike protein and is capable of inhibiting the interaction betweens SARS-Cov-2 and human ACE2 receptor.
72. A method for eliminating or decreasing a toxicity of a chemotherapeutic agent, said method comprising administering to a patient in need thereof, a chimeric proteimer construct of items 22-32, wherein proteimer moiety binds to said chemotherapeutic agent having therapeutic activity, wherein the toxicity is eliminated or decreased upon binding by the proteimer moiety.
73. The method of item 72, wherein the chimeric proteimer construct further comprises an FcRn-binding peptide (FcBp).
74. The method of items 72-73, wherein the chemotherapeutic agent is Daunorubicin.
75. The method of items 72-74, wherein the the proteimer moiety is SEQ ID NO:150.
76. A method of making a non-native variant proteimer of items 1-21, comprising expressing nucleic acid encoding said proteimer in a suitable host cell; and isolating the expressed protein.

The invention Proteimer libraries were initially screened in silico using tools, such as AlphaFold3 to assess stability and inform design (FIG. 1). These libraries enhance the native binding modes of each scaffold protein by introducing targeted mutations at key amino acid positions to modulate binding to specific targets (FIG. 1). Six unique Proteimer protein scaffolds (TEX-S2/S3, TEX-S4, YTHDF3, PUM, DARPin, and Aca2) have been developed for these libraries, permitting the achievement of specificity and affinity comparable to antibodies and antibody mimetics across a wide range of therapeutic targets (FIG. 1).

Gyrl-like proteins (TEX-S2/S3, TEX-S4) are valuable candidates for the development of biotherapeutic aptamer technologies. Their ability to bind organic molecules of varying shape, size and chemical properties have provided a stable protein scaffold template for engineering novel aptamers with special properties. Examples of Gyrl-like proteins that were modified through rational engineering or by phage display to generate Proteimers that function as fluorescence enhancers or quenchers of organic dyes, enzymes, aptamers are set forth U.S. Pat. No. 12,104,201; which is incorporated herein by reference in its entirety for all purposes. For the invention engineering of Proteimers (protein aptamers), target molecules have been selected herein to include proteins with post-translational modification groups and short exposed peptide epitopes. Provided herein are novel non-native properties of invention Proteimers (protein aptamers) and examples of their application to biopharmaceutics. The invention protocol utilized phage display and selection to identify Proteimers that bind to target proteins immobilized onto bead surfaces.

Also provided herein are isolated Proteimers (protein aptamers) that bind to the spike protein receptor binding domain (spike-RBD) of SARS-COV-2 coronavirus, the causative agent of the global COVID19 pandemic. Proof has been demonstrated that rationally-designed Proteimers or those engineered from mutant libraries can bind to a spike protein with similar affinity to monoclonal antibodies used for the therapeutic treatment SARS-COV-2. Furthermore, it has been demonstrated that binding by Proteimer can inhibit interactions between the spike protein and host receptor angiotensin-converting enzyme 2 (ACE2). Also provided is an innovative method for rapid identification of inhibitors of spike proteins using stable engineered Proteimers as bait.

The invention broadly describes a novel and versatile approach to convert protein scaffolds, such as these six different protein scaffolds (TEX-S2/S3, TEX-S4, YTHDF3, PUM, DARPin, and Aca2), into powerful aptamer therapeutics for various diseases.

The present invention relates to the field of protein engineering and biotherapeutics. More particularly, provided herein are additional methods to design non-native Proteimers (TEX-S2/S3, TEX-S4, YTHDF3, PUM, DARPin, and Aca2), whereby the non-native proteins are referred to as Proteimers. Also provided herein are Proteimers that can bind to the receptor binding domain of SARS-COV-2 Spike protein in a complex that inhibits the interaction of human ACE2 receptor.

The invention presents additional methods related to patent filing (U.S. Pat. No. 12,104,201) that describes an efficient strategy and protocol for creating non-native protein aptamers that bind organic compounds. Aptamers are created through rational mutagenesis or through selection from phage display libraries. Combined the extended protocol describes a broad procedure to identify Proteimers for biotechnological uses. The invention demonstrates methods to create Proteimer variants that bind to protein targets. These “antibody-like” aptamers can be further developed for biopharmaceutical uses.

The invention Proteimers library has been extended beyond the Gyrl-like domain, developing six unique protein scaffolds (TEX-S2/S3, TEX-S4, YTHDF3, PUM, DARPin, and Aca2) that achieve specificity and affinity comparable to antibodies across a wide range of therapeutic targets. Additionally, provided herein are methods and protocols for generating non-native protein aptamers, termed Proteimers, capable of binding an extended set of targets, including RNA, DNA, proteins, post-translational modifications, peptides, small molecules, and prosthetic groups. This platform enables the creation of biotherapeutic aptamers from Proteimer scaffolds for treating various diseases.

This invention demonstrates methods to design rational variants that can bind protein targets with high affinity. Provided herein is CTR107 Y106W with sequence defined in FIG. 12A (SEQ ID NO:8) that can bind Spike RBD with low nanomolar affinity. Additionally, provided herein are methods to identify Proteimer variants from libraries that can bind protein targets with high affinity. This invention demonstrates the discovery of a unique Proteimer designated SAV HS with its sequence described in FIG. 12A (SEQ ID NO: 9) that can bind Spike RBD with low nanomolar affinity. The methods used herein can be extended to identify Proteimers that can bind any protein target of interests. Overall, this invention describes methods for creating novel biotherapeutic aptamers from the Proteimer family which includes six different unique protein scaffolds (TEX-S2/S3, TEX-S4, YTHDF3, PUM, DARPin, and Aca2).

Also provided herein are extended protocols for engineering Proteimer aptamers to recognize novel targets that include protein, peptides, post-translational modifications and prosthetic groups. Also provided are methods for screening for inhibitors of SARS-COV-2 spike protein; and methods for engineering therapeutic Proteimers (protein aptamers) for targeting new strains of SARS-COV-2 or various strains of coronaviruses. Also contemplated herein are methods and protocols for engineering non-native Proteimers (protein aptamers) for biopharmaceutical and therapeutic uses as medicine to treat a broad range of diseases.

Also provided herein is a method for eliminating or decreasing a toxicity of a chemotherapeutic agent, said method comprising administering an invention Proteimer that binds to said chemotherapeutic agent having therapeutic activity, wherein the toxicity is eliminated or decreased (e.g., squelched) upon binding by the Proteimer. In certain embodiments, the Proteimer further comprises an FcRn-binding peptide (FcBp). In one embodiment, the chemotherapeutic agent is Daunorubicin. In another embodiment, the Proteimer is CTR Y106W.

Accordingly, the Proteimers provided herein are contemplated for use as a candidate small molecule binder (Daunorubicin binding Proteimer or others) to block toxicity of chemotherapeutic molecule. In certain embodiments, the candidate small molecule binder (invention Proteimer) will be added a couple hrs/days post chemotherapy to offset the toxic effects of the drug on non-cancerous, healthy cells.

In certain embodiments, with a trafficking FcBp peptide (FcRn-binding peptide) is added to the candidate chemotherapeutic agent-biding invention Proteimer (e.g., Daunorubicin binding Proteimer or the like), this permits recycling of the small molecule binder. In a particular embodiment, the chemotherapeutic drug and small molecule binders will be packaged within a liposome containing proteins that target cancer cells/leukocytes.

### BRIEF DESCRIPTION OF THE DRAWINGS

FIG. 1. Alphafold3 predictions of the Proteimer library constructs. Cartoon representations are shown for the FIG. 1A) TEX-S2/S3, FIG. 1B) TEX-S4, FIG. 1C) YTHDF3, FIG. 1D) PUM, FIG. 1 E) DARPin, and FIG. 1F) Aca2 Alphafold3 predicted structures associated with each Proteimer library design. Amino acid positions selected for mutation in each library are shaded.

FIG. 2. The TEX-S2 Proteimer binding site combinatorial mutant library encompasses a diverse range of nucleotide and amino acid sequences, designed to explore variations within the binding site for enhanced specificity and affinity toward target proteins.

FIG. 3. The TEX-S3 Proteimer binding site combinatorial mutant library encompasses a diverse range of nucleotide and amino acid sequences, designed to explore variations within the binding site for enhanced specificity and affinity toward target proteins.

FIG. 4. The TEX-S4 Proteimer binding site combinatorial mutant library encompasses a diverse range of nucleotide and amino acid sequences, designed to explore variations within the binding site for enhanced specificity and affinity toward target proteins.

FIG. 5. The YTHDF3 Proteimer binding site combinatorial mutant library encompasses a diverse range of nucleotide and amino acid sequences, designed to explore variations within the binding site for enhanced specificity and affinity toward target proteins.

FIG. 6. The PUM Proteimer binding site combinatorial mutant library encompasses a diverse range of nucleotide and amino acid sequences, designed to explore variations within the binding site for enhanced specificity and affinity toward target proteins.

FIG. 7. The DARPin Proteimer binding site combinatorial mutant library encompasses a diverse range of nucleotide and amino acid sequences, designed to explore variations within the binding site for enhanced specificity and affinity toward target proteins.

FIG. 8. The Aca2 Proteimer binding site combinatorial mutant library encompasses a diverse range of nucleotide and amino acid sequences, designed to explore variations within the binding site for enhanced specificity and affinity toward target proteins.

FIG. 9. Examples of multivalent Proteimer constructs. FIG. 9A) Example construct showing TEX-S4 and PUM Proteimers designed to target a particular RNA fused to an RNase domain to enable proximity induced target degradation. FIG. 9B) Example construct showing a TEX-S4 Proteimer, designed to target a protein, fused to a Protease domain to enable proximity induced degradation.

FIG. 10. Examples of RNA targets. FIG. 10A) Alignment of human and mouse APP 5′UTRs with human PrP 5′UTR sequences relative to the L- and H-ferritin iron-responsive elements (IREs) (SEQ ID NOs: 155-163). Relative alignment of the sequences that encode the 5′UTR specific IRE-like stem loops in human APP mRNA, PrP mRNA, SNCA mRNA, and the L- and H-ferritin mRNAs. The L- and H-mRNAs encode canonical IRE RNA stem loops whereas the APP IRE in non-canonical although fully iron responsive. The a-synuclein IRE (SNCA IRE) represents a non-canonical IRE traversing the central splice junction of exon-1 and exon-2 (the CAGUGN loop/splice site sequences) of SNCA mRNA. Typical IRE stem loops fold to exhibit an apical AGU pseudotriloop which is at the apex of the H-ferritin and SNCA IREs relative to an analogous AGA from the IRE-like stem loop encoded by APP mRNA. FIG. 10B) The predicted secondary structure of the tau 5′UTR mRNA (SEQ ID NO:164). RNAFold WebServer was used to predict folding of the RNA structure. FIG. 10C) The 5′UTR of tau mRNA encodes a significant IRE homology. Sequence alignments of the 5′ UTR regions encoding IRE-like stem loops in tau from humans, Trachypithecus, gorillas, mice, and rats reveal similarities when compared to the 5′ UTR mRNAs of human APP, Alpha-synuclein, and H-ferritin (SEQ ID NOs: 165-171).

FIG. 11. Schematic representation of the biopanning strategy and library design. FIG. 11A) Summary of the biopanning cycle. (1) Upon library amplification, (2) phages were purified from the lysate. (3) Each phage library was incubated with biotinylated 5′UTR of SNCA mRNA containing stem-loops. (4) Bound phages were enriched with streptavidin-conjugated magnetic beads and (6) amplified for the next round of biopanning. (5) Unbound phages were washed away. (7) Candidates were determined by sequencing single phage plaques and analyzing for converging sequences. Predicted structures are shown for FIG. 11B) TEXS3 Library and FIG. 11C) Nanobody Library with variable amino acid residues within the structures highlighted.

FIG. 12. Summary of biopanning candidates for the 5′ UTR Alpha-synuclein mRNAs. A table of wildtype (WT) TEXS3 and Nanobody sequences with enriched candidate sequences from each respective library biopanning. Variable amino acid residues within the WT TEXS3 and Nanobody Libraries are at positions: 18, 19, 22, 25, 26, 29, 102, 105, 106, 136, for the WT TEXS3 library; and positions: 28, 29, 30, 31, 32, 34, 35, 37, 56, 57, 58, 61, 103, 104, 105, 106, 107, 108, 109, 110, 111, 112, for the Nanobody library. Variable amino acid residues of each candidate at the respective variable positions are bolded.

FIG. 13. Kinetic analysis of SNCA candidates. Single-cycle kinetic analysis of FIG. 13A) Sumo-SNCA-B, FIG. 13B) Sumo-SNCA-C, FIG. 13C) Sumo-SNCA-E or FIG. 13D) Sumo binding to the 5′UTR of SNCA mRNA. All experiments were performed in triplicates and each representative sensogram depicts the blank- and reference-subtracted data with kinetic fit for the 1:1 interaction model shown as an overlaid thin black line. Each candidate was injected sequentially in order of increasing concentrations (312 nM, 625 nM, 1.25 UM, 2.5 M, 5 UM, 10 UM) without dissociation or regeneration between each sample concentration, followed by a final regeneration. Equilibrium constants (K_{D}) were determined by measuring the association rate constant (Kₐ) with a fixed 5% dissociation rate constant (K_{d}). FIG. 13E) Sensogram depicting Sumo-SNCA-C binding to the 5′UTR of SNCA mRNA. 10 UM of Sumo-SNCA-C was injected at a flow rate of 20 μl per minutes and its dissociation from SNCA mRNA was observed over a 90 minute period.

FIG. 14. Characterization of high affinity Proteimers targeting the 5′UTR of SNCA mRNA. Predicted binding of SNCA-B, SNCA-C, and SNCA-E to the hairpin of SNCA mRNA. FIG. 14A) The best scoring pose from an Alphafold3 prediction of SNCA-B bound to SNCA mRNA used for selection. Inset depicts a zoom-in of potential stabilizing interactions between designed SNCA-B residues Tyr41, His113, and Glu132 and the target mRNA. FIG. 14B) The best scoring pose from an Alphafold3 prediction of SNCA-C bound to SNCA mRNA used for selection. Inset depicts a zoom-in of potential stabilizing interactions between designed SNCA-C residues Asn18 and Arg19 and the target mRNA. FIG. 14C) The best scoring pose from an Alphafold3 prediction of SNCA-E bound to SNCA mRNA used for selection. Inset depicts a zoom-in of potential stabilizing interactions between designed SNCA-E residue Arg103 and the target mRNA.

FIG. 15. Summary of biopanning candidates for the 5′ UTR APP mRNAs. Alignment of the wild-type (WT) TEXS3 amino acid sequence with enriched variants identified through biopanning of a TEXS3 display library. Residues that were variable in the WT TEXS3 library are indicated in bold. Biopanning yielded two enriched clones, designated APPS3-5 and APPS3-11; and residues that differ in each enriched candidate are highlighted in bold.

FIG. 16. Characterization of high affinity Proteimers targeting the 5′UTR of APP mRNA. FIG. 16A) ELISA testing binding of APP Proteimers to 5′UTR of the APP mRNA. ELISA verified initial binding seen through biopanning in a concentration dependent manner from 37 nM to 1 μM. All experiments were performed in duplicates. FIG. 16B) Single Cycle kinetics of APP Proteimers binding to the 5′UTR APP mRNA. Kinetic fit was performed with a 1:1 binding model. Injections were performed in order of increasing concentration (62 nM, 125 nM, 250 nM, 500 nM, and 1 μM) without dissociation or regeneration between each injection. FIG. 16C) Among various previously generated Proteimers, only APPS3-11 showed specific binding to the 5′UTR of APP mRNA, while others targeting Tau mRNA or Fentanyl did not. FIG. 16D) In vitro RNA degradation assays using gel electrophoresis confirmed target-specific degradation by ProAPPS3-11. FIG. 16E) Predicted binding interaction between APPS3-5 and APP mRNA shows the 5′-AGA-3′ region unfolding into the binding pocket of the APPS3-5 Proteimer, where it is likely stabilized by several selected mutated amino acids. FIG. 16F) Predicted binding interaction between APPS3-11 and APP mRNA reveals the 5′-AGA-3′ region unfolding into the binding pocket of the APPS3-11 Proteimer, where it is likely stabilized by several mutated amino acids.

FIG. 17. Quantification of a western blotting of Proteimer ProAPPS3-11 inhibiting APP protein expression in SH-SY5Y neuroblastoma cells. FIG. 17A) The ProAPPS3-11 construct is composed of several functional domains: angiopep-2, PUM, APPS3 (5 or 11), an RNase domain, and an albumin-binding domain (ABD). FIG. 17B) Quantification of a western blotting of Proteimer ProAPPS3-11 inhibiting APP protein expression in SH-SY5Y neuroblastoma cells. Western blot analysis following 48 hours of treatment with ProAPPS3-11 at 1, 2, and 5 μg revealed a dose-dependent reduction, with FIG. 17C) APP protein levels decreasing to approximately 60% at the highest concentration. FIG. 17D) Cytotoxicity of treatment with ProAPPS3-11 at different concentrations measured by CyQUANT LDH cytotoxicity assay (ThermoFisher) represented as the average absorbance at 490 nm minus the reference absorbance at 680 nm for 3 samples for each treatment.

FIG. 18. Summary of biopanning candidates for the 5′ UTR tau mRNAs. Alignment of the wild-type (WT) TEXS3 amino acid sequence with enriched variants identified through biopanning of a TEXS3 display library. Residues that were variable in the WT TEXS3 library are indicated in bold. Biopanning yielded two enriched clones, designated TEX-T1 and TEX-T2; and residues that differ in each enriched candidate are highlighted in bold.

FIG. 19. Characterization of high affinity Proteimers targeting the 5′UTR of Tau mRNA. FIG. 19A) SPR analysis of Control (Sumo), TEX-T1, or Nano-T1 binding to the 5′UTR of Tau mRNA at 1 μM. FIG. 19B) Various target-specific Proteimers were evaluated for binding to the 5′UTR of APP mRNA. Among previously developed Proteimers targeting other bates (e.g., tau, SNCA mRNA,), only TEX-T1 specifically bound to the 5′UTR of tau mRNA. FIG. 19C) Sequence of the T7-tau template used for in vitro transcription and in vitro degradation (SEQ ID NO:172). T7 promoter sequence, (first 20 nucleotides); tau hairpin sequence used for biopanning, (next 30 nucleotides); 99 additional nucleotides (ntd) following the tau stem-loops; transcription start site, underlined; GG nucleotides added for enhance transcription, bold FIG. 19D) Cartoon representation of the Proteimer-RNAse fusion protein bound to its target mRNA. FIG. 19E) Tau mRNA degradation assay. FIG. 19F) LDH release from different concentrations of ProTEX-T1 after 48 h. FIG. 19G) Predicted model of TEX-T1 binding to tau mRNA. The best scoring pose from an Alphafold3 prediction of TEX-T1 bound to the Tau mRNA segment. Inset depicts potential stabilizing interactions between TEX-T1 and its target mRNA. FIG. 19H) Predicted binding of Nano-T1 to the tau mRNA. The best scoring pose from an Alphafold3 prediction of Nano-T1 bound to tau mRNA used for selection. Inset depicts a zoom-in of potential stabilizing interactions between designed Nano-T1 residues Arg103 and the target mRNA.

FIG. 20. Summary of Biopanning-Derived TEXS3 Variants for CD19. Alignment of the wild-type (WT) TEXS3 amino acid sequence with enriched variants obtained through biopanning of the TEXS3 display library. Positions that were variable in the original library are indicated in bold, while residues differing in each enriched clone are highlighted in bold. Biopanning against CD19 yielded two dominant variants, designated TEX-CD19_6 and TEX-CD19_8.

FIG. 21. Binding activity of CD19-specific aptamer fusion proteins assessed by ELISA and SPR. FIG. 21A) ELISA binding curves showing concentration-dependent recognition of CD19 by CD19-6 MBP and CD19-8 MBP, but not by the CD3 control aptamer or blank control. Data demonstrate specific binding of engineered CD19 constructs. FIG. 21B) Surface plasmon resonance (SPR) sensorgram of MBP-C19_8, showing strong and specific binding kinetics to CD19. FIG. 21C) SPR response of MBP control, indicating no measurable interaction with CD19.

FIG. 22. Structural models of TEXS3 variants binding to CD19. FIG. 22A) Predicted interaction model of TEX-CD19_6) binding to CD19. Mutated residues enhance binding affinity by forming close-range interactions (<3.5 Å) concentrated around two helices at the pocket entrance. Aromatic residues from CD19 and a potential hydrogen bond between Arg26 and Thr176 contribute to stabilization. FIG. 22B) Predicted binding of TEX-CD19_8 to CD19. Three major interaction regions are identified: (1) aromatic stabilization around Asn46, (2) helix 2 interaction with Arg231 of CD19, and (3) a diverse set of van der Waals and hydrogen bonding interactions at the lower binding interface.

FIG. 23. FIG. 23A) Diagram illustrating Antibody-dependent cellular cytotoxicity (ADCC) activity of CD19-Proteimer. FIG. 23B) Target cell lysis was measured in the presence of NK effector cells at increasing effector-to-target (E:T) ratios. CD19-Proteimer (corresponding to TEX-CD19_8-Fc; SEQ ID NO:178) induced robust ADCC comparable to the clinical anti-CD20 antibody obinutuzumab, while IgG isotype and no effector controls showed minimal activity. FIG. 23C) Expression of hCD19 and hCD20 in OSU-CLL cells. Flow cytometry analysis of OSU-CLL cells demonstrating surface expression of hCD19 and hCD20. These cells were used as target cells in subsequent ADCC assays.

FIG. 24. Possible mechanisms of CD19-Proteimer-mediated activity. CD19-Proteimer binds CD19 on B cells and triggers multiple immune effector pathways, including antibody-dependent cellular cytotoxicity (ADCC) by natural killer cells, antibody-dependent cellular phagocytosis (ADCP) by macrophages, and complement-dependent cytotoxicity (CDC). Additionally, CD19-Proteimer may induce direct cell death (DCD) through lipid raft-associated signaling.

FIG. 25. FIG. 25A-FIG. 25D. Summary of Biopanning-Derived TEXS3 Variants for various target (SNCA, APP, Tau, Ferritin, Fentanyl, Cortisol, CD19, CD3, CD33, CD37, Hur, CD20, CD22, Bcl2, Her2, APP peptide, TED Variants for various target (TAAR1, TARR1-ECL2, TARR1-ECL1, CD37, CD3, CD20, CD22). Alignment of the wild-type (WT) TEXS3 amino acid sequence with enriched variants obtained through biopanning of the TEXS3 display library.

FIG. 26. Summary of Biopanning-Derived Nanobody Variants for various target. Alignment of the wild-type (WT) Nanobody amino acid sequence with enriched variants obtained through biopanning of the Nanobody display library.

FIG. 27. Summary of Biopanning-Derived YTH Variants for various target. Alignment of the wild-type (WT) YTH amino acid sequence with enriched variants obtained through biopanning of the YTH display library.

FIG. 28. Summary of Biopanning-Derived ACA2 Variants for various target (5′ UTR mRNA of cMyc-A323M, ARID5A). Alignment of the wild-type (WT) ACA2 amino acid sequence with enriched variants obtained through biopanning of the ACA2 display library.

FIG. 29. FIG. 29A) Schematic representation of protocol for identifying spike-RBD binding aptamers from collection of rational engineered Gryl-like variants and positive recombinants selected from phage display using ELISA assay. FIG. 29B) ELISA assay results that prove Gryl-like variants bind to spike-RBD with high affinity.

FIG. 30. FIG. 30A) Gene and amino acid sequences of Gyrl-like variants CTR Y106W (SEQ ID NO:8) and SAV HS (SEQ ID NO:9) FIG. 30B) Alignment of CTR Y106W to wildtype CTR107. FIG. 30C) Alignment of SAV HS to wildtype SAV2435.

FIG. 31. Structural models comparing Gyrl-like variant SAV HS to wildtype SAV2435, respectively. Binding site residues are represented as black or white for SAV HS and SAV2345 WT, respectively.

FIG. 32. Comparison of spike-binding by Gryl-like aptamers to human monoclonal SARS-COV-2 antibody and human ACE2 receptor. Protocol is same as described in FIG. 1A.

FIG. 33. FIG. 33A) Schematic representation of the protocol used to study inhibition of the interactions between spike-RBD and ACE2 receptor or spike-RBD and SARS-COV-2 antibody by Gyrl-like aptamers FIG. 33B) Inhibition of spike-RBD-ACE2 binding by Gyrl-like aptamers. FIG. 33C) Inhibition of spike-RBD-Antibody binding by Gyrl-like aptamers.

FIG. 34. FIG. 34A) Schematic representation of the protocol used to study inhibition of the interactions between the spike-RBD and ACE2 receptor after coating ELISA plates with spike-RBD or spike trimer. FIG. 34B) Inhibition of Spike-RBD-ACE2 binding by Gyrl-like aptamers after coating plate with spike-RBD. FIG. 34C) Inhibition of Spike-RBD-ACE2 binding by Gyrl-like aptamers after coating plate with spike trimer.

FIG. 35. Dose-response inhibition of the interactions between spike-RBD and ACE2 receptor by Gyrl-like aptamers.

FIG. 36. FIG. 36A) Comparison of structural models of engineering Proteimer SAV-HS to wild-type SAV2435. FIG. 36B) Competitive inhibition of SARS-COV-2 cell entry mechanism by Proteimer aptamer.

FIG. 37. FIG. 37A) Comparisons of interactions at the SAV WT-SARS-COV-2 RBD and SAV-HS-SARS-COV-2 RBD, SAV-HS-SARS-COV-2 RBD variant using AlphaFold2 prediction of protein-protein interaction of Proteimer aptamer. FIG. 37B) A closer examination of the protein interface between SAV-HS and SARS-COV-2 RBD reveals a detailed view of their interaction. 20 amino acids were predicted and highlighted for atomic distance 3.5 A to RBD by the HDOCK molecular docking of protein-protein interaction. FIG. 37C) Inhibition of Spike-RBD-ACE2 interactions from SARS-COV-2 variants. FIG. 37D) Sequence alignment of the SARS-COV-2 (SEQ ID NO:173) and SARS-COV (SEQ ID NO:174) RBDs. FIG. 37E) Comparing the epitope mapping of SAV-HS with neutralizing monoclonal antibodies (mAbs) and ACE2.

### DETAILED DESCRIPTION OF THE INVENTION

Provided herein are protein aptamers (Proteimers) that can be selectively designed to target the repertoire of target molecules to include RNA, DNA, protein molecules, posttranslational modification groups, peptides, and prosthetic. Protein molecules with surface-exposed epitopes or post-translational modification are attractive targets for generating specific protein aptamers (Proteimers) through in vitro evolution methods. Protein screening and design have become central to therapeutic discovery, utilizing the vast diversity of natural protein structures, functions, and interactions. The invention Proteimer designs harness this diversity, permitting the high-throughput development of high-affinity binders for small molecules, proteins, and nucleic acids.

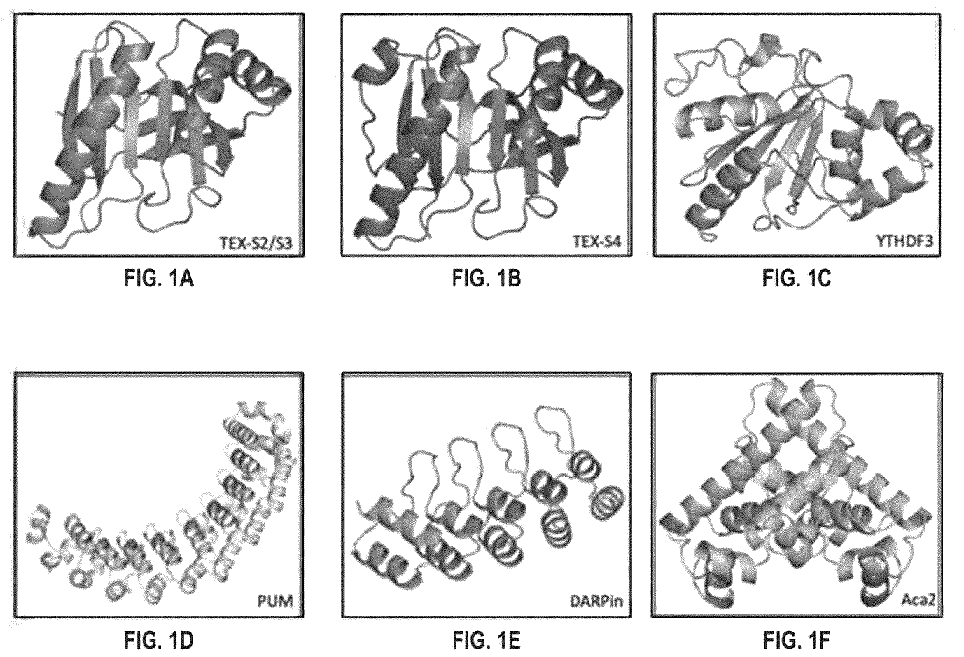

The invention Proteimer libraries were intially screened in silico using tools such as AlphaFold3, and the like, to evaluate stability and guide design. These libraries enhance native binding modes of scaffold proteins by introducing targeted mutations at key amino acid positions to modulate binding specificity. Six unique protein scaffolds—TEX-S2/S3 (Gyrl-like), TEX-S4 (Gyrl-like), YTHDF3 (RNA binding domain), PUM (RNA binding domain), DARPin, and Aca2 (RNA binding domain)—have been developed to provide specificity and affinity comparable to antibodies and antibody mimetics across a broad spectrum of therapeutic targets. (FIG. 1).

Gyrl-like proteins (TEX264, CTR107, LIN2189, SAV2435) contain diverse binding pockets that bind to a wide range of organic structures; and randomization of Gryl-like protein binding sites according the present invention has produced novel and expansive binding surfaces that can interact with diverse peptides or post-translational modification groups on the surface of proteins. Previous related inventions demonstrated methods for creating aptamers from select Gyrl-like proteins CTR107, LIN2189, SAV2435 (U.S. Pat. No. 12,104,201). These libraries, which are comprised of variants defined by randomization of specific binding site residues that are known to interact with small molecules, serve as a collection of aptamers that can bind with high affinity to any target molecule. Through phage display selection methods employed herein, unique aptamers have been isolated and identified through gene sequencing. In accordance with the present invention, Proteimers that function as drug binders, fluorescence activators or quencher and enzymes, have been isolated. These functional protein aptamers are referred to herein as “Proteimers” or “Proteimer”.

Accordingly, provided herein is a non-native variant proteimer, relative to wild-type TEX264, comprising any combination of one up to all 8 of variant amino acid residues, wherein the variant amino acid residues correspond to residues 18, 19, 25, 26, 29, 102, 105 and 106 of TEX-S2 (SEQ ID NO:2). In one embodiment, the number of variant amino acid residues is selected from the group consisting of: 1, 2, 3, 4, 5, 6, 7, and 8.

Also provided herein, is a non-native variant proteimer, relative to wild-type TEX264, comprising any combination of one up to all 12 of variant amino acid residues, wherein the variant amino acid residues correspond to residues 18, 19, 22, 25, 26, 29, 40, 46, 102, 105, 106, and 136 of TEX-S3 (SEQ ID NO:4). In a particular embodiment, the number of variant amino acid residues is selected from the group consisting of: 1, 2, 3, 4, 5, 6, 7, 8, 9, 10, 11 and 12. In another embodiment, the proteimer is selected from the group consisting of SEQ ID NOs: 16 (SNCA-B), 17 (SNCA-C), 21 (APPS3-5), 22 (APPS3-11), 24 (TEX-T1), 25 (TEX-T2), 27 (TEX-CD19-6), 28 (TEX CD19-8), and 30-110. In another embodiment, the proteimer is selected from the group consisting of SEQ ID NOs: 16 (SNCA-B), 17 (SNCA-C), 21 (APPS3-5), 22 (APPS3-11), 24 (TEX-T1), 25 (TEX-T2), 27 (TEX-CD19-6), 28 (TEX CD19-8).

Also provided is a non-native variant proteimer, relative to wild-type TEX264, comprising any combination of one up to all 12 of variant amino acid residues, wherein the variant amino acid residues correspond to residues 31, 32, 35, 38, 39, 42, 53, 59, 115, 118, 119, and 149 of TEX-S4 (SEQ ID NO:6). In a particular embodiment, the number of variant amino acid residues is selected from the group consisting of: 1, 2, 3, 4, 5, 6, 7, 8, 9, 10, 11 and 12.

Also provided is a non-native variant proteimer, relative to wild-type YTHDF3, comprising any combination of one up to all 11 of variant amino acid residues, wherein the variant amino acid residues correspond to residues 40, 54, 55, 57, 84, 108, 113, 129, 147, 149, and 150 of YTHDF3 (SEQ ID NO:8). In one embodiment, the number of variant amino acid residues is selected from the group consisting of: 1, 2, 3, 4, 5, 6, 7, 8, 9, 10, and 11. In a particular embodiment, the proteimer is selected from the group consisting of SED ID NOs: 124-138.

Also provided is a non-native variant proteimer, relative to wild-type PUM, comprising any combination of one up to all 24 of variant amino acid residues, wherein the variant amino acid residues correspond to residues 37, 38, 41, 73, 74, 77, 109, 110, 113, 145, 146, 149, 181, 182, 185, 217, 218, 221, 253, 254, 257, 296, 297, and 300 of PUM (SEQ ID NO:10). In a particular embodiment, the number of variant amino acid residues is selected from the group consisting of: 1, 2, 3, 4, 5, 6, 7, 8, 9, 10, 11, 12, 13, 14, 15, 16, 17, 18, 19, 20, 21, 22, 23, and 24.

Also provided is a non-native variant proteimer, relative to wild-type DARPin, comprising any combination of one up to all 21 of variant amino acid residues, wherein the variant amino acid residues correspond to residues 32, 34, 35, 37, 45, 46, 58, 65, 67, 68, 70, 78, 79, 91, 98, 100, 101, 103, 111, 112, and 124 of DARPin (SEQ ID NO:12). In one embodiment, the number of variant amino acid residues is selected from the group consisting of: 1, 2, 3, 4, 5, 6, 7, 8, 9, 10, 11, 12, 13, 14, 15, 16, 17, 18, 19, 20, and 21.

Also provided is a non-native variant proteimer, relative to wild-type Aca2, comprising any combination of one up to all 14 of variant amino acid residues, wherein the variant amino acid residues correspond to residues 28, 30, 31, 33, 34, 39, 45, 149, 151, 152, 154, 155, 160, and 166 of Aca2 (SEQ ID NO:14). In one embodiment, the number of variant amino acid residues is selected from the group consisting of: 1, 2, 3, 4, 5, 6, 7, 8, 9, 10, 11, 12, 13, and 14. In a particular embodiment, the proteimer is selected from the group consisting of SED ID NOs: 141-148.

Also provided is a non-native variant proteimer, relative to a wild-type nanobody, comprising any combination of one up to all 22 of variant amino acid residues, wherein the variant amino acid residues correspond to residues 28, 29, 30, 31, 32, 34, 35, 37, 56, 57, 58, 61, 103, 104, 105, 106, 107, 108, 109, 110, 111, and 112 of the nanobody (SEQ ID NO:18). In one embodiment, the number of variant amino acid residues is selected from the group consisting of: 1, 2, 3, 4, 5, 6, 7, 8, 9, 10, 11, 12, 13, 14, 15, 16, 17, 18, 19, 20, 21, and 22. In a particular embodiment, the proteimer is selected from the group consisting of SED ID NOs: 112-121.

Also provided is a chimeric proteimer construct comprising,
a proteimer moiety set forth heron; and
a recombinant polypeptide moiety. In particular embodiments, the polypeptide moiety is selected from one or more of a proteolytic degradation moiety, an Fc-region moiety and/or one or more other proteimer moieties set forth herein. Accordingly, it is contemplated herein that an invention chimeric proteimer construct can comprise two or more invention proteimer moieties that bind to different proteins, such as different cell surface cancer targets and/or different RNA targets, such as 5′ UTRs of various mRNAs.

In particular embodiments, the proteolytic degradation moiety is selected from proteases and/or nucleases. In one embodiment, the nuclease moiety is an RNAse moiety selected from one or more of: RNase A, PIN, SMG6 PIN, RENT1/UPF1 PIN, viral PIN domains, RNase MRP, RelE toxin PIN, Cas13, Onconase, RNAse 2, RNAse 3, RNAse 5, Barnase, Binase and/or pinard. In particular embodiments, the chimeric proteimer binds and cleaves a specific RNA target. In other embodiments, the RNA target comprises an Internal Ribosomal Entry Site (IRES) and/or an Iron-Responsive Element (IRE). In other embodiments, the RNA target is a 5′ UTR region of an mRNA transcript coding for a protein selected from: c-myc, APP, tau, SNCA (Alpha-synuclein), ARID5A, A323M, Ferritin, and C-jun.

In yet another embodiment, the invention chimeric proteimer construct further comprises one or more of a pumilo (PUM) domain, a blood-brain barrier transcytosis domain, and a half-life extending domain. In one embodiment, the pumilo (PUM) domain can be one of the invention non-native variant PUM proteimers provided herein. In another embodiment, the blood-brain barrier transcytosis domain can be selected from Angiopep-2 peptide, HIV-1 Tat peptide, Rabies Virus Glycoprotein (RVG29), RDP-shuttle peptide, and/or Melanotransferrin-derived peptides. In yet another embodiment, the half-life extending domain can be selected from a transferrin receptor targeting protein domain, an FcBP domain, PEGylation, XTEN, PASylation and/or an albumin binding domain.

In a particular embodiment, the pumilo domain is amino acids 27-376 of SEQ ID NO: 176; the blood-brain barrier transcytosis domain is Angiopep-2 peptide; and the half-life extending domain is an albumin binding domain. As set forth in SEQ ID NOs: 176, 177 and 180, the various domains of the invention chimeric proteimer construct can be separate by a peptide linker sequence, such -Gₓ-S-, and the like. In particular embodiments, the proteimer moiety is selected from the group consisting of SEQ ID NOS: 16, 17, 19, 21, 22, 24, 25, 30-38, 112-115, 124-138, and 141-148. In other embodiments, the protease moiety can be obtained from one or more of: TRIM21, VHL, CRBN, MDM2, RNF4, FBW7, CHIP, FBXW7, Subtilisin, Trypsin, TEV protease, Granzyme B, Caspases, Papain, Cathepsins, Calpain, MMPs, ADAMs, HIV protease, and/or Cathepsin D. In one embodiment, the chimeric proteimer binds and cleaves a specific protein target.

In another embodiment, the polypeptide moiety is an Fc-region moiety and the proteimer moiety is selected from the group consisting of SEQ ID NOs: 27, 28, 40-110, and 116-121. In a particular embodiment, the chimeric proteimer construct is selected from: TEX-CD19_8: Fc-region (SEQ ID NO: 178); and/or TEX-CD19_6: Fc-region (SEQ ID NO: 179).

Also provided is a chimeric multivalent proteimer construct comprising, a first proteimer moiety selected from the invention non-native variant proteimers provided herein; and
a second proteimer moiety selected from the invention non-native variant proteimers provided herein. Also contemplated herein is more than two, e.g., 3, 4, 5, 6, 7, 8, 9, 10 or more, invention proteimer moieties within the a single chimeric multivalent proteimer construct. In another embodiment, the multivalent proteimer further comprises a half-life extending domain. In particular embodiments, the half-life extending domain can be selected from a transferrin receptor targeting protein domain, an FcRn-binding peptide domain, Fc-region, PEGylation, XTEN, PASylation and/or an albumin binding domain. In a particular embodiment, the half-life extending domain is an Fc-region. In another embodiment, the first and second proteimer moiety binds to a protein selected from the group consisting of: CD19, CD3, CD33, CD37, Hur, CD20, CD22, Bcl2, Her2, CD37. In yet other embodiments, the first and second proteimer moiety are selected from any combination of 2 or more of SEQ ID NOs: 27, 28, 47-62, 73-110, and 116-121. In a particular embodiment, the first and second proteimer moiety are selected from any combination of 2 or more of SEQ ID NOs: 27, 28, 47, 48, 55-57, 93-103 and 119, wherein the first protiemer moiety binds to CD19 and the second proteimer moiety binds to CD20.

Also provided herein, is a method of treating cancer, comprising administering to a patient in need thereof, an invention chimeric proteimer construct, wherein the proteimer moiety is selected from one or more of SEQ ID NOs: 27, 28, 47-62, 73-110, 116-121, 131-138, and 141-148. In certain embodiments, the proteimer moiety binds to a protein selected from the group consisting of: CD19, CD3, CD33, CD37, Hur, CD20, CD22, Bcl2, Her2, CD37. In a particular embodiment, the proteimer moiety binds to CD19. In another embodiment, the proteimer moiety is SEQ ID NO: 27 and/or SEQ ID NO: 28. In yet another embodiment, the chimeric proteimer construct is SEQ ID NO: 178 (CD19_8-Fc) and/or SEQ ID NO:179 (CD19_6-Fc). In one embodiment, the proteimer moiety binds to a 5′ UTR mRNA target coding for a protein selected from the group consisting of: cMyc-A323M and ARID5A. In other embodiments, the proteimer moiety is selected from one or more of SEQ ID NOs: 131-138 and 141-148.

Also provided herein is a method of treating Alzheimer's disease, comprising administering to a patient in need thereof, a chimeric proteimer construct of claims 22-32, wherein the proteimer moiety is selected from one or more of SEQ ID NOs: 21, 22, 24, 25, 32-38, 63, 64, 113-115, and 124-130. In particular embodiments, the proteimer moiety binds to a 5′ UTR mRNA target coding for a protein selected from the group consisting of: APP, Tau, Ferritin, and Tau60. In another embodiment, the proteimer moiety binds to a 5′ UTR mRNA target coding for APP. In other embodiments, the proteimer moiety is SEQ ID NO: 21 and/or SEQ ID NO:22. In yet other embodiments, the chimeric proteimer construct is SEQ ID NO: 176 (ProAPPS3-5) and/or SEQ ID NO: 177 (ProAPPS3-11). In a particular embodiment, the proteimer moiety binds to a 5′ UTR mRNA target coding for Tau. In another embodiment, the proteimer moiety is SEQ ID NO: 24. In yet another embodiment, the chimeric proteimer construct is SEQ ID NO: 180 (ProTEX-T1).

Also provided herein is a method of treating Parkinson's disease, comprising administering to a patient in need thereof, a chimeric proteimer construct of claims 22-32, wherein the proteimer moiety is selected from one or more of SEQ ID NOs: 16, 17, 19, 30, 31, and 112. In certain embodiments, the proteimer moiety binds to a 5′ UTR mRNA target coding for alpha-Synuclein.

Additionally, several therapeutic aptamer candidates for SARS-COV-2 spike protein were identified as a proof-of-principle for protein recognition by Gyrl-like aptamers. A functional assay was developed for testing the function of Gyrl-like aptamers that bind to SARS-COV-2 spike protein. Using this assay, two unique aptamers SAV HS and CTR Y106W were identified that bind to spike-RBD comparably to or better than control human ACE2 receptor. Further modification of assay to compare unique Gyrl-like Proteimers to human SARS-COV-2 monoclonal neutralizing antibody proved that the affinity of the invention Proteimers spike-RBD is high and within the desired range for therapeutic effects. A further modification of the ELISA binding assay to screen for inhibitory effects of Gyrl-like Proteimers on the complex formed between spike-RBD and ACE2 receptor is provided. This inhibitory assay validates the potential therapeutic effects of Gyrl-like aptamers for disrupting Spike-RBD and prevent host entry. The assay protocol is versatile and coating can be conducted with any protein as bait, including the functional full-length spike protein in a trimer complex. Inhibition of spike trimer is nearly identical to spike-RBD highlighting specificity of inhibition site and inhibition of the physiological complex involved in host cell entry. Dose-dependent inhibition shows that SAV HS can produce maximal inhibition at concentrations as low as 2 nM which is similar to neutralizing monoclonal SARS-COV-2 antibody. Herein, the presented examples below provide a proof-of-principle for the development of SARS-COV-2 therapeutic Proteimers from the Gyrl-like protein family. Using the methodology provided herein, Proteimers that bind to protein targets with high affinity can be rapidly isolated. The invention technology is important for creating rapid therapeutics that can treat new strains of SARS-COV-2 or novel pandemic coronavirus. Furthermore, the invention technology represents a prototype for developing versatile Proteimer biotherapeutics that can function similar to engineered monoclonal antibodies.

### EXAMPLES

The following experimental data details the extension of a new protein scaffold library, introduces a new target, and outlines the methods and protocols for isolating Proteimer (protein aptamers) specific to target proteins.

### Materials and Methods

### Phage Display

NNK library designs described in related patent U.S. Pat. No. 12,104,201 are cloned into T7 bacteriophage using the T7Select10-3 cloning kit and phage display lysates prepared according to protocol (Millipore Sigma, Burlington, MA). 1 μM Biotinylated Spike-RBD protein (Acro Biosystems Newark, DE) is immobilized to 5 μl polystyrene beads conjugated with streptavidin by incubating for 30 mins at room temperature. Beads are then washed by 5-7 rounds of washing and centrifugation at 10,000 RPM with 150 μl wash buffer (1×PBS containing 0.5% TWEEN20). Phage libraries are then added to Spike-RBD coated beads and incubated for 30 mins, followed by 5-7 rounds of washing with wash buffer and centrifugation. Bound phages are eluted from beads using 1×PBS contain 1 mM biotin and centrifuged to remove beads. The supernatant containing eluted phages is used to infect E. coli BLT5403 cells according to T7Select10-3 cloning kit protocol. Lysate from previous round of biopanning is used to conduct each successive round of selection. The above protocol is repeated for 6-8 rounds and plaque assay as described in T7Select10-3 cloning kit is used to identify positive recombinants. Recombinants are sequenced to confirm identity then cloned, expressed and purified according to U.S. Pat. No. 12,104,201 for further functional testing.

### ELISA Assay for Detecting Spike-RBD Binding

A rapid ELISA assay was developed that requires no overnight coating. The first step involves coating COSTAR high bind ELISA plates (Corning Inc. Corning, NY) with 100 μl of 0.5 μg/ml of aptamer, human SARS-COV-2 monoclonal antibody (Acro Biosystems Newark, DE), human ACE2 (Acro Biosystems Newark, DE) or negative control FITC antibody (Thermo Fisher Scientific, Waltham, MA). Plates are incubated for 2 hrs at 37° C. then washed three times with ELISA wash buffer (1×PBS containing 0.05% TWEEN20), followed by blocking with 300 μl blocking buffer (1×PBS with 2% BSA) for 2 hrs. After blocking and washing away excess blocking buffer 100 μl of 25 nM of biotinylated SARS-COV-2 Spike-RBD protein (Acro Biosystems Newark, DE) was added to wells and plate incubated for 1 hr at 37° C., followed by washing three times with wash buffer to remove unbound protein. Next, 100 μl of 0.1 μg/ml Streptavidin-HRP (Acro Biosystems Newark, DE) was added and plate incubated for 1 hr at 37° C. A TMB working solution was added after washing excess Streptavidin-HRP away and incubating plate at 37° C. for 1 hr. The absorbance of plate is measured on a TECAN infinite M200 pro plate reader at 450 nm with reference wavelength at 600 nm.

Alternatively, Biotinylated spike-RBD can be replaced with spike-RBD construct contain a mouse Fc tag and goat-anti-mouse antibody-HRP conjugate used for signal detection. In some aspects spike-RBD-Fc can produce lower nonspecific binding or higher Signal to noise and its recommended that both methods be used in conjunction to confirm results. A schematic description of the protocol is described in FIG. 1A.

### ELISA Assay for Detecting Inhibition of Spike Protein by Gyrl-Like Aptamers

For inhibition experiments the above protocol is modified to coat plates with 100 μl of 0.5 μg/ml ACE2 receptor, human SARS-COV-2 monoclonal antibody, spike protein-RBD or stabilized full length spike protein trimer (Bei resources, Manassas, VA). For experiments where coating is done with ACE2 or SARS-COV2 antibody 50 μl of 25 nM biotinylated spike-RBD is used in binding step and incubated 37° C. for 30 mins. Next 50 μl of 100 nM aptamer or control protein is added and allowed to incubate at 37° C. for 30 mins. Control wells without aptamer or control protein receives 50 μl of wash buffer to compensate for volume. Signal detection is carried out as described above. For experiments where coating is done with spike-RBD or spike trimer, 50 μl of 25 nM biotinylated ACE-2 (Acro Biosystems Newark, DE) is used. Signal detection is carried out with Streptavidin-HRP as described above.

### Example 1: TEX-S2 Proteimer Library

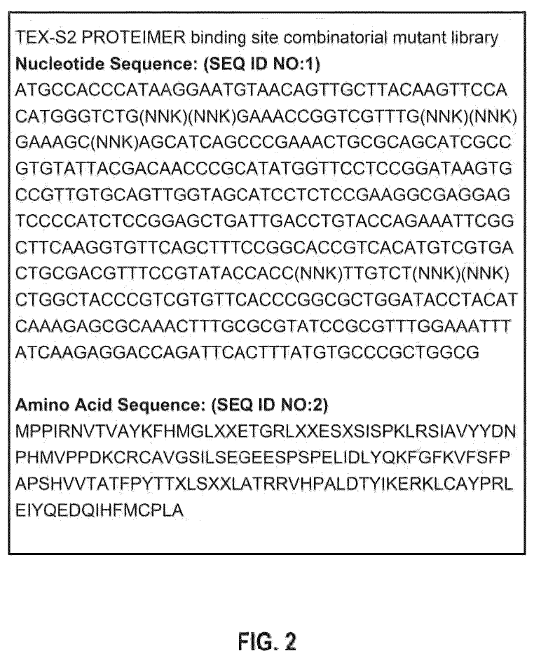

The TEX-S2 Proteimer library is based on the Gyrl-like domain of the human protein TEX264. Wild type TEX264 is a multi-domain regulatory protein which promotes the degradation of ER proteins during starvation by remodeling ER subdomains and functions in TOP1cc DNA repair. This scaffold was chosen due to its close homology to bacterial Gyrl-like proteins, such as CTR107 and LIN2189 (FIG. 1A). A total of eight amino acid sites, all in and around the putative ligand binding pocket, were selected as variable positions for the Proteimer library and NNK degenerate codons were utilized to cover the full breadth of amino acid diversity (FIG. 2). The library's diversity has been quantified at 2.56×10¹⁰.

### Example 2: TEX-S3 Proteimer Library

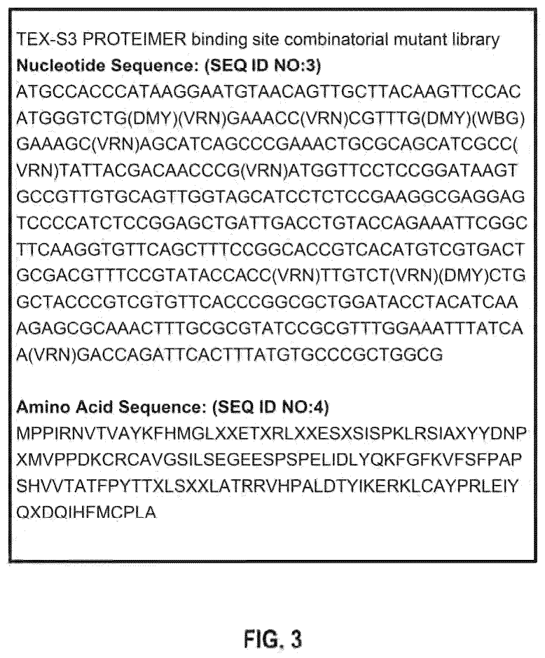

The TEX-S3 Proteimer library is similar to the TEX-S2 library described above. However, for TEX-S3, an additional four amino acid positions in proximity to the putative binding pocket and likely relevant to ligand binding were selected as variable sites (FIG. 1A). In addition, more restrictive degenerate codons were selected to limit nonproductive amino acid inclusion and stop codons while maintaining a suitable level of diversity (FIG. 3). The library's diversity has been quantified at 5.58×10¹⁰.

### Example 3: TEX-S4 Proteimer Library

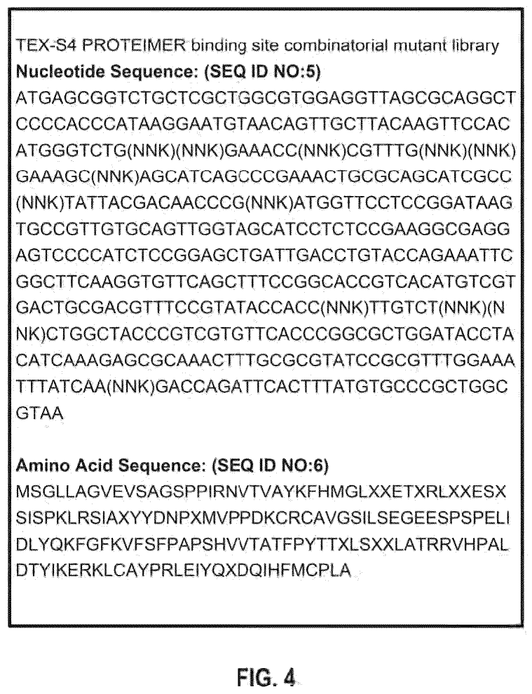

The TEX-S4 Proteimer library is an extension of TEX-S3 design (FIG. 1B). For this library, the same 12 variable amino acid positions were maintained but utilized NNK degenerate codons to cover the entire amino acid diversity available (FIG. 4). Additionally, an N-terminal β-strand segment from the wildtype sequence was included in this construct based on structural predictions showing that this small segment may provide favorable protein stability. The library's diversity has been quantified at 4.10×10¹⁵.

### Example 4: YTHDF3 Proteimer Library

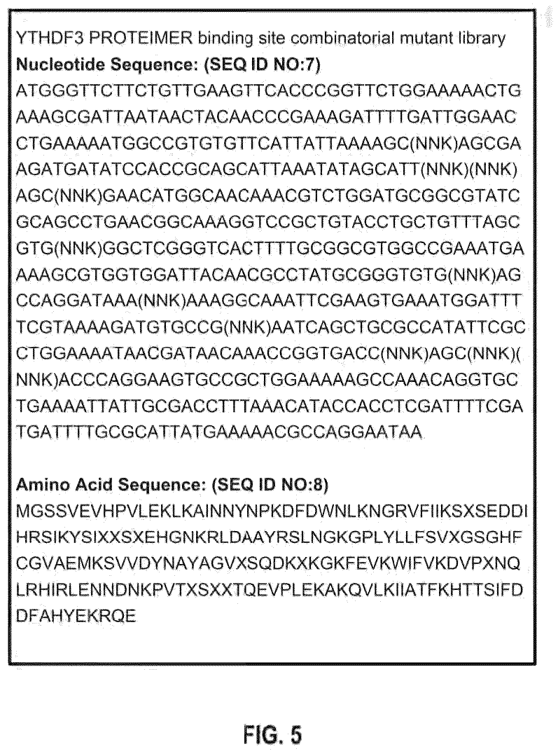

The YTHDF3 Proteimer library is based on the well-characterized YTH protein domain, which serves as the design scaffold for creating targeted Proteimers. Wild-type YTHDF3 is a human RNA binding protein which recognizes the N6-methyladenosine (m6A) modification of mRNA in cells, acting as a nucleic acid reader and regulatory protein (Li et al., 2020). This scaffold provides a known basis for nucleic acid recognition and allows tailoring of the outer binding surface and binding pocket to new ligands (FIG. 1C). To this end, the YTHDF3 library includes variable amino acids at 11 positions all focused on modifying the native RNA binding residues (FIG. 5). These positions are varied using NNK degenerate codons to encompass all amino acid diversity at each position. The library's diversity has been quantified at 2.05×10¹⁴.

### Example 5: PUM Proteimer Library

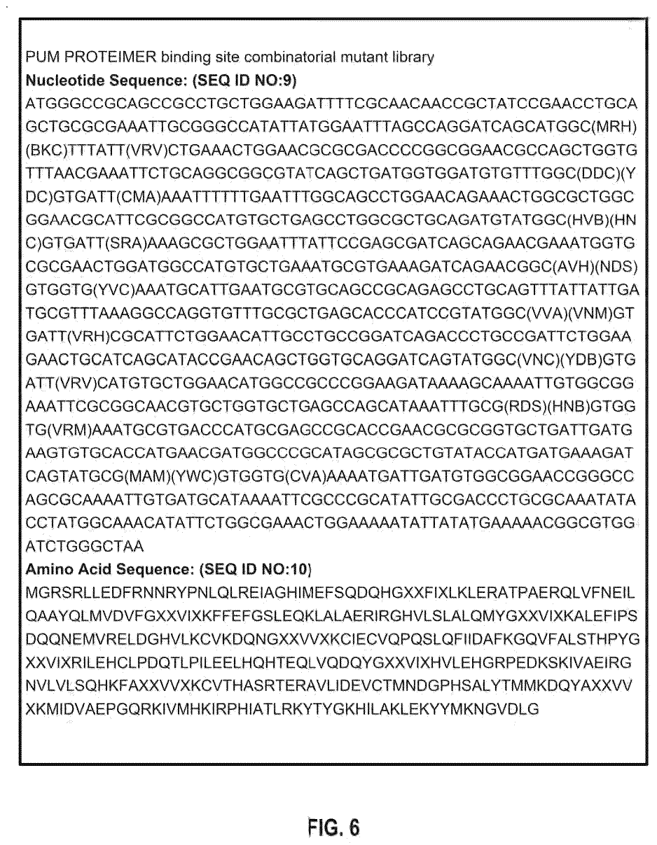

The PUM Proteimer library described here is a designed variant of the human Pumilio 1 PUF protein domain. This domain is a well characterized RNA binding motif which strictly recognizes single-stranded RNA with sequence specificity. The eight tandem repeating units of the PUF domain each contain three amino acids which confer sequence specificity for a particular nucleic acid base (FIG. 1D). Previous work has described and expanded the codex of amino acids that confer this specificity. Based on the identified variability at each position of each repeat (24 amino acid positions in total), degenerate codons were selected which capture the sequence diversity and avoid stop codons to provide a varied library capable of identifying highly specific single-stranded RNA binders (FIG. 6). The library's diversity has been quantified at 6.71×10²⁰.

### Example 6: DARPin Proteimer Library

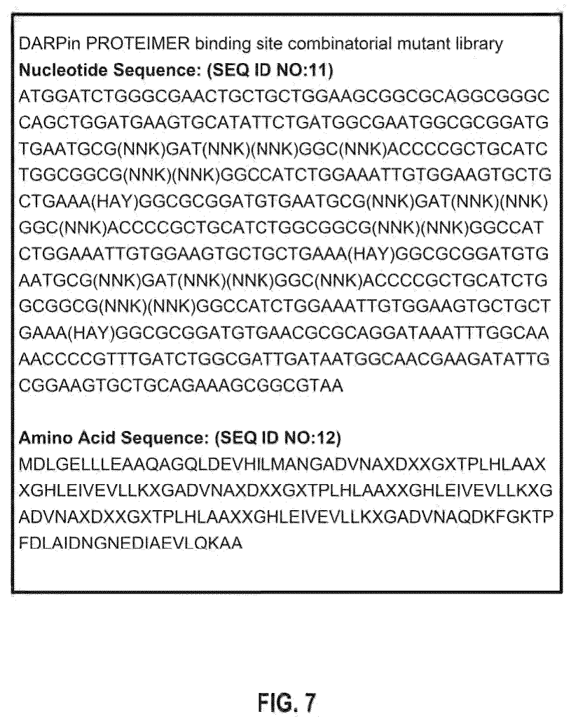

The DARPin Proteimer library described here uses the designed ankyrin repeat protein scaffold with various modifications. The DARPin structure consists of three internal tandem repeating alpha-helical domains flanked by N- and C-terminal repeats that function as stabilizing regions (FIG. 1E). DARPin proteins have been utilized widely as antibody-like protein-protein binders and are being explored as therapeutics molecules. The invention library design incorporates previous DARPins in which each of the three repeats in this design contains seven variable amino acid positions; six NNK degenerate codon sites on the canonical binding surface and one less diverse site on the anterior region of the protein (FIG. 7). In addition, the invention library also includes a number of stabilizing mutations including in the C-Cap region and N-cap region as well as protease resistance mutations throughout. The resulting DARPin library provides a diverse range of binding surface possibilities on a highly stabilized scaffold. The library's diversity has been quantified at 2.36×10²⁴.

### Example 7: Aca2 Proteimer Library

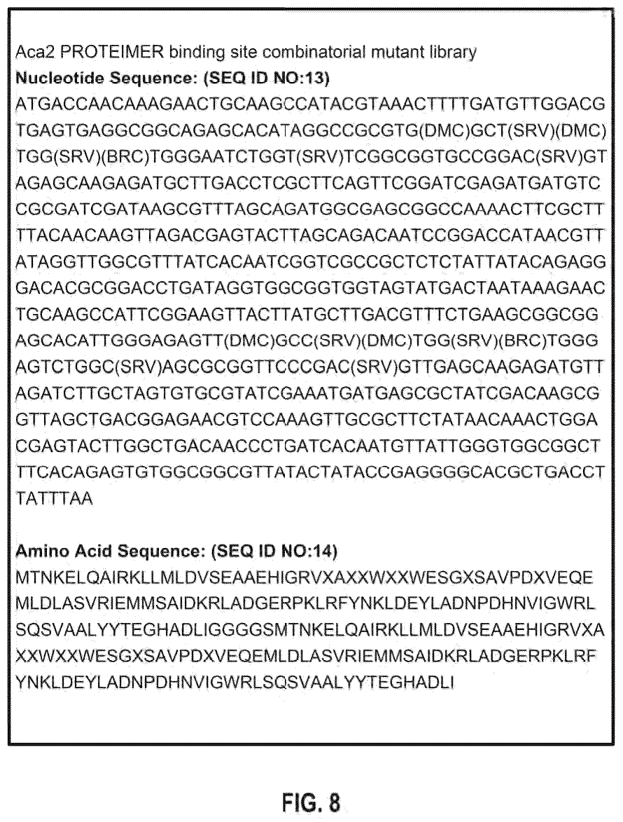

The Aca2 Proteimer library is based on the anti-CRISPR-associated (Aca) protein from the Pectobacterium phage ZF40. Aca2 is a helix-turn-helix (HTH) protein which functions as a dual RNA/DNA binding protein that can simultaneously regulate cellular transcription and translation. Because Aca2 functions as a homodimer in its native state, a construct was designed which fuses two of the subunits through a short protein linker, yielding a compact protein which recapitulates the ordered region of the Aca2 homodimer (FIG. 1E). Given its known RNA hairpin binding capabilities, Aca2 is a promising candidate for RNA binder library design. To this end, the amino acids positions which contact the RNA hairpins in the native structure were varied. The degenerate codon set utilized here is more restrictive to encompass the natural sequence diversity observed at these positions, which is largely polar residues, and to avoid unproductive mutations or stops (FIG. 8). The invention Aca2 Proteimer library is a focused design which is able to confer a combination of sequence specificity through amino acid-nucleotide contacts as well as structural specificity for RNA hairpins using its conserved HTH domains. The library's diversity has been quantified at 7.84×10¹⁰.

### Example 8: Proteimer Multivalent Construct Design

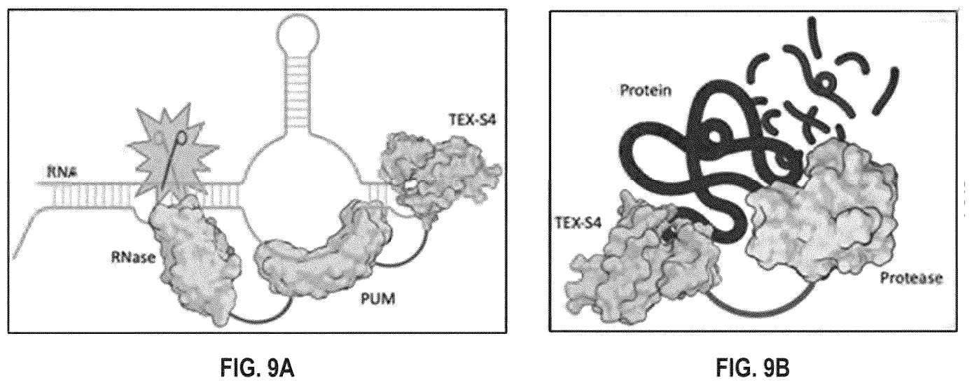

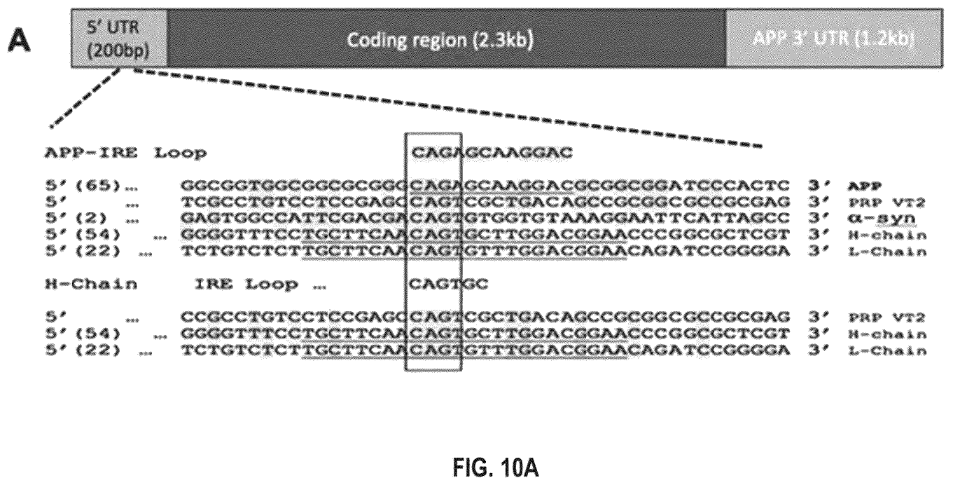

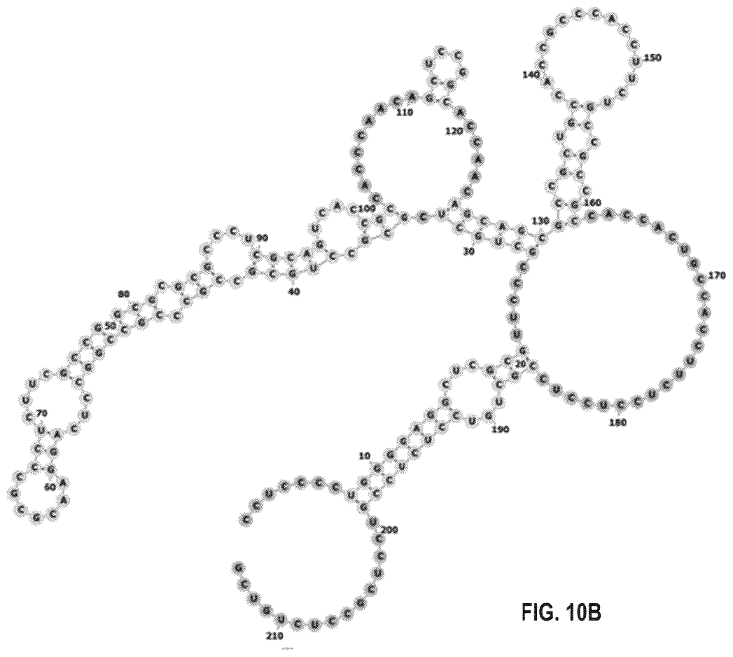

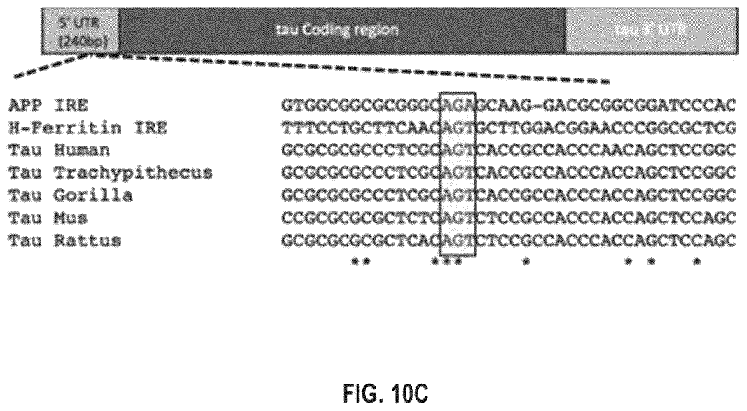

The variety of potential binding modes and targets accessible by the invention Proteimer libraries provides an additional benefit of designable multivalency. By fusing multiple Proteimer binders their strengths can be combined to further improve affinity and specificity. For example, a TEX-S4 protein which binds well to a particular RNA hairpin loop can be fused with a PUM Proteimer designed to bind an adjacent unstructured nucleotide sequence to add additional sequence specificity. Further, these multivalent constructs can be combined with functional enzymes, such as a ribonuclease (RNase) (FIG. 9A) or protease domain (FIG. 9B), and the like, to enable direct degradation of targets in addition to inhibition through binding. These multivalent constructs can also be combined with a variety of enzymatically active protein components, small molecule payloads, or stabilizing protein adducts to improve target inhibition or construct stability in physiological conditions.

### Example 9: Targeting the 5′ UTR of RNAs Using Proteimers

The repertoire of target molecules for the invention protein includes RNA, DNA, proteins, post-translational modification groups, peptides, and prosthetic groups, allowing for selective design and targeting. The 5′ UTR of RNAs (tau, SNCA, APP, ferritin, cMyc-A323M, and ARID5A), especially those with IRES or IRE structural motifs, is a promising target for the invention Proteimer platform, as these regions are essential for cap-independent translation. Similar to small molecule translational inhibitors, the invention Proteimer library can be utilized to select translational blockers for these RNA targets. Targeting the 5′ UTR of RNAs using Proteimer represents a strategic approach for regulating gene expression. By focusing on these untranslated regions, particularly those with IRES or IRE structural motifs, Proteimers can selectively inhibit translation, providing a novel method for controlling protein synthesis and potentially treating various diseases.

### Targeting the Tau 5′ UTR of RNAs Using Proteimer:

The 5′ UTR of tau mRNA plays a crucial role in regulating translation initiation through its internal ribosome entry site (IRES), which allows for cap-independent translation. The IRES-dependent translation is influenced by RNA-binding proteins and cellular stress conditions, such as elevated iron levels, poly (I:C) exposure, and extracellular amyloid-beta. A unique Iron-responsive Element (IRE) has previously been identified within the 5′ UTR of tau mRNA, suggesting that increased iron levels could enhance tau translation similarly to ferritin's L and H subunits. The small molecular translation blocker for 5′ UTR of tau mRNA co-reduce levels of tau and phosphor-tau. Targeting this 5′ UTR of tau mRNA to reduce tau protein expression provides a promising therapeutic strategy to prevent tau aggregation and treat tauopathies. Proteimers (Protein Aptamer) are provided that target the 5′ UTR of tau mRNA, specifically aiming to inhibit tau protein translation to pioneer novel therapeutic solutions against Alzheimer's disease (AD). This pharmacological strategy is designed to regulate tau expression to levels that support neuronal health, as complete tau deficiency has been linked to Parkinsonism and dementia due to impaired APP-mediated iron export.

### Example 10: Identification of Small Molecule Binders that Target the 5′UTR of SNCA mRNA by Phage Display

α-Synuclein, encoded by the SNCA gene, plays a crucial role in the development of synucleopathies, including Parkinson's disease, dementia with Lewy bodies, and multiple system atrophy. α-Synuclein is prone to aggregation and forms amyloid fibrils, spreading through the nervous system in a prior-like manner, ultimately leading to neuronal death. Although reducing α-Synuclein levels offers a promising disease-modifying strategy, targeting it presents challenges due to its intrinsically disordered nature and lack of a defined binding site for small molecules. Alternatively, α-Synuclein mRNA can directly be targeted as a strategy to lower overall levels of α-Synuclein.

The 5′UTR of SNCA mRNA features a relatively long sequence with high GC content (66%), predicted to form a stable secondary structure. A 38 bp sequence at the 5′UTR of SNCA mRNA was focused on; this sequence was predicted to form two distinct stem-loops. Using invention Proteimer libraries provided herein, high-throughput phage display screening was conducted to identify small molecule binders that target the 5′UTR of SNCA mRNA.

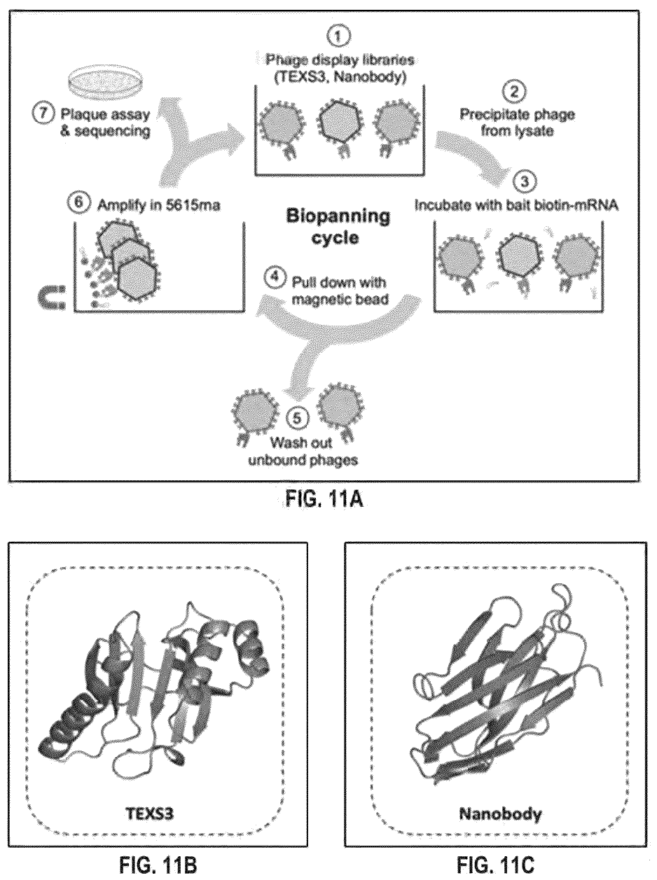

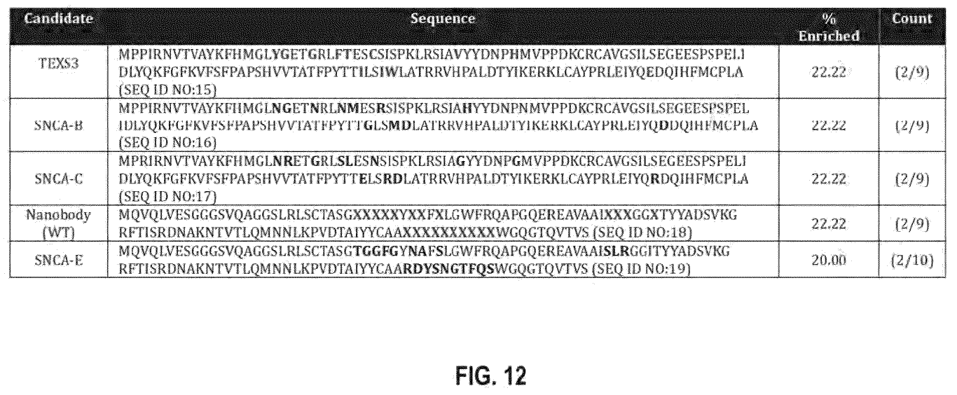

To reduce the number of non-specific binders and to prevent screening bias from components within the LB+M9 medium, phages were purified from the lysates and blocked with tRNA prior to biopanning with the 5′UTR of SNCA mRNA (FIG. 11A). As the mRNA also contained biotin at the 5′-terminus, candidate phages were enriched with streptavidin-conjugated magnetic beads which were used for succeeding rounds of biopanning, while unbound phages were washed away. Upon 6 rounds of biopanning, two sequences emerged as potential candidates from the TEXS3 library and one sequence emerged as a potential candidate from the Nanobody library (FIG. 12).

Validation of candidates: Candidates from the TEXS3 and Nanobody library screenings were cloned into the pET28b expression vector containing 6× Histidine and Sumo tags, and purified via Ni-NTA chromatography. Purified candidate recombinant proteins were subjected to kinetic evaluation by surface plasmon resonance (SPR).

SPR is an optical technique that measures molecular interactions in real time. While multicycle kinetic is the most common SPR strategy for kinetic assessment, single cycle kinetics provides an alternative method for kinetic analysis in systems where analyte binding to ligand surfaces is difficult to regenerate. The principle of single cycle kinetics relies on sequential injections of analytes of increasing concentration over the ligand without regeneration steps between each concentration, followed by a single, long dissociation phase after the highest concentration.

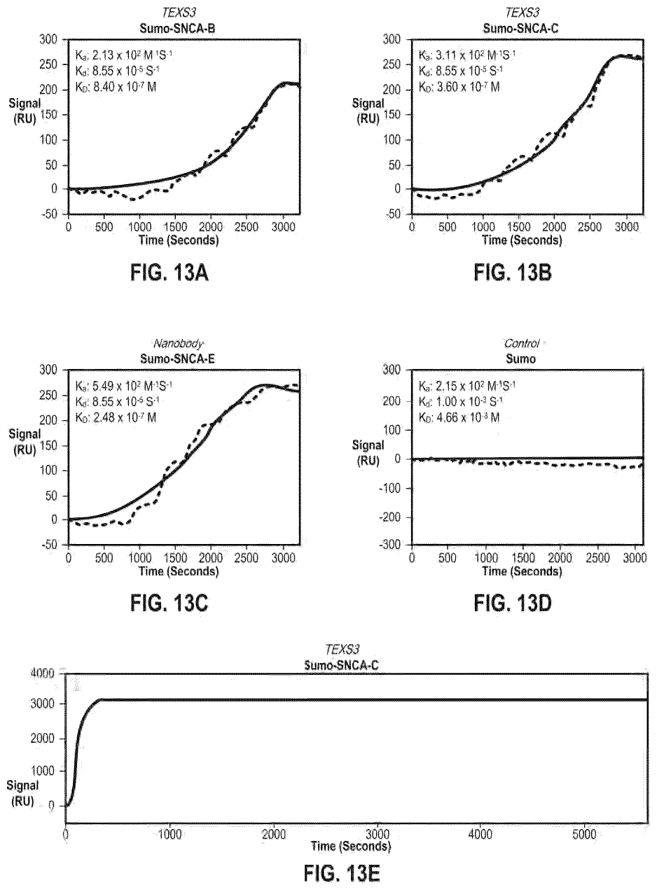

Single-cycle kinetic analysis of candidate recombinant proteins displayed high affinities to the 5′UTR of SNCA mRNA (FIG. 13A-D). Sumo-SNCA-B displayed a K_{D} of 840 nM, Sumo-SNCA-C displayed a K_{D} of 360 nM, and Sumo-SNCA-E displayed a K_{D} of 248 nM, whereas Sumo alone displayed a K_{D} of 4.66 mM. This observed high affinity was attributable to the extremely slow dissociation rates displayed by all candidates. For instance, the dissociation of Sumo-SNCA-C from SNCA mRNA was observed over 90 minutes with no change in signal (FIG. 13E). To account for the slow dissociation, the weakest possible K_{D} was calculated as if there was a 5% dissociation, following a model of a typical 600 second dissociation time with a dissociation rate (K_{d}) of 8.55E-5. Together, these results demonstrate identification of high affinity small molecule binders that targets the 5′UTR of SNCA mRNA.

Structural analysis of SNCA 5′UTR mRNA and SNCA-B, SNCA-C, and SNCA-E: RNA's functionality depends on its structural conformation and its ability to bind to a protein molecule. Precise prediction of the tertiary structure is essential to recapitulate target mRNA structure for screening high affinity Proteimers. All RNA structural predictions were evaluated through trRosettaRNA, an automated deep learning-based approach for predicting RNA 3D structures. The 5′UTR of SNCA mRNA secondary structure prediction is characterized by dominant base-paired stems and hairpin loops. Ensuing Proteimer candidates were validated for binding to target SNCA mRNA by SPR analysis (FIG. 13A-E) and subjected to molecular docking.

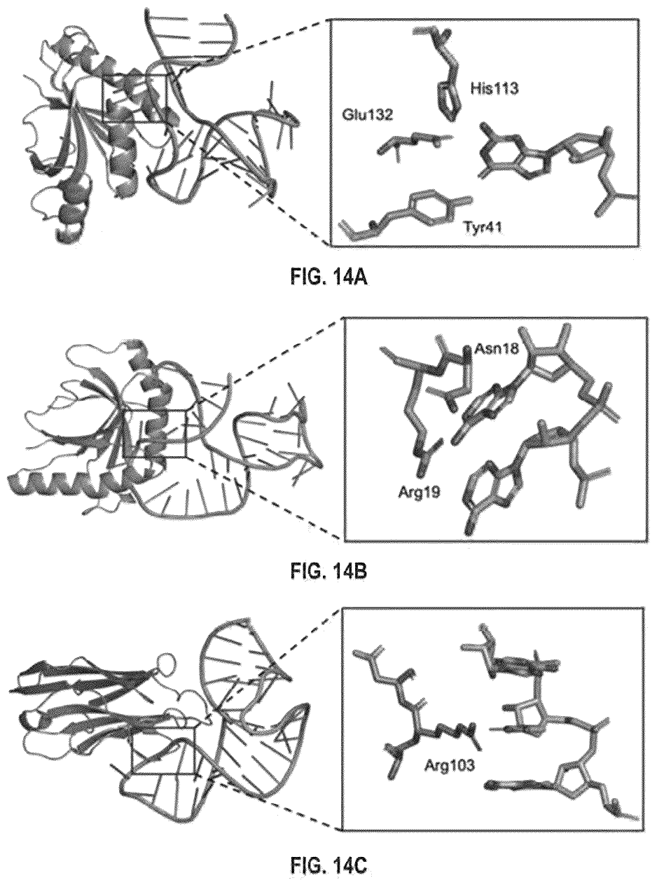

FIG. 14 presents the highest-scoring predicted binding poses of SNCA-B, SNCA-C, and SNCA-E with the 5′ UTR of SNCA mRNA, as generated by AlphaFold3. FIG. 14 highlights the interactions between these proteins and the target mRNA, providing insights into the structural alignment and potential binding mechanisms within the 5′ UTR region. SNCA-B, SNCA-C, and SNCA-E with the 5′ UTR of SNCA mRNA molecular interaction was also simulated by HDOCK. The docking simulation of SNCA-B, SNCA-C, and SNCA-E with the 5′ UTR of SNCA mRNA generated multiple potential models with high confidence scores. The docking scores were notably high at −256.22 to −312.78, calculated using HDOCK's knowledge-based iterative scoring function ITScorePP. A more negative docking score indicates a more probable binding model. Given that protein-RNA complexes in the PDB generally exhibit docking scores of approximately-200 or better, the predicted docking scores indicate highly favorable binding between SNCA-B, SNCA-C, and SNCA-E with the 5′ UTR of SNCA mRNA. These results suggest strong potential for effective interaction between the designed proteins and their RNA target.

FIG. 14 illustrates the predicted binding between SNCA-B, SNCA-C, and SNCA-E and the hairpin structure of the 5′ UTR of SNCA mRNA. A detailed interaction analysis showed that the 5′ UTR of SNCA mRNA binds within the canonical binding pockets of SNCA-B, SNCA-C, and SNCA-E, closely interacting with the designed amino acid mutations. In the top-scoring AlphaFold3 prediction, SNCA-B binds to SNCA mRNA, with a close-up inset highlighting potential stabilizing interactions involving Tyr41, His113, and Glu132 of SNCA-B with the mRNA (FIG. 14A). Similarly, SNCA-C binds to SNCA mRNA, with the inset showing interactions between Asn18 and Arg19 of SNCA-C and the mRNA (FIG. 14B). In addition, SNCA-E binds SNCA mRNA, with an inset showing an interaction between Arg103 of SNCA-E and the mRNA (FIG. 14C). All three of these examples of contacting amino acid positions are mutations from sites of variability in the designed invention Proteimer libraries. These results highlight the rational and practical design of the focused and combinatorial invention Proteimer libraries and provide demonstrated positive results for screening specific, high-affinity binders for mRNA molecules.

### Example 11: Proteimers as Catalytic Inhibitors of APP mRNA Translation: Toward a New Therapeutic for Alzheimer's Disease

The highly structured 5′ untranslated region (UTR) of APP mRNA provides a targetable element for selective inhibition using RNA-binding therapeutics. To develop and evaluate engineered protein-based RNA binders (Proteimers) that selectively target the APP 5′-UTR to inhibit translation. High-throughput phage display screening techniques were applied to identify two Proteimers with high affinity for the APP 5′-UTR, confirmed via surface plasmon resonance (SPR). Domain engineering enabled the fusion of these binders to an RNase domain to facilitate catalytic degradation of APP mRNA.

Key selected Proteimers bound the APP 5′-UTR with nanomolar affinity. Structural modeling of the Proteimer-RNA complexes revealed that the engineered mutations on the protein binding surface predominantly interact with the APP mRNA, with the 5′-AGA-3′ region folding outward into the binding pocket. RNase-fused Proteimers mediated sequence-specific APP mRNA cleavage in vitro, demonstrating robust target engagement and degradation. The Proteimer ProAPPS3-11 effectively inhibited APP translation in SH-SY5Y cells, reducing protein levels by up to 60% in a dose-dependent manner. These findings establish the feasible use of Proteimers as a novel class of RNA-targeting biologics with therapeutic potential to reduce APP levels and APP mRNA levels, thus to mitigate downstream AD-related neurodegeneration.

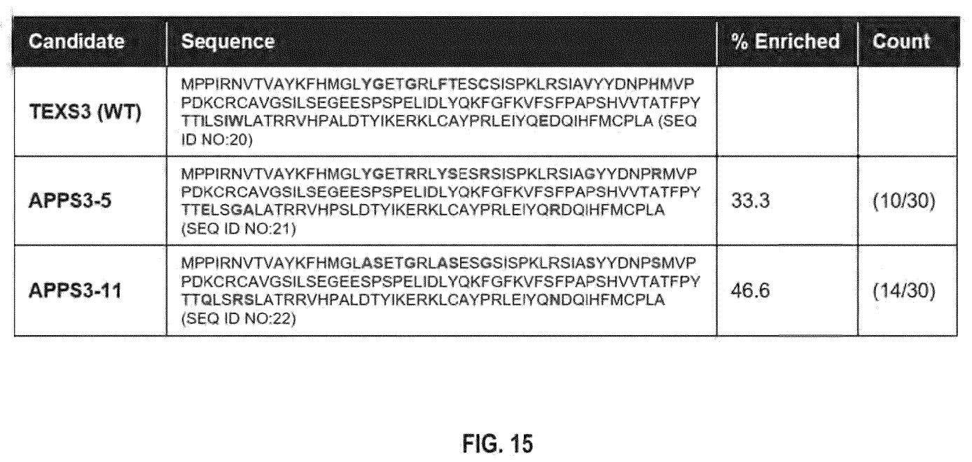

Discovery of proteins that bind APP 5′-UTR by phage display: Biopanning utilizing the T7 phage display system in conjunction with the developed TEXS3 library was performed to identify any possible binders to the 5′-UTR containing the IRE motif of the APP mRNA. The T7 Phage display system was used with the 5615rna cell line to avoid degradation of RNA by native RNAses. A biotinylated version of the 5′-UTR APP mRNA was utilized as a bait to target the TEXS3 protein from the constructed library of 5.58×10¹⁰ species being expressed on the phage surface. The APP bait-phage mixture was enriched with streptavidin-conjugated magnetic beads to capture the successfully bound phages, while unbound phages were washed away. Upon 8 rounds of biopanning, two sequences emerged as potential candidates, namely APPS3-5 and APPS3-11 (FIG. 15).

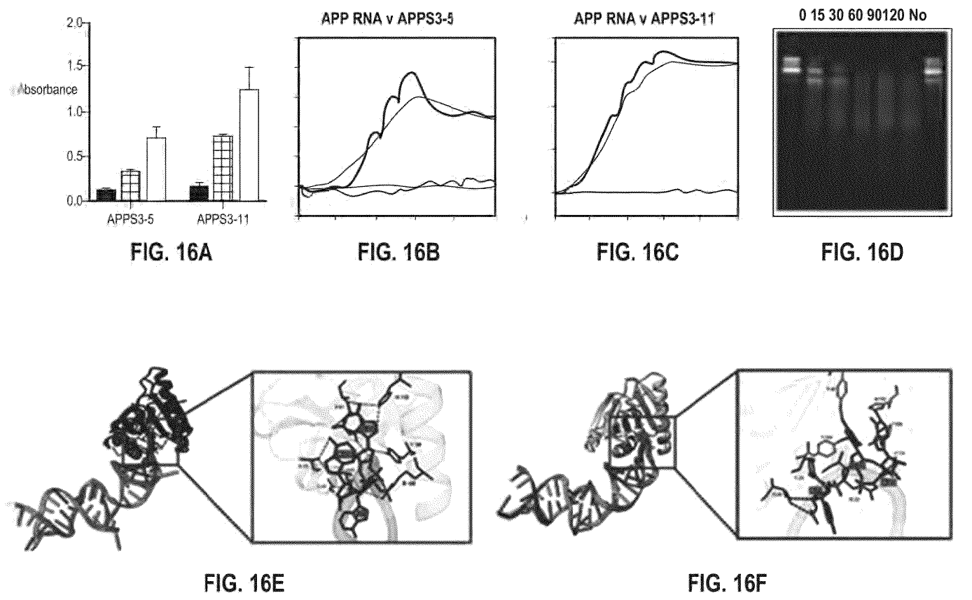

SPR results validate candidates: The two biopanning positive candidates, APPS3-5 and APPS3-11, that demonstrated binding to the APP mRNA were cloned into a pET-28b expression vector with an N-terminal 6×-histidine and a SUMO tag to be purified through nickel affinity chromatography. The purification led to a highly purified product that could be utilized to study the binding and kinetics of each candidate with greater detail. Initial binding capabilities were re-evaluated using an ELISA where the purified APPS proteins were adhered into the wells of a 96-well plate. The biotinylated APP mRNA was then applied to each well at varying concentrations and binding was confirmed using a streptavidin-HRP conjugate. The results showed an increase in signal over the concentration gradient confirming high binding affinity of these candidates (FIG. 16A).

Upon verification of binding affinity through ELISA, candidates were further tested for kinetics utilizing SPR. SPR can be used to measure the real time binding interactions between a bound ligand and analyte pair. While multicycle kinetic analysis is the most common assessment utilized to measure kinetic binding through SPR, single cycle analysis has proven to be reliable in understanding the binding kinetics in systems where analyte dissociation and sensor regeneration are not possible. Single-cycle kinetic analysis of the two candidates, APPS3-5 and APPS3-11, displayed high affinities primarily to the folded 5′-UTR of APP mRNA bait where APPS3-5 displayed a K_{D} of 176 nM and APPS3-11 displayed a K_{D} of 7.3 nM (FIG. 16B). Both candidates showed high binding affinity likely due to the extremely slow dissociation rates where little to no change in signal was observed over a 90 minutes time interval. However, this binding signal could only be seen when utilizing a chip bound with folded APP mRNA.

When the APP mRNA bait is not folded prior to binding to the SPR chip, no signal is obtained when running the APPS proteins indicating the need for secondary structure to allow for binding to occur. Binding to the APP mRNA could also only be seen when utilizing the APPS Proteimers when compared to non-APP mRNA PROTIEMERS. Previously designed TEXS3 based binders that were specific to other mRNA targets, such as the 5′-UTR of the SNCA mRNA, showed no binding signal (FIG. 16C). These results along with the gathered ELISA data demonstrate the possibility of developing protein-based high affinity RNA binders capable of targeting the 5′-UTR IRE motif of APP mRNA.

Structure prediction of biopanning candidates to APP IRE mRNA motif: The SPR results had validated that each of the chosen biopanning candidates were capable of binding to the IRE domain of the APP mRNA. To further verify how this binding may occur, structure prediction modeling was performed to generate models of the protein-RNA interaction between each biopanning candidate to the IRE motif of the APP mRNA. These models were generated using the Alphafold3 webserver by inputting the amino acid sequences of each candidate and pairing them with the 50-bp IRE motif of the APP mRNA. Several rounds of each prediction were performed and compared against each other to determine where binding would most likely occur. Through these prediction models, both biopanning candidates APPS3-5 and APPS3-11 appeared to bind to the target APP within the canonical ligand-binding cleft of its Gyrl-like domain.

The designed mutations incorporated into the protein binding surface of the candidates predominately form this interaction with the APP mRNA, where the 5′-AGA-3′ region can be seen to fold outward into the binding pocket (FIG. 16E-F). This 5′-AGA-3′ region coincides with a unique alternative RNA triloop formation. This set of nucleotides coincides with key IRE motifs and is predicted to form a bulge-like structure with the 5′-UTR APP mRNA. These structural predictions provide a general insight into understanding how these interactions may contribute to the affinity and specificity between protein and mRNA.

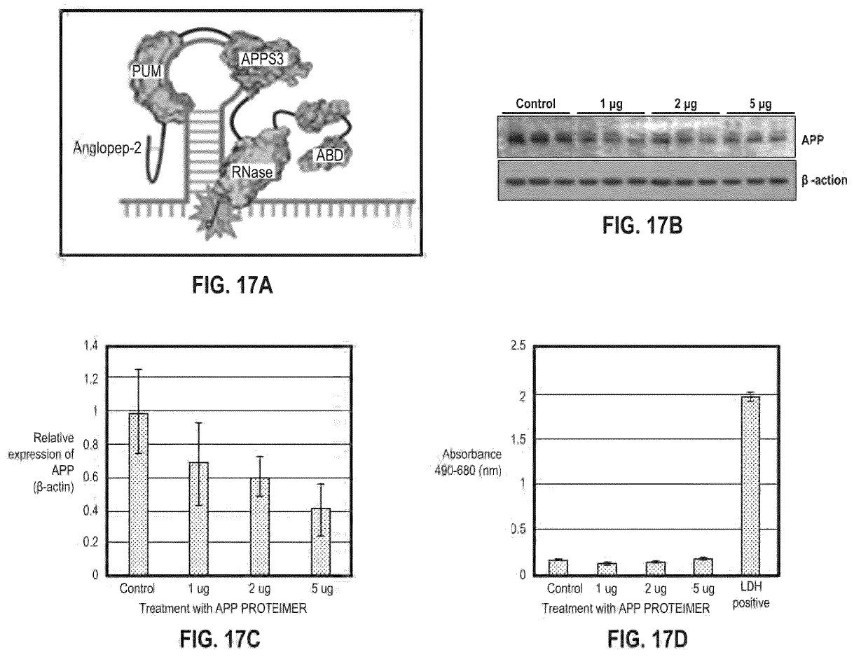

Candidates can target the degradation of RNA in vitro: After confirming that the biopanning candidates could bind to the APP RNA, their capacity to promote RNA degradation was evaluated. New large fusion proteins were designed to incorporate these RNA-binding domains (APPS3-5, APPS3-11) with multiple secondary protein domains each providing unique functions to the fusion protein as a whole to aid in this role of targeted RNA degradation (FIG. 17A). A C-terminal PIN RNA endonuclease (amino acids 553-734 of SEQ ID NOs: 176 and 177) was added to provide the catalytic activity needed to cleave the RNA upon binding. To enhance sequence specificity, a PUM1-derived PUF domain (amino acids 27-376 of SEQ ID NOs: 176 and 177) was incorporated to bind a region immediately downstream of the 5′-UTR hairpin targeted by the candidate constructs. For in vivo application, the design was further optimized by adding an N-terminal Angiopep-2 peptide (amino acids 3-21 of SEQ ID NOs: 176 and 177) to enable transcytosis across the blood-brain barrier and entry into neurons, along with two C-terminal albumin-binding domains (ABD) (2 ABD domain at amino acids 745-790 and 801-846 of SEQ ID NOs: 176 and 177) to prolong systemic circulation and improve pharmacokinetic stability.

The delivery of protein-based therapeutics to the CNS remains a major challenge due to the protective nature of the blood-brain barrier (BBB) and limited uptake by neuronal cells. To overcome this, Angiopep-2 peptide was appended to the N-terminus of ProAPP-S3-5, ProAPP-S3-11 constructs. These motifs exploit receptor-mediated transcytosis (RMT) pathways via LRP1, respectively-well-characterized strategies for CNS delivery. As LRP1 is abundantly expressed at the BBB and in neurons, it offers an effective route for delivering therapeutics into the brain and neuronal cells via receptor-mediated endocytosis (RME).

The final constructs ProAPPS3-5 (SEQ ID NO: 176), and ProAPPS3-11 (SEQ ID NO: 177) were expressed, purified, and tested for their ability to degrade an in vitro-transcribed segment of APP RNA. The PUM-TEX candidate-PIN construct was incubated at a 3 μM (ProAPPS3-11) or 4.5 UM (ProAPPS3-5) concentration with the APP RNA for 0-120 minutes at 37° C. and the RNA from the reactions was analyzed by polyacrylamide gel electrophoresis. Over time, the band for the full-length RNA diminished in intensity, then disappeared, while bands/a smear for RNAs of shorter lengths appeared, indicating that the RNA was being targeted by the RNase fusion construct (FIG. 16D). ProAPPS3-11 was able to degrade the APP RNA more quickly and completely than ProAPPS3-5, although both constructs were active. RNA incubated without protein did not degrade significantly over time, confirming that the construct with the RNase was responsible for degradation.

Confirmation of APP target engagement and silencing in SH-SY5Y neuroblastoma cells: To assess functional target engagement, SH-SY5Y neuroblastoma cells, which endogenously express APP, were treated with the multifunctional Proteimer construct, ProAPPS3-11 (SEQ ID NO:177). This engineered fusion protein incorporates modular domains to optimize delivery and efficacy: Angiopep-2 for BBB and neuronal penetration (amino acids 3-21 of SEQ ID NO: 177), a PUM domain for sequence-specific RNA recognition (amino acids 27-376 of SEQ ID NO:177), APPS3-11 invention Proteimer for high-affinity RNA binding (amino acids 392-537 of SEQ ID NO: 177), an PIN RNase domain for catalytic RNA degradation (amino acids 553-734 of SEQ ID NO:177), and an albumin-binding domain (ABD) (2 ABD domain at amino acids 745-790 and 801-846 of SEQ ID NO: 177) to extend systemic half-life (FIG. 17A). These modular domains are also present at the same locations in invention ProTex-T1 (SEQ ID NO: 180) set forth herein. Between each of these domains are peptide linker sequences that vary in length represented by the following formula:

Start-Angiopep2-Linker-PUM-Linker-Proteimer-Linker-PIN-Linker-2×ABD-TEV6×His-Stop

As an initial evaluation of biological activity, ProAPPS3-11's ability to inhibit APP translation was examined in a dose-dependent manner (FIG. 17B-C). The construct was specifically designed to bind the A-bulge near the base of the IRE hairpin within the 5′-UTR of APP mRNA. Human SH-SY5Y neuroblastoma cells remained healthy throughout the experiment, comparable to untreated controls. Western blot analysis performed after 48 hours of treatment with pcDNA3 control or increasing concentrations (1, 2, and 5 μg) of the pcDNA3-ProAPPS3-11 plasmid revealed a clear dose-dependent reduction in APP protein levels. APP expression was normalized to β-actin, which served as a loading control, confirming the specificity and consistency of the observed translational repression (FIG. 17B-C).

At the highest concentration tested, APP expression was reduced by approximately 60%, confirming effective and selective translational repression of APP by ProAPPS3-11 (FIG. 17B-C). In order to test whether ProAPPS3-11 treatment had a detrimental effect on cells, an LDH cytotoxicity assay was used. In the assay, all cell treatments had similar low corrected absorbance readings at 490 nm compared to the LDH positive control supplied by the kit. This indicated that, even at the highest dose of the Proteimer, there was minimal cytotoxicity (FIG. 17D).

### Example 12: Targeting the Tau 5′ UTR with Proteimer as a Therapeutic Strategy

Phage display screening of tau 5′ UTR-binding Proteimer candidates: To identify high-affinity binders against the 5′ untranslated region (UTR) of tau mRNA, biopanning was performed using the T7 phage display system in the E. coli 5615rna strain, which lacks endogenous RNases to minimize RNA degradation. To reduce non-specific interactions and mitigate background binding from components of the LB+M9 growth medium, phages were pre-blocked with yeast tRNA following purification from crude lysates. Biotinylated tau 5′ UTR RNA (30 nucleotides) was immobilized on streptavidin-conjugated magnetic polystyrene beads and used as bait for selection.

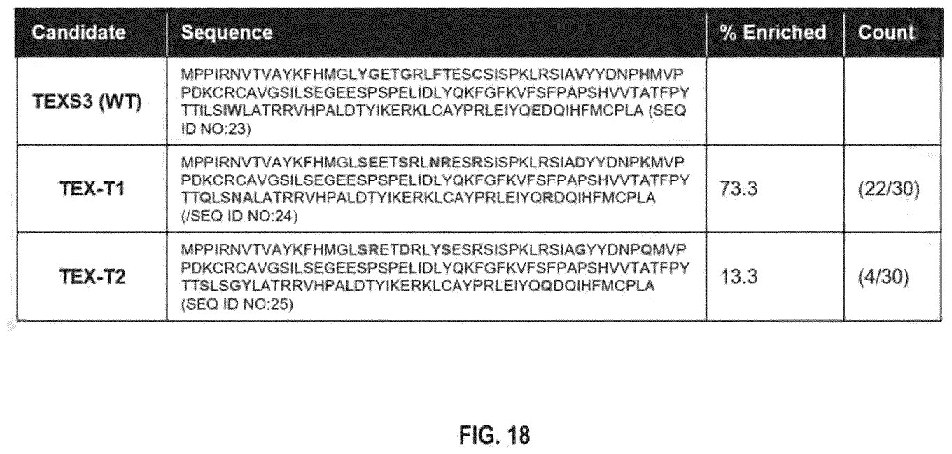

Unbound phages were removed by iterative washing, while bound phages were eluted and amplified for subsequent rounds of selection. Biopanning was carried out over six rounds against both a focused TEX264-derived Proteimer library (TEXS3) and a nanobody library (FIG. 18). To increase selection pressure and enrich for high-affinity binders, the concentration of RNA bait was progressively decreased from 100 nM to 10 nM across the selection rounds. Following the final round, phage DNA was isolated and sequenced. One dominant clone was identified from the TEXS3 library and one from the nanobody library (FIG. 18), both of which were prioritized for subsequent biochemical validation and characterization.

Validation of tau 5′ UTR-binding Proteimer candidates: Two lead invention Proteimer candidates-TEX-T1 from the TEXS3 library and Nano-T1 from the nanobody library—were identified based on strong sequence convergence following six rounds of phage display selection. These candidate sequences were cloned into the pET28b expression vector, which includes N-terminal 6×His and SUMO fusion tags to facilitate expression and purification. Recombinant proteins were purified by Ni-NTA affinity chromatography and confirmed by SDS-PAGE. To assess binding affinity and kinetic parameters, surface plasmon resonance (SPR) was employed using a single-cycle kinetic (SCK) approach. SCK is particularly advantageous for analyte-ligand systems that are difficult to regenerate, permitting the measurement of binding kinetics through sequential injections of increasing analyte concentrations followed by a single extended dissociation phase.

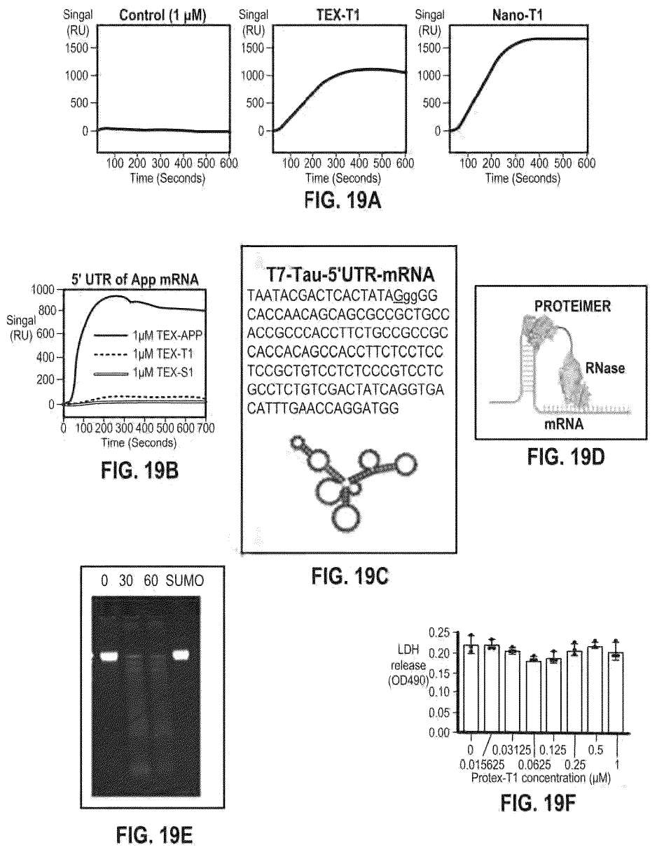

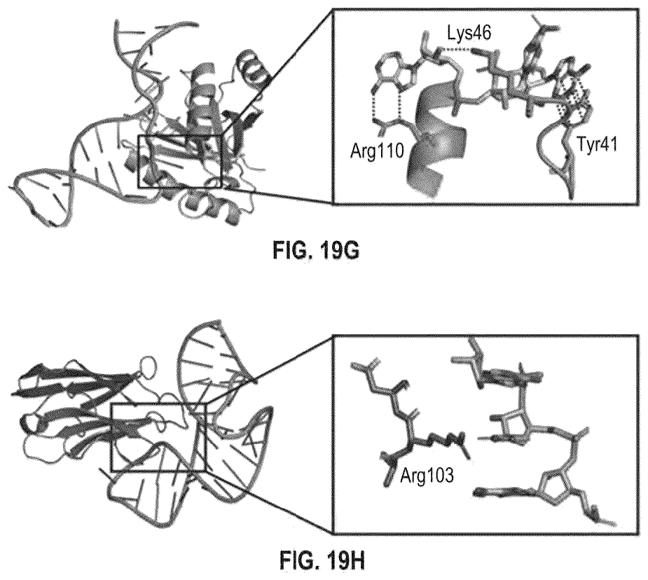

SPR analysis revealed that both candidate proteins specifically bind the tau 5′ UTR with high affinity (FIG. 19A-B). SUMO-TEX-T1 exhibited a dissociation constant (K_{D}) of 740 nM, and SUMO-Nano-T1 showed a K_{D} of 215 nM. In contrast, the SUMO tag alone exhibited significantly weaker binding (K_{D}=4.66 mM), confirming the specificity of the engineered binders (FIG. 19A-B). The high binding affinity of TEX-T1 and Nano-T1 was attributed to exceptionally slow dissociation rates. Notably, no measurable signal decrease was observed over a 90-minute dissociation phase for SUMO-TEX-T1. To estimate the minimal possible K_{D} under these conditions, a hypothetical 5% dissociation over 600 seconds was modeled, yielding a dissociation rate constant (k_off) of 8.93×10⁻⁵⁴. Collectively, these results validate TEX-T1 and Nano-T1 as high-affinity, sequence-specific RNA-binding proteins targeting the IRE within the 5′ UTR of tau mRNA. Among previously developed Proteimers targeting other baits (e.g., APP and SNCA mRNAs), only the tau-specific TEX-T1 selectively bound the 5′UTR of tau mRNA, but not APP mRNA (FIG. 19A-B), demonstrating high target specificity. These findings support the potential of TEX-T1 as a selective translational inhibitor of tau.

Targeted degradation of tau mRNA using a Proteimer-RNase fusion construct: Following confirmation that the identified RNA-binding proteins (RBPs) exhibited high-affinity binding to the 5′ UTR of tau mRNA, their potential for targeted mRNA degradation was evaluated. For these experiments, the SUMO-tagged TEX-T1 candidate was selected from the TEXS3 library and engineered into a fusion construct by appending a catalytically active RNase domain to its C-terminus. The resulting SUMO-TEX-T1-RNase fusion protein was expressed and purified using the same protocol as described for the original candidate. To assess the RNA-cleaving activity of the fusion construct, an in vitro transcribed mRNA containing the 5′ UTR of tau was synthesized as the degradation substrate (FIG. 19C).

The transcript was incubated with 3 UM of the SUMO-TEX-T1-RNase fusion protein for 0 to 60 minutes. RNA integrity was evaluated by electrophoresis on a 15% urea-TBE denaturing polyacrylamide gel. Over the course of the incubation, progressive degradation of the full-length transcript was observed, accompanied by the accumulation of smaller RNA fragments (FIG. 19E), indicating time-dependent, sequence-specific RNA cleavage by the fusion construct. To exclude non-specific degradation due to contaminating RNases or spontaneous hydrolysis, control reactions were performed using the SUMO tag alone. No significant degradation of the tau 5′ UTR transcript was observed in the control reactions, confirming that RNA cleavage was specifically mediated by the RNase domain fused to the TEX-T1 candidate (FIG. 19D-E). These results support the utility of Proteimer-based RNA-binding proteins as platforms for targeted RNA degradation strategies.

Structural analysis of tau 5′ UTR mRNA-candidate interactions: To elucidate the binding mechanisms of the identified Proteimer candidates, in silico protein-RNA complex structure prediction was conducted using the amino acid sequences of TEX-T1 and Nano-T1 in combination with the nucleic acid sequence of the tau 5′ UTR iron-responsive element (IRE) region used for biopanning. For each candidate, the top-ranked protein-RNA complex from the predictive models was selected for structural analysis. Both TEX-T1 and Nano-T1 were predicted to engage the tau mRNA via designed residues on their RNA-binding interfaces, primarily through polar interactions between side chains and nucleobases.

These interactions appear to mimic canonical base-pairing, contributing to target specificity and affinity (FIG. 19). The TEX-T1 candidate, derived from the Gyrl-like domain of the human TEX264 scaffold, binds RNA within its native ligand-binding cleft. The tau mRNA nucleotides adopt an “open” conformation, flipping into the binding pocket to enable tight contact (FIG. 19G-H). Detailed interaction mapping revealed that TEX-T1 specifically recognizes the conserved 5′-AGU-3′ motif of the tau 5′ UTR. Within the binding cleft, Arg110 forms pseudo-base-pairing hydrogen bonds with the mRNA, while Tyr41 engages in TT-TT stacking with exposed bases (FIG. 19G). Additionally, Lys46 is positioned to interact with the ribose moiety or phosphate backbone of the mRNA via hydrogen bonding. Notably, all three contact residues—Arg110, Tyr41, and Lys46—originate from variable positions introduced in the TEX Proteimer library design, suggesting successful evolution of functional binding surfaces through the selection strategy.

In contrast, the nanobody-based binder Nano-T1 exhibits a distinct mode of RNA recognition. Rather than relying on single-stranded interactions, Nano-T1 is predicted to engage both the stem and loop regions of the tau mRNA hairpin. Its engineered CDR loops intercalate within the RNA's structured grooves, stabilizing the complex through multiple polar contacts (FIG. 19H). This binding mode suggests a broader interaction footprint and highlights the structural versatility of nanobody-derived Proteimers. These predictive models provide mechanistic insights into how Proteimers engage structured RNA targets with high affinity and specificity. Future structural validation by methods such as cryo-EM will be critical to confirm these binding modes and guide further optimization.

Tau target engagement and suppression of translation in SH-SY5Y neuroblastoma cells: To assess functional target engagement, SH-SY5Y neuroblastoma cells—which endogenously express tau—were treated with the multifunctional Proteimer construct, ProTEX-T1 (SEQ ID NO:180). This engineered fusion protein incorporates modular domains to optimize delivery and efficacy (at the respective locations set forth above for SEQ ID NOs: 176 and 177): Angiopep-2 for blood-brain barrier (BBB) and neuronal cell penetration, a PUM domain for sequence-specific RNA recognition, TEX-T1 for high-affinity RNA binding, an RNase domain for catalytic RNA degradation, and an albumin-binding domain (ABD) to extend systemic half-life. Human SH-SY5Y neuroblastoma cells remained healthy throughout the experiment, as evidenced by their morphology and LDH assay results, which were comparable to untreated controls. (FIG. 19F). The dose-dependent inhibitory effect of ProTEX-T1 on tau protein production was evaluated, normalizing protein levels to β-actin. Treatment with ProTEX-T1 resulted in approximately a 50% reduction in tau expression, demonstrating its effective and selective translational repression of tau in SH-SY5Y neuroblastoma cells.

### Example 13: Targeting the CD19 with Proteimer as a Therapeutic Strategy

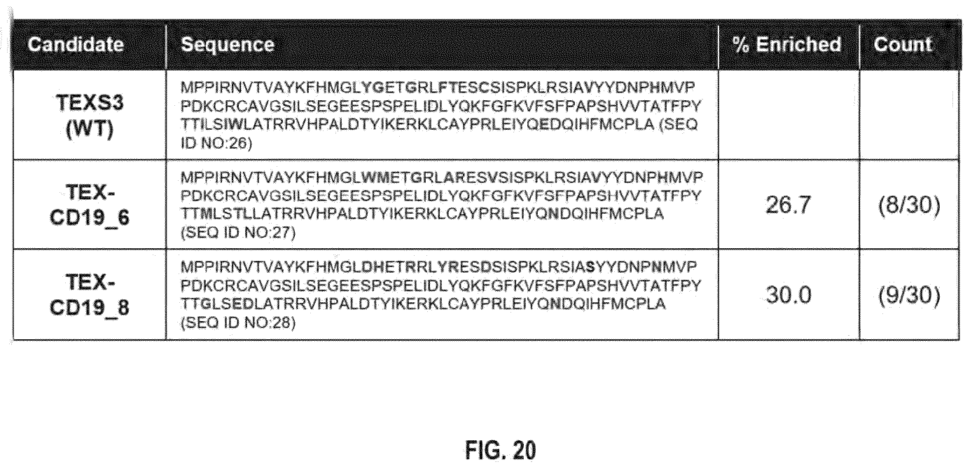

Selection of CD19-binding Proteimers via phage display: To isolate CD19-specific binders, iterative biopanning was performed using the T7 phage display system and recombinant biotinylated human CD19 protein. Phages displaying library members were pre-cleared using BSA to reduce nonspecific interactions and enriched using streptavidin-conjugated magnetic beads. After 8 rounds of selection, two distinct TEXS3-derived sequences (TEX-CD19_6 and TEX-CD19_8) emerged as high-confidence CD19 binders (FIG. 20). These clones were prioritized for further validation.

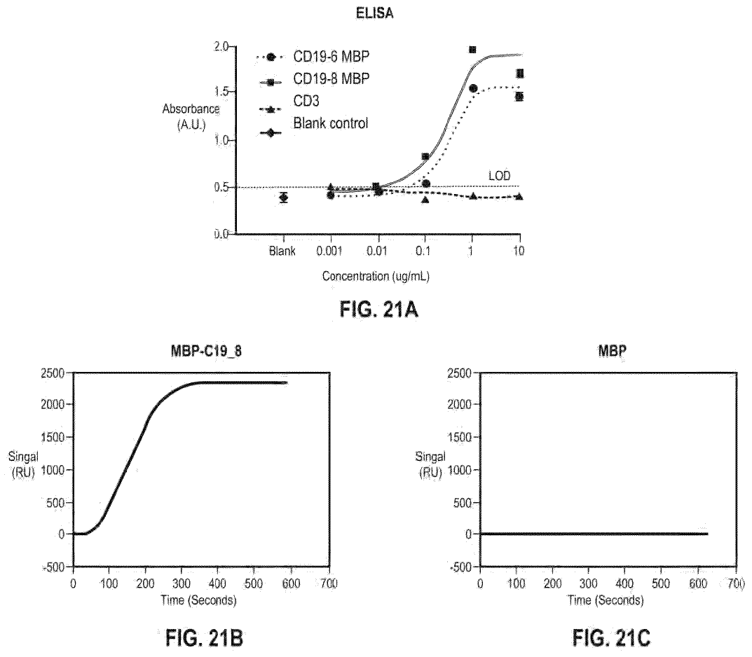

Recombinant expression and affinity validation via ELISA and SPR: Candidate Proteimers were cloned into pET28b vectors containing a 6×His tag and either MBP fusion partners, then expressed in E. coli and purified using Ni-NTA affinity chromatography. To assess binding capabilities, enzyme-linked immunosorbent assays (ELISAs) were performed (FIG. 21A). Purified MBP-TEX-CD19_6 and MBP-TEX-CD19_8 proteins were immobilized in 96-well plates, followed by the addition of varying concentrations of biotinylated CD19 protein. Binding was detected using a streptavidin-HRP conjugate, and results demonstrated a concentration-dependent increase in signal, confirming strong affinity of these candidates (FIG. 21).

Following ELISA validation, surface plasmon resonance (SPR) was employed to characterize binding kinetics using a single-cycle analysis of the SPR data was performed using TraceDrawer under 1:1 binding model. This method is particularly effective when analyte dissociation or sensor regeneration is challenging. SPR results showed that both MBP-TEX-CD19_6 and MBP-TEX-CD19_8 exhibited high-affinity binding to CD19, with dissociation constants (K_D) of 360 nM and 840 nM, respectively (FIG. 21B). In contrast, a negative control MBP construct showed significantly weaker binding (K_D=4.66 mM) (FIG. 21C). The strong binding observed for the candidates was largely attributed to their slow dissociation rates, indicating stable and durable interactions.

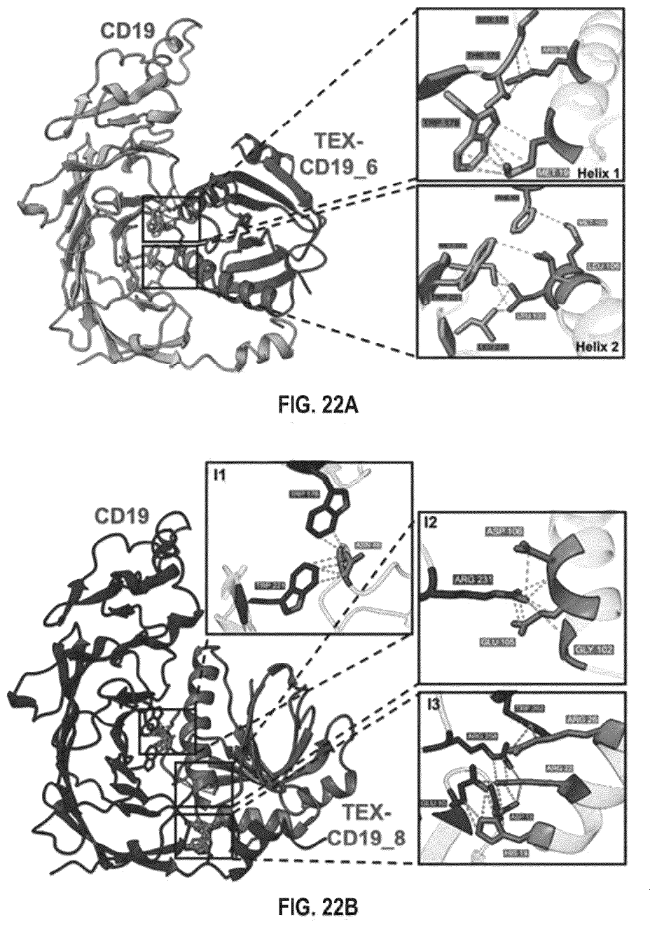

Structural Modeling of CD19-Protein Complexes: The ELISA and SPR results had validated that both of the chosen biopanning candidates were capable of binding to the CD19 protein (FIG. 22). To elucidate the molecular basis of CD19 recognition, structural predictions of Proteimer-CD19 complexes were generated using AlphaFold-based modeling and docking. In all cases, the predicted binding interface localized to the engineered surface of the TEX264 Gyrl-like domain, consistent with the intended design (FIG. 22). The models revealed strong polar interactions and complementary surface topography, supporting specific engagement of CD19 within the canonical ligand-binding cleft of the Proteimer scaffold (FIG. 22).

Notably, structural models of TEXS3 variants TEX-CD19_6 and TEX-CD19_8 demonstrated distinct sets of mutated residues forming close-range interactions (<3.5 Å) at critical binding sites, further stabilized by aromatic stacking and key hydrogen bonds within the CD19 interface (FIG. 22). Aromatic residues from CD19 seem very likely to stabilize the TEX-CD19_6 binding pocket. TEX-CD19_8 could be seen to bind more prominently to the bottom half of the CD19 cleft (FIG. 22). These insights provide a structural explanation for the high-affinity binding observed experimentally and offer a basis for structure-guided affinity maturation and therapeutic optimization.

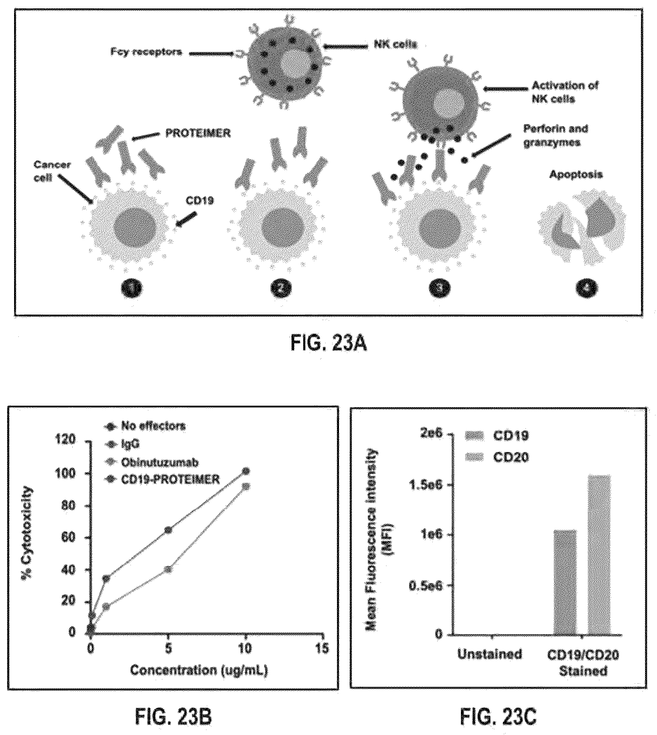

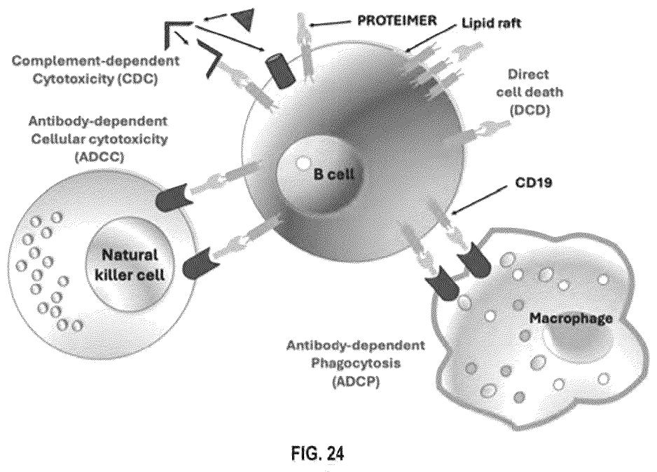

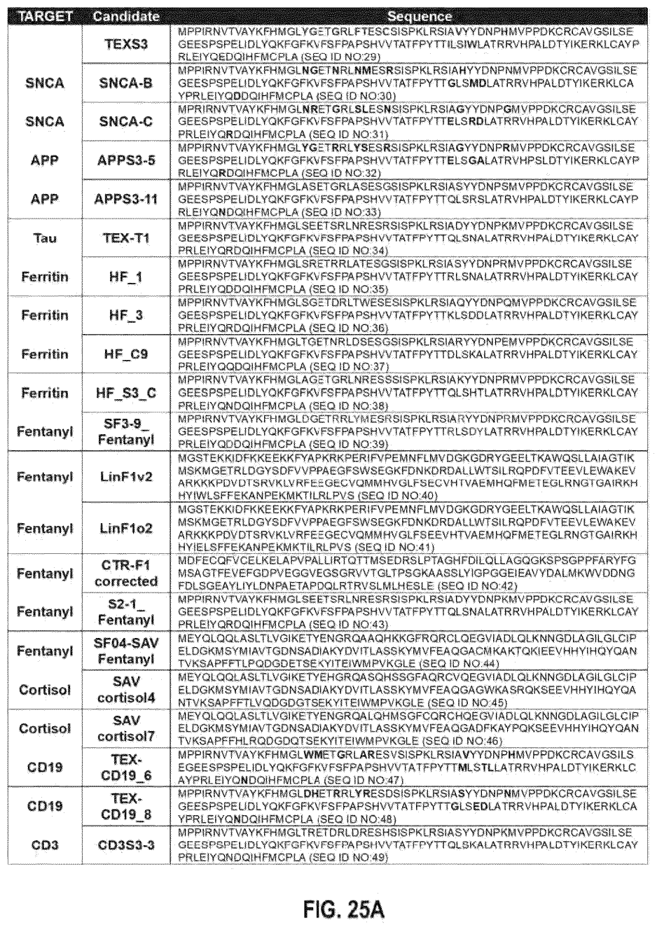

ADCC activity of CD19-PROTEIMER-Fc fusion proteins In vitro: In order to determine the functional impact of the CD19-binding Proteimer, an ADCC assay was performed with TEX-CD19_8-Fc chimeric Proteimer (SEQ ID NO:178) using normal donor NK cells and the OSU-CLL cell line as the target. The data shows that the TEX-CD19_8 induces robust ADCC at a level consistent with the CD20 targeting antibody obinutuzumab (FIG. 23B), although the level of CD20 expression on OSU-CLL was consistently higher than CD19 (FIG. 23C). It had previously been found that CD19 targeting antibodies have induced only modest levels of ADCC, therefore the translational potential pf the CD19-binding Proteimer is extremely promising. Also, provided herein is TEX-CD19_6-Fc chimeric Proteimer corresponding to SEQ ID NO: 179.

### Example 14: Targeting the Other Various Targets with Proteimer

Selected Proteimers against multiple targets using phage display are provided herein. To isolate cancer-specific binders and other target-specific candidates, iterative biopanning was performed with the T7 phage display system and recombinant biotinylated human target proteins. This approach produced biopanning-derived TEXS3 variants and nanobodies targeting a diverse set of molecules, including 5′UTR mRNA motifs of SNCA, APP, Tau, Ferritin, cMyc A323M, and ARID5A, as well as fentanyl, cortisol, CD19, CD3, CD33, CD37, HuR, CD20, CD22, Bcl-2, Her2, and APP-derived peptides. In addition, TED variants were isolated against TAAR1, TAAR1-ECL2, TAAR1-ECL1, CD37, CD3, CD20, and CD22.

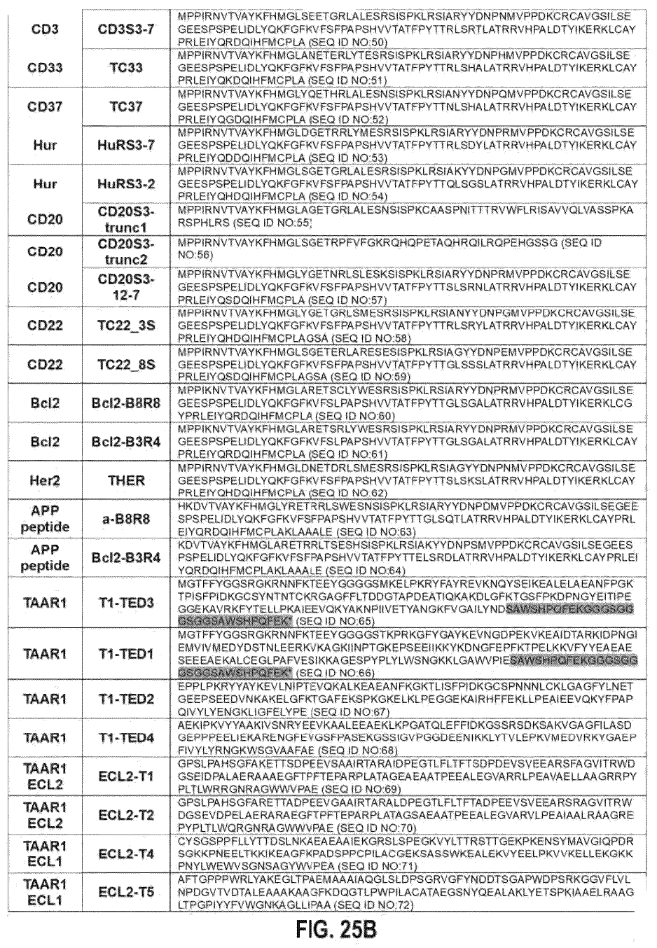

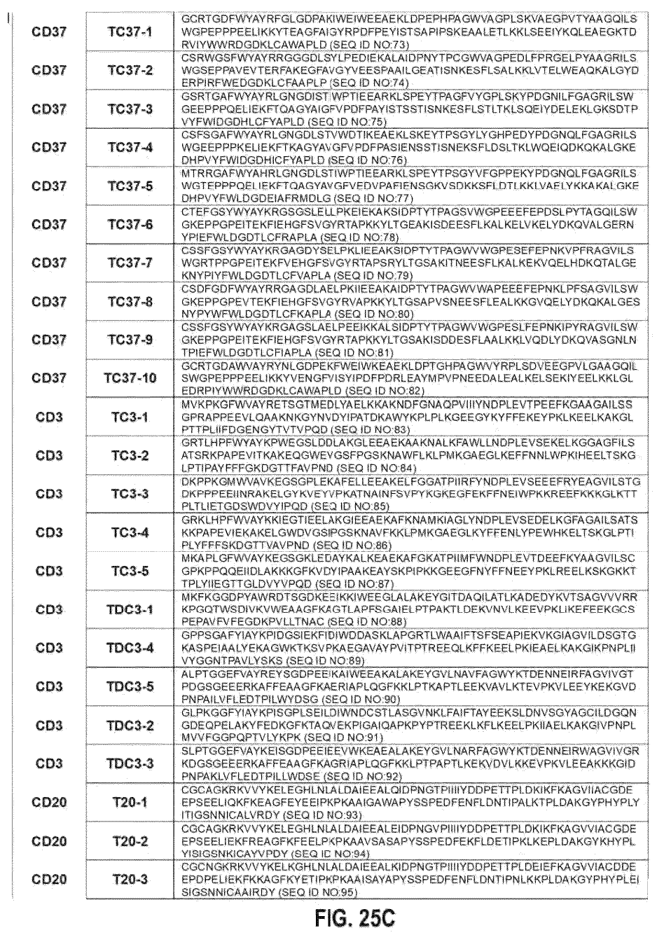

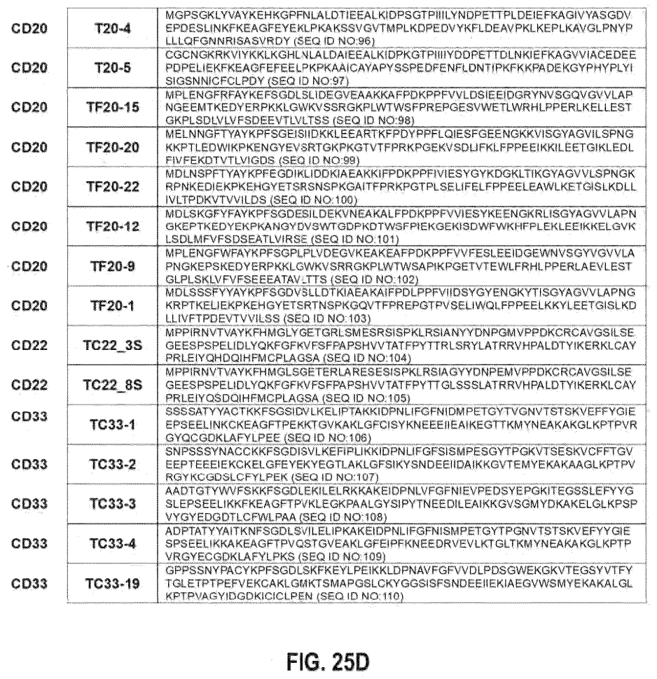

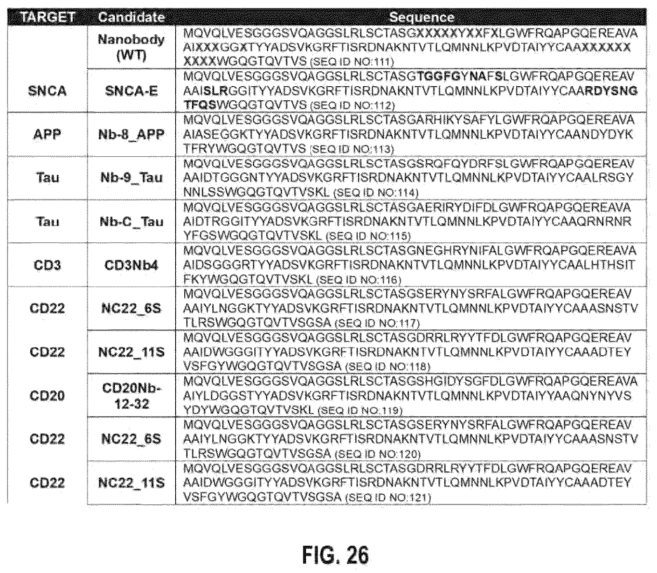

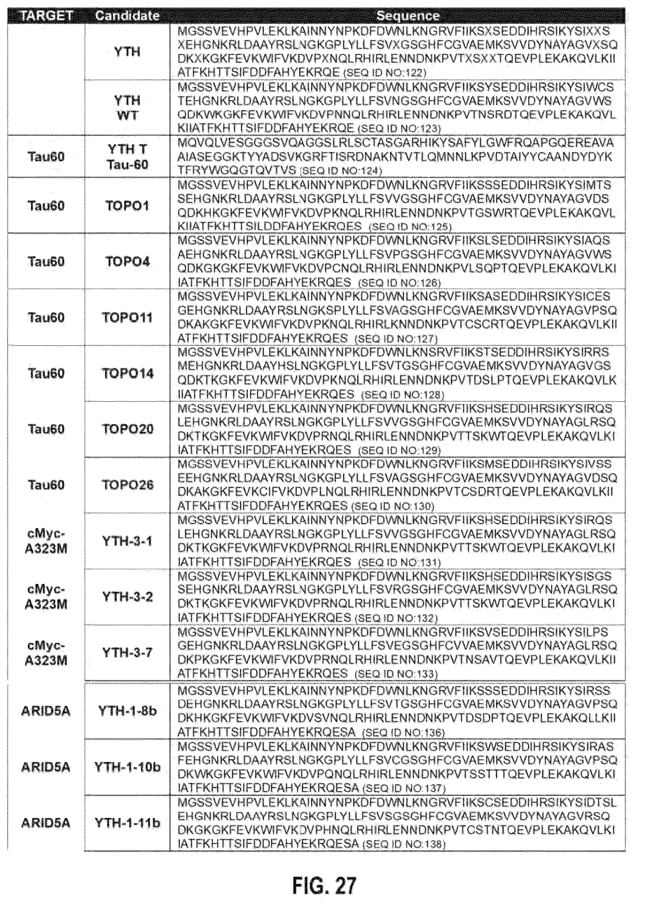

Following 8 to 12 rounds of selection, candidate sequences derived from TEXS3 (FIG. 25), nanobody scaffolds (FIG. 26), and TED (Modified form TEX) libraries (FIG. 27) consistently emerged as high-confidence binders to their respective targets.

### Example 15: Screening Gyrl-Like Aptamers for High Affinity Binding to SARS-COV-2 Spike Protein RBD

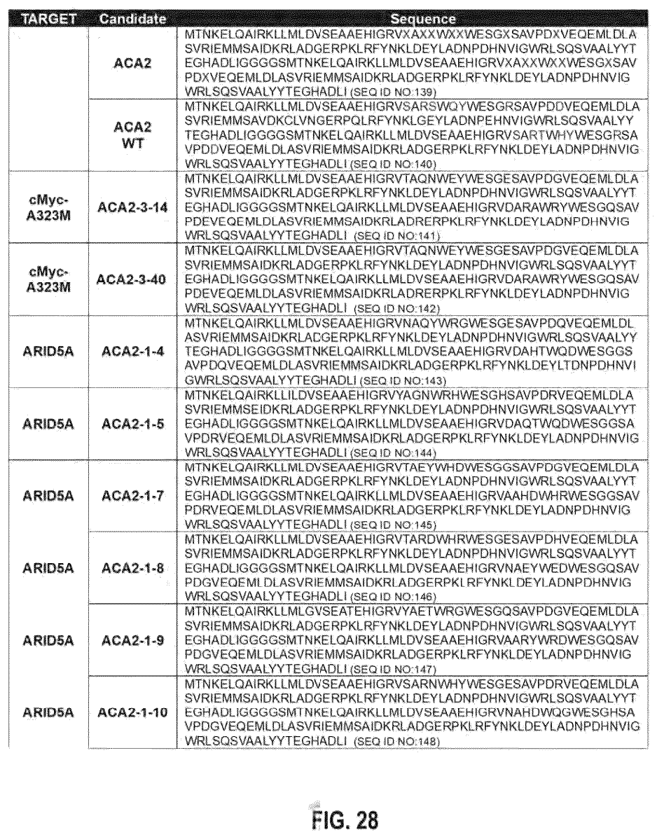

A refinement of the phage display protocol to include more stringent washes and more rounds of selection drastically reduced the number of unique positive recombinants during plaque analysis. With this new protocol a novel Gyrl-like aptamer from the SAV2435 library was identified through 8 rounds of high stringency selection. This aptamer termed SAV HS along with rationally designed aptamers were subjected to functional ELISA assays to test binding to the spike protein receptor binding domain (RBD). The functional assay begins with coating plates with target aptamer or control human ACE2 protein (FIG. 28). Biotinylated spike-RBD is then added after 3 rounds of washing and incubated to allow saturation. Bound spike-RBD is then assayed using Streptavidin-HRP conjugate followed by incubation with TMB solution and absorbance at 450 nm as the signal detection (FIG. 28A). Analysis of the collection of Gyrl-like aptamers showed that CTR Y106W and SAV HS are the most potent binding aptamers for spike-RBD (FIG. 28B). SAV HS demonstrated comparable binding to ACE2 receptor while CTR Y106W showed much higher binding. Control experiments where wells were coated with non-target antibody showed low binding to biotinylated SARS-COV-2 antibody (FIG. 28B). Wildtype CTR107 and SAV2435 showed strong binding to SARS-COV-2 spike protein as expected. The promiscuous binding nature of Gyrl-like proteins can allow for wildtype protein binding to targets with moderate affinity. The therapeutic applications of wildtype proteins will be limited because of promiscuous binding to various non-target molecules. SAV HS and CTR Y106W are candidates for further exploration for biopharmaceutical functions. These results demonstrate the first example of engineered Gyrl-like proteins binding to SARS-COV-2. Furthermore, these results confirm the proof-of-principle that Gyrl-like aptamers can be engineered to recognize protein targets

### Example 16: Structural Features of Spike-Binding Gyrl-Like Aptamers

Since engineered SAV HS and CTR Y106W show strong binding to spike-RBD, they were chosen as the prototype for developing therapeutic Gyrl-like aptamers. Using both templates, the inhibitory effects of Gyrl-like aptamers on SARS-COV-2 spike protein can be screened and further refined to improve function. The structural designs, protocols, tools and methods used herein can be utilized to develop new and improved aptamers that can bind and inhibit the SARS-COV-2 spike protein.

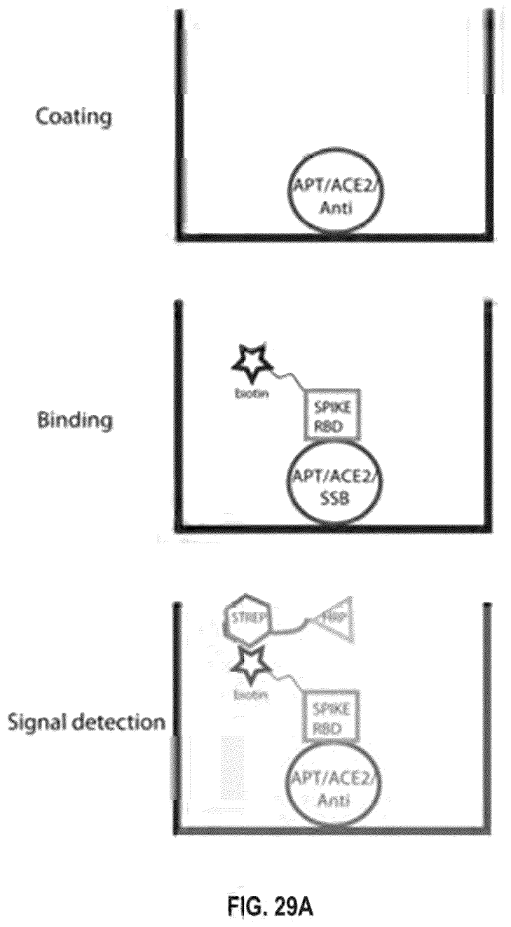

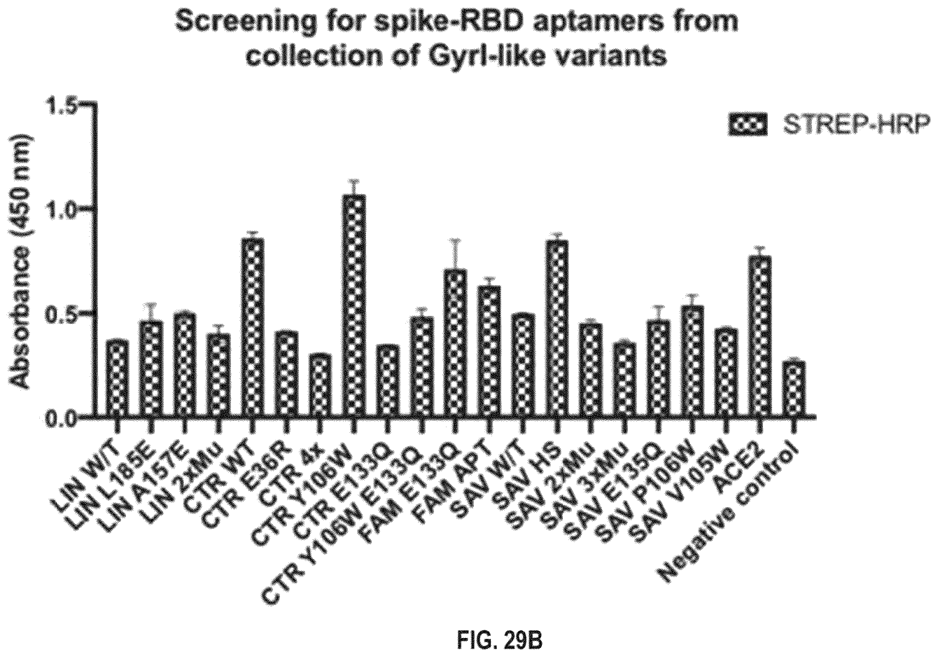

CTR Y106W represents an aptamer with partial specificity because it also has the ability to recognize other molecules such as Daunorubicin. CTR Y106W was rationally designed to remove a flexible tyrosine at amino acid position 106 (numbering according to crystal structure) with a tryptophan residue with the goal of reducing conformational plasticity within the binding site (FIG. 29B). This substitution increases aromaticity and hydrophobicity, in addition to expanding the binding cavity to tolerate the larger tryptophan residues. This single mutation is sufficient for altering the properties of the CTR107 binding cavity that novel ligands can be bound with higher affinity. This versatile aptamer is a key prototypical rationally designed mutant that can be further engineered to improve binding specificity and function through structure-based design.

SAV HS is obtained from phage display and selection experiments using SARS-COV-2-RBD as the immobilized target. The large variant library created from NNK mutagenesis of 13 amino acids within the SAV2435 binding cavity represents a collection of predisposed aptamers that can bind to numerous target molecules (FIG. 29A). The high stringency of the phage display employed and selection protocol ensures saturation of high affinity aptamers in biopanning experiments, as opposed to previous efforts without high stringency resulted in high diversity of aptamers, many of which formed insoluble aggregates during purification. The inclusion of 10-fold higher detergent in wash buffer and the inclusion of 8 rounds of stringent biopanning resulted in complete saturation of a single aptamer. Analysis of 10 colonies in plaque assay showed that each unique plaque contains the same sequence identity, suggested that a single high affinity phage was enriched in process. Alignment of SAV HS with wildtype SAV2435 showed that a total of 13 unique mutations are present in the binding site. Mutations are present at amino acid positions 27, 30, 31, 34, 38, 89, 105, 106, 109, 110, 113, 135, 137 (numbering according to crystal structure). The identity of each mutation is listed in FIGS. 16A and C.

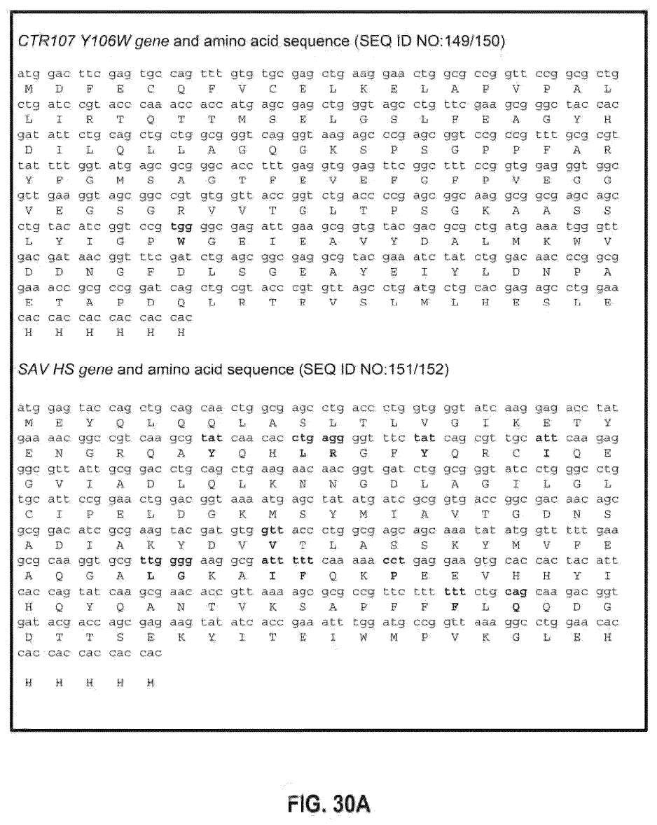

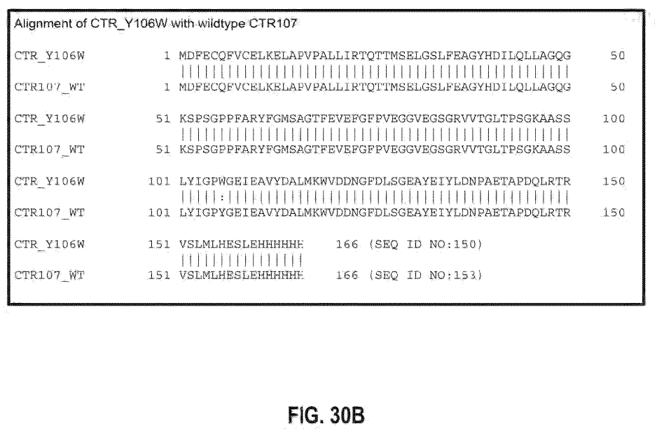

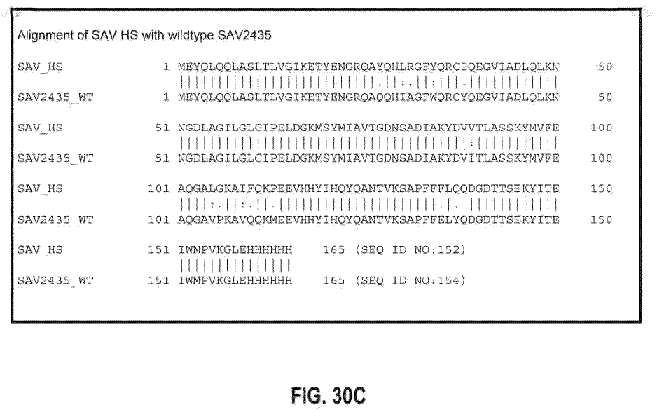

To understand the structural changes that occur in SAV HS, a homology model was constructed using the crystal structure of SAV2345 bound to the rhodamine 6G (5KAU). Alignment of the homology model to the wildtype crystal structures shows drastic changes in the binding site induced by mutagenesis (FIG. 30). Notably, the binding site is more expansive in size and the physiochemical properties of discrete binding surfaces is different. E135 is a conserved residue in most Gyrl-like protein and is important for function in some instances. In SAV HS this conserved residue is mutated to a phenylalanine residue resulting in a large hydrophobic change. In the vicinity of this phenylalanine substitution are two aromatic substitutions, Q110F and Q27Y which creates a novel hydrophobic surface which is likely important in spike-RBD epitope binding. Towards the more surface exposed regions of the pocket several aromatic residues are mutated to polar residues. The Y137Q and W34Y mutations change the accessibility of the binding pocket and create polar binding regions that can likely form hydrogen bond with spike-RBD epitopes. Overall, this invention demonstrates a method to reorganize and modify the SAV 2435 binding pocket for better physical and chemical complementarity to target binding surfaces. With this technology, further engineering can be conducted to improve binding specificity. The crystal structure of SAV HS in complex with spike-RBD would provide the necessary framework for further structure-guided engineering.

### Example 17: Comparison of the Binding by Spike-RBD Gyrl-Like Aptamers to ACE2 and SARS-COV-2 Antibody

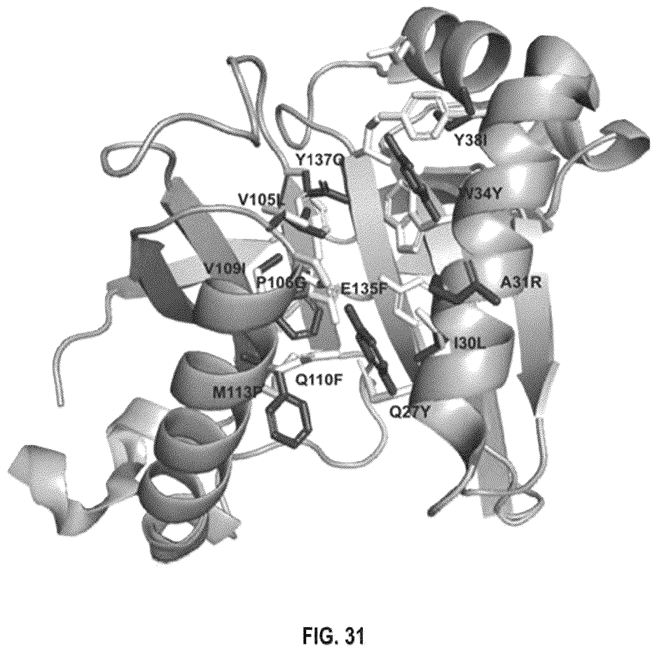

To further confirm high affinity spike-RBD binding by Gyrl-like proteins the protocol described in FIG. 15A was used to compare spike-RBD binding to human ACE2 and SARS-COV-2 anti-spike protein antibody. Human ACE2 and SARS-COV-2 antibody have been shown to bind to spike-RBD with 34 nM and 5 nM affinity, respectively. Comparable binding by Gyrl-like aptamers will confirm nM affinity and provide strong evidence that affinity is within range for therapeutic use. In this experiment, coating was done with either aptamer, human ACE2 or SARS-COV-2 antibody, and either biotinylated spike-RBD or spike-RBD-Fc was used for binding, which can be detected with streptavidin-HRP or goat-anti-mouse (GAM)-HRP (FIG. 31). Using either method for signal detection provided data that SAV HS and CTR Y106W can bind spike-RBD with comparable or higher affinity than ACE2 (FIG. 18). The binding observed was weaker than the antibody but strong enough that it may induce the same inhibitory effects. The stronger binding signal observed with GAM-HRP could be due to nonspecific binding effects between Ig domain and proteins used in experiments. These results confirm that the invention Proteimers can bind spike-RBD with nanomolar affinity within the range required for therapeutic effects.

### Example 18: Gyrl-Like Aptamers' Inhibition of Spike-RBD Interactions with ACE2 and Anti-Spike Protein Antibody

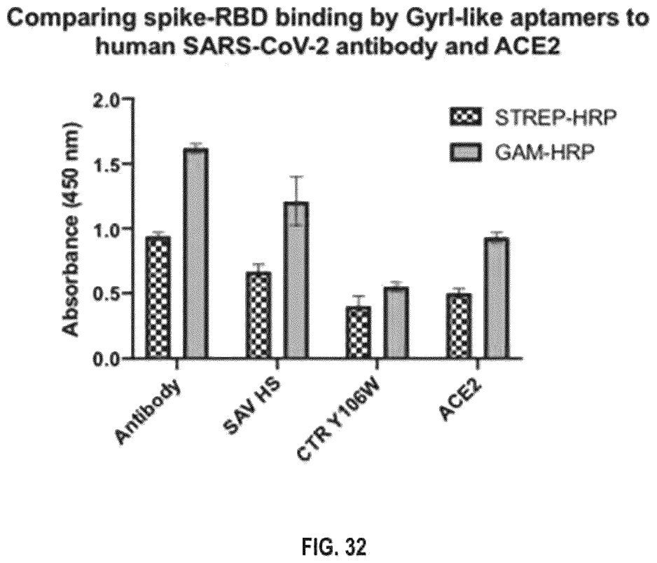

For Gyrl-like proteins to be used as therapeutic proteins, they should exhibit potent inhibition of SARS-COV-2. In the structure-based designs workflow of the invention, aptamers are contemplated to inhibit viral entry by two mechanisms. The first is through competitive inhibition where an aptamer binding to SARS-COV-2 blocks viral binding to ACE2 receptor on host cells. In the second mechanism noncompetitive inhibition occurs when an aptamer binds to SARS-COV-2 and induces membrane shedding or binds at a site that allosterically lock spike protein in an inactive conformation without disrupting ACE2 binding. In the competitive mechanism, aptamers function like neutralizing antibodies and must bind to the same surface or epitopes that interacts with ACE2 receptor. In a non-competitive mechanism, aptamers can bind to distinct regions of Spike-RBD without affect ACE2 binding. In this instance, a spike-RBD-aptamer-ACE2 complex can assemble, but would be inactive because of allosteric inhibition or protease cleavage inhibition. In FIG. 32A a schematic representation of an inhibitory ELISA screen is provided for studying the mechanism of inhibition. In this assay, the inhibitory effects of aptamers can be studied by immobilizing spike-RBD-antibody or spike-RBD-ACE2 complex on plates and monitoring whether the addition of Gyrl-like aptamers can disrupt binding (FIG. 32A). In these experiments either biotinylated spike-RBD or spike-RBD-Fc can be immobilized with an antibody or ACE2. In experiments to test the inhibition of spike-RBD-ACE2, negative control experiments containing no inhibitor and only the spike-RBD-ACE2 complex is present. The SARS-COV-2 anti-spike protein antibody is used as a positive control to demonstrate ACE2 inhibition. At equimolar concentrations, SAV HS and CTR Y106W completely inhibited ACE2 binding as much as control experiments with antibodies (FIG. 32B). This finding indicates that the invention Proteimers SAV HS and CTR Y106W can inhibit spike-RBD-ACE2 interactions as efficiently as neutralizing antibodies. Furthermore, the results of these inhibitory ELISA assays strongly suggest that aptamers can block spike-RBD-ACE2 interaction through a competitive mechanism. To further validate the inhibition, similar experiments were conducted where Gyrl-like aptamers were tested for blocking spike-RBD-antibody interactions. The interactions between the spike protein and anti-SARS-COV-2 antibody are 2-5 fold stronger than ACE2. Disruption of this complex by Gyrl-like aptamers would indicate high affinity binding to spike-RBD, further supporting their use as therapeutics. Using GAM-HRP for signal detection it is evident that SAV HS and CTR Y106W did inhibit spike-RBD binding to the anti-spike-RBD antibody with similar potency exhibited in experiments with spike-RBD-ACE2 (FIG. 32C). Overall, these results confirm that the invention Proteimers SAV HS and CTR Y106W can strongly bind to SARS-COV2 and inhibit binding through a competitive mechanism. This competitive inhibition can be further modified to create Gyrl-like aptamer therapeutics for treating SARS-COV-2.

### Example 19: Additional Proof of the Inhibition of Spike-RBD ACE2 Complex by Gryl-Like Aptamers

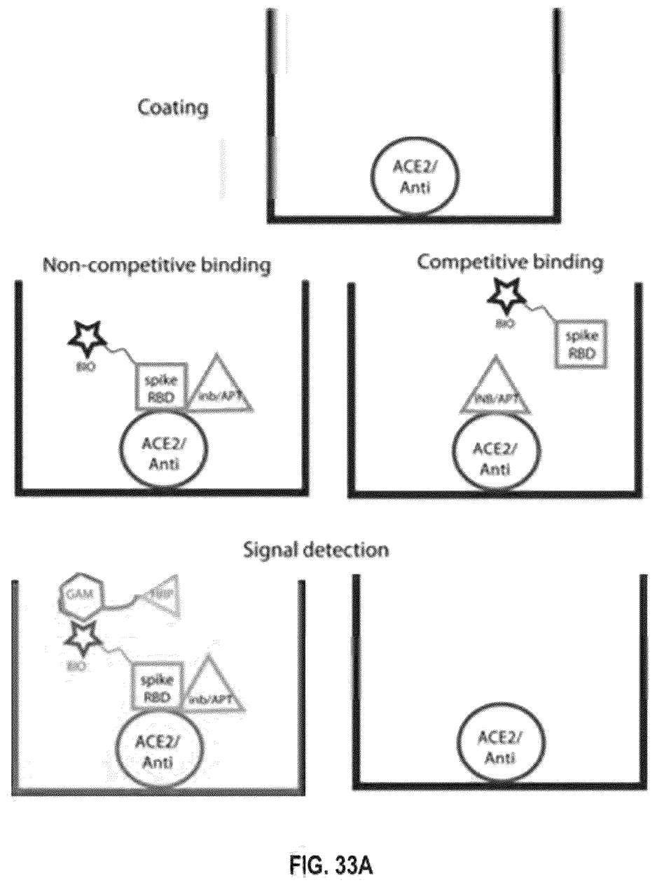

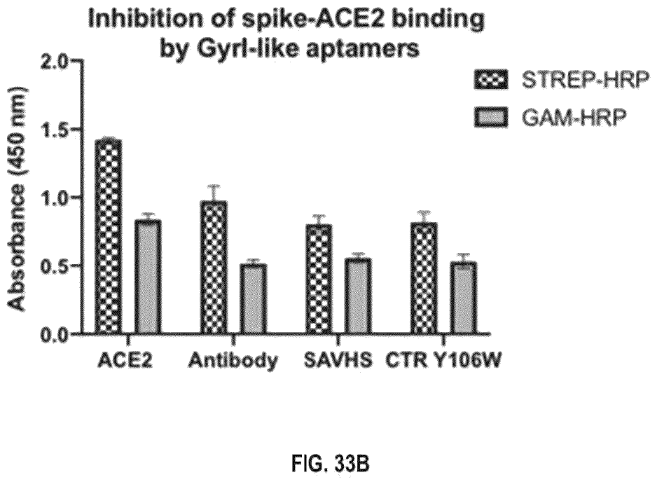

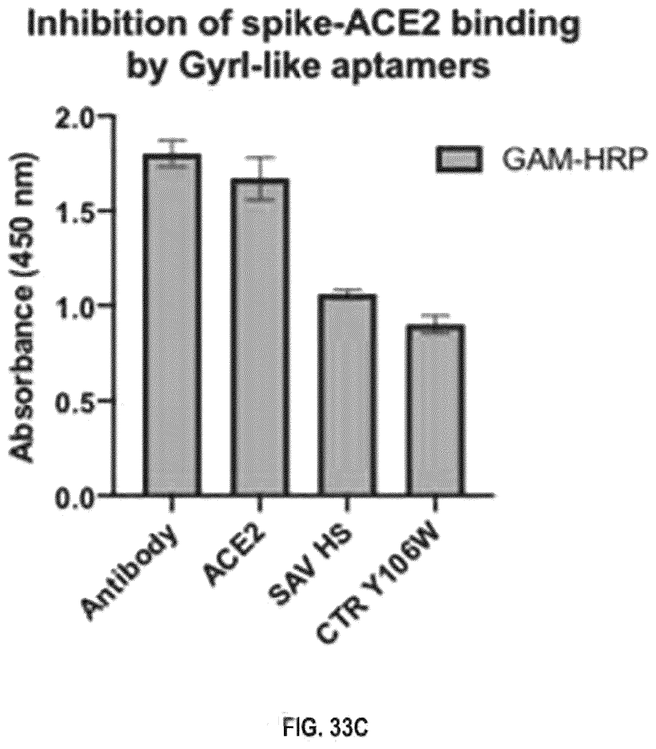

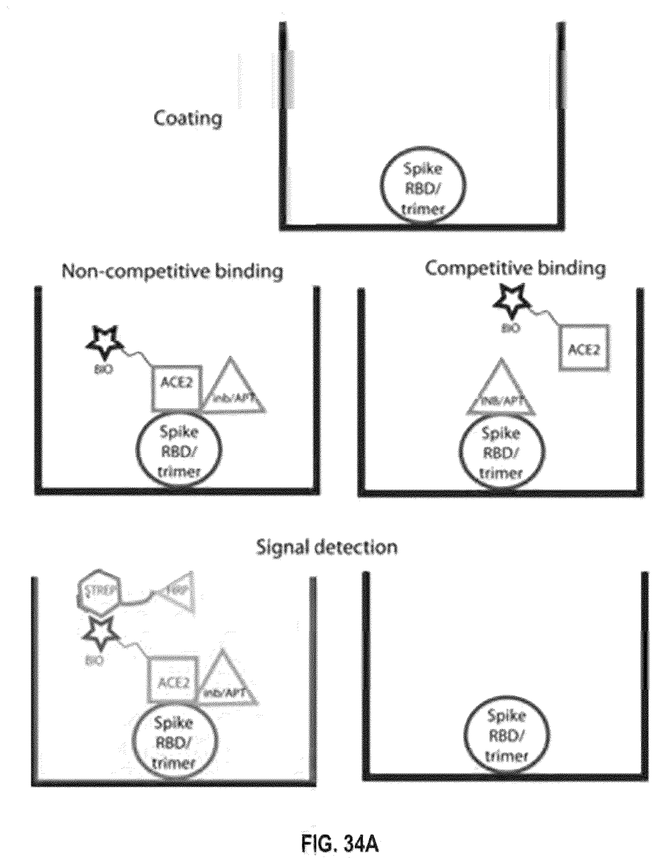

To confirm inhibitory effects additional methods were developed to screen for Spike-RBD-inhibition. The inhibitory ELISA was redesigned by coating plates with untagged Spike-RBD and signal detection obtained through biotinylated ACE-2 binding to Streptavidin-HRP (FIG. 33A). This experiment is complementary to FIGS. 19A and 19B and further confirms the inhibition of spike-RBD-ACE2 interaction by Gyrl-like aptamers. In these experiments, coating with spike-RBD produced similar results as coating with ACE2. The inhibition by SAV HS and CTR Y106W were similar and comparable to the inhibition by anti-spike-RBD antibody (FIG. 33B). The results presented here along with those in example 14 confirm the inhibition of spike-RBD by Gryl-like aptamers.

To further demonstrate the therapeutic properties of Gyrl-like aptamers, additional ELISA methods were developed where the physiological spike trimer was used for coating ELISA plates. In these experiments, the complex between ACE2 and the functional spike trimer can be reconstituted and immobilized on ELISA plates (FIG. 20A). Gyrl-like aptamers can then be tested for inhibitory effects on the functional complex used by SARS-COV-2 for host cell entry. Coating with the spike trimer produced a stable complex with biotinylated ACE2 but the overall signal was weaker compared to the immobilized spike-RBD-ACE2-biotin (FIG. 33C). When SAV HS and CTR Y106W are added, inhibition that was comparable to when anti-spike protein antibody was added is observed (FIG. 33C). These results confirm that Gryl-like aptamers can inhibit binding of the full-length spike trimer binding to ACE2 receptor. Inhibition of the physiological complex used for host cell entry is a fundamental proof of the therapeutic potential of Gyrl-like aptamers.

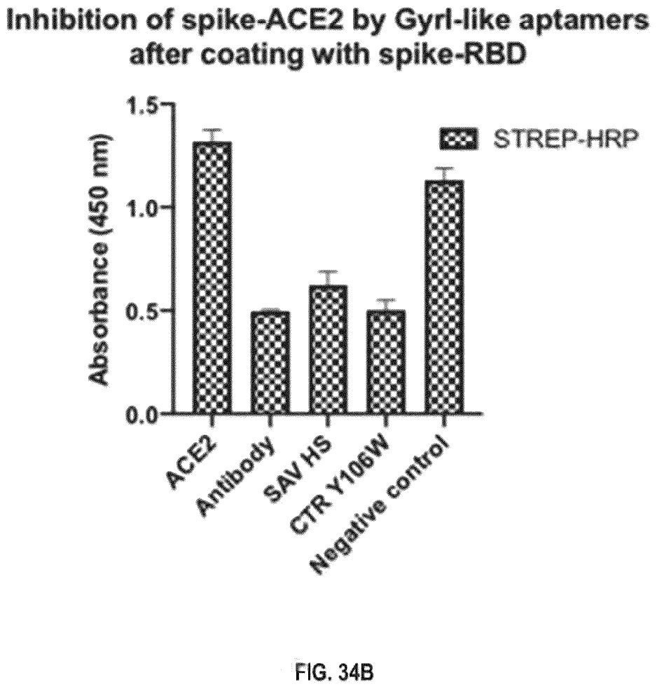

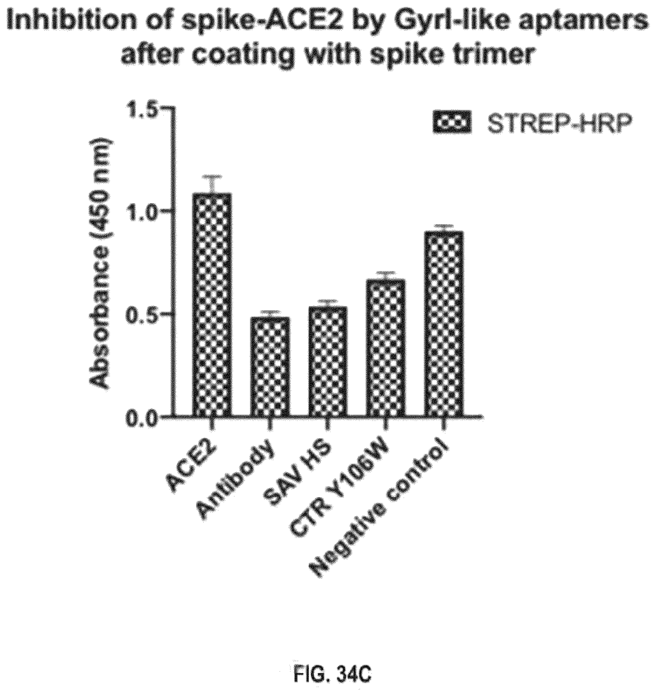

In addition, a dose-dependent inhibition of the spike-RBD-ACE2-biotin complex by Gyrl-like proteins is demonstrated using this protocol. Immobilized spike-RBD-ACE2-biotin incubated with increasing concentration of SAV HS, CTR Y106W or anti-spike protein antibody shows a dose-response inhibition. SAV HS achieved maximal inhibition at concentrations as low as 2 nM (FIG. 34). The inhibition profile for SAV HS is similar to the antibody which highlights that spike binding is high affinity. CTR Y106W required a 15 nM to achieve full inhibition. From this data it is evident that SAV HS is a more potent inhibitor than CTR Y106W. Overall, SAV HS displays high binding to spike proteins or stronger inhibition of the ACE2 complexes in all experiments conducted. This is because SAV HS was obtained from stringent biopanning and selection experiments, while CTR Y106W was rationally designed to improve binding. The similar dose-response inhibition profile to SARS-COV-2 anti-spike protein antibody shows that SAV HS can potently inhibit spike-RBD-ACE2 complex. These inhibitory effects can be further refined to create therapeutic aptamers for treating patients infected with SARS-COV-2. Such technology can be used as a cheaper and more effective replacement of convalescence antibody therapy.

### Example 20: a Structure Prediction of SARS-COV-2 Spike-RBD Binding Proteimer of SAV-HS

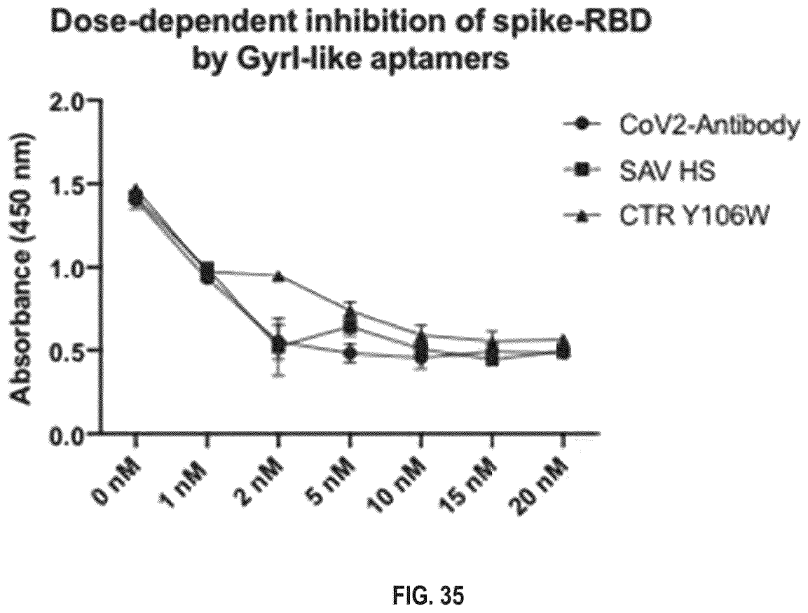

To understand the structural effects of site-directed point mutations on SAV-HS, AlphaFold2 was utilized for structural predictions and visualizing those results on ChimeraX (FIG. 35). AlphaFold2, an AI program developed by DeepMind, was employed due to its exceptional performance and accuracy in protein structure prediction. Two-thirds of the AlphaFold2 predictions were comparable in quality to experimentally derived structures. To visualize these predictions, ChimeraX, a molecular visualization program created by UCSF, was used due to the several user-friendly features such as high-resolution imagery, virtual reality rendering, comparative overlay and molecular analysis interactions.

These tools provide assist in understanding how specific mutations on a Proteimer enable high affinity and specificity. AlphaFold2 predicted structures reveal drastic changes in the binding site induced by mutagenesis (FIG. 35). Notably, the mutations in the binding pocket of the protein resulted in an enlargement of the pocket, making it more spacious in size. Additionally, the physiochemical properties of the individual binding surfaces within the pocket have undergone changes, leading to distinct characteristics and properties compared to the original binding site (FIG. 35B). E135, a conserved residue in most Proteimer proteins, is substituted for a phenylalanine residue in SAV-HS, which causes a significant hydrophobic alteration. In close structural proximity to this phenylalanine mutation, two aromatic substitutions, Q110F and Q27Y, create a new hydrophobic surface that is likely essential for spike-RBD epitope binding.

Furthermore, a few aromatic residues are substituted with polar residues in the more externally exposed areas of the pocket (FIG. 35B). The Y137Q and W34Y mutations drastically change the accessibility of the binding pocket, creating polar binding regions that could likely form hydrogen bonds with spike-RBD epitopes. Overall, this invention demonstrates a method to reorganize and modify the SAV2435 binding pocket, and like binding pockets, for enhanced physical and chemical complementarity to target binding surfaces. Further engineering is contemplated to improve binding specificity. The modeled structure of SAV-HS in complex with spike-RBD provides the necessary framework for further structure-guided engineering.

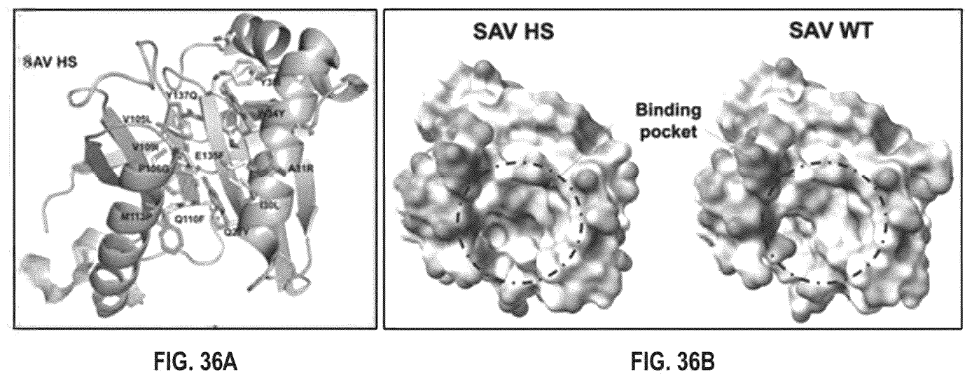

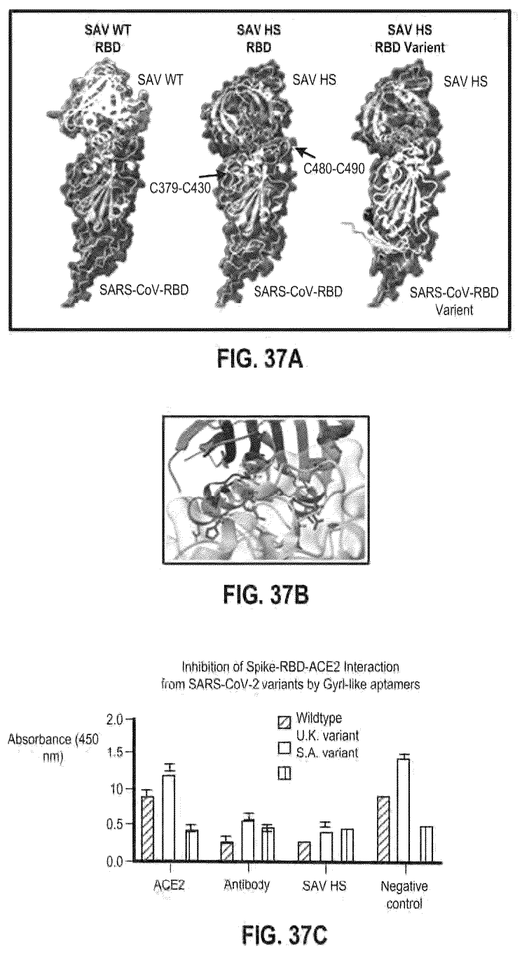

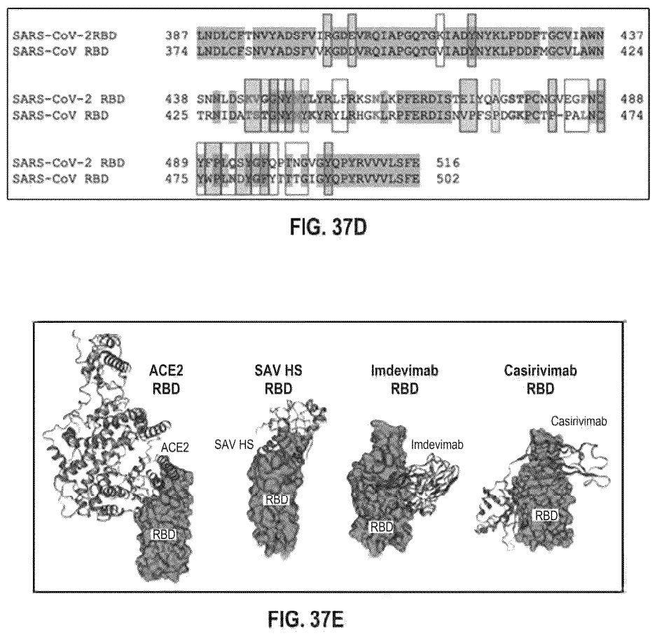

AlphaFold2 and HDOCK was used to model the predicted protein-protein interaction of Proteimer SAV-HS with SARS-COV-2-RBD. Preliminary structural analysis revealed that the residues essential for SAV-HS binding in the SARS-COV-2 receptor-binding domain (RBD) are similar to the residues critical for ACE2 binding. Furthermore, a majority of these residues are highly conserved or possess similar side chain properties when compared to the corresponding residues in the SARS-COV-2-RBD. In comparison to SAV WT (wild-type), SAV-HS exhibits a significant increase in interactions with the SARS-COV-2-RBD. SAV-HS exhibits extensive binding with the SARS-COV-2-RBD, as evidenced by 20 residues establishing contact in FIG. 23A. Among these residues, N22, R24, P63, E64, L65, D66, G67, G92, S93, E114, E115, H117, H118, H121, Q122, N126, K129, S130, A131, and K156 form a binding interface with the SARS-COV-2-RBD, with an atomic distance of 3.5 A (FIG. 36B). This indicates a significant and intricate interaction between the two proteins. When employing a distance cut-off of 3.5, a total of 33 RBD residues were identified that are in contact with 20 SAV-HS residues, while a similar total of 27 RBD residues are in contact with 23 residues of ACE2 (FIG. 36D). Furthermore, it has been observed that the variants of the SARS-COV-2 receptor-binding domain (RBD), such as Alpha, Beta, Gamma, and Delta, do not exhibit significant modifications in the binding interface (FIG. 36A). In addition, SAV-HS demonstrates effective binding affinity against both the SARS-COV-2 RBD UK and SARS-COV-2 RBD SA variants, in comparison to the wild-type SARS-COV RBD (FIG. 236C). As set forth herein, SAV-HS specifically targets a conserved region in the spike RBD protein (FIG. 36D). The regions on the spike RBD protein where SAV-HS and ACE2 bind were mapped and compared them to currently available antibodies (Imdevimab, Casirivimab) on the market (FIG. 36E). In most cases, SAV-HS did not overlap with the majority of monoclonal antibodies (FIG. 36E).

What is claimed:

1. A non-native variant proteimer, which is selected from:
a non-native variant proteimer, relative to wild-type TEX264, comprising any combination of one up to all 8 of variant amino acid residues, wherein the variant amino acid residues correspond to residues 18, 19, 25, 26, 29, 102, 105 and 106 of TEX-S2 (SEQ ID NO:2);
a non-native variant proteimer, relative to wild-type TEX264, comprising any combination of one up to all 12 of variant amino acid residues, wherein the variant amino acid residues correspond to residues 18, 19, 22, 25, 26, 29, 40, 46, 102, 105, 106, and 136 of TEX-S3 (SEQ ID NO:4);
a non-native variant proteimer, relative to wild-type TEX264, comprising any combination of one up to all 12 of variant amino acid residues, wherein the variant amino acid residues correspond to residues 31, 32, 35, 38, 39, 42, 53, 59, 115, 118, 119, and 149 of TEX-S4 (SEQ ID NO:6);
a non-native variant proteimer, relative to wild-type YTHDF3, comprising any combination of one up to all 11 of variant amino acid residues, wherein the variant amino acid residues correspond to residues 40, 54, 55, 57, 84, 108, 113, 129, 147, 149, and 150 of YTHDF3 (SEQ ID NO:8);
a non-native variant proteimer, relative to wild-type PUM, comprising any combination of one up to all 24 of variant amino acid residues, wherein the variant amino acid residues correspond to residues 37, 38, 41, 73, 74, 77, 109, 110, 113, 145, 146, 149, 181, 182, 185, 217, 218, 221, 253, 254, 257, 296, 297, and 300 of PUM (SEQ ID NO: 10);
a non-native variant proteimer, relative to wild-type DARPin, comprising any combination of one up to all 21 of variant amino acid residues, wherein the variant amino acid residues correspond to residues 32, 34, 35, 37, 45, 46, 58, 65, 67, 68, 70, 78, 79, 91, 98, 100, 101, 103, 111, 112, and 124 of DARPin (SEQ ID NO:12);
a non-native variant proteimer, relative to wild-type Aca2, comprising any combintion of one up to all 14 of variant amino acid residues, wherein the variant amino acid residues correspond to residues 28, 30, 31, 33, 34, 39, 45, 149, 151, 152, 154, 155, 160, and 166 of Aca2 (SEQ ID NO:14); and
a non-native variant proteimer, relative to a wild-type nanobody, comprising any combination of one up to all 22 of variant amino acid residues, wherein the variant amino acid residues correspond to residues 28, 29, 30, 31, 32, 34, 35, 37, 56, 57, 58, 61, 103, 104, 105, 106, 107, 108, 109, 110, 111, and 112 of the nanobody (SEQ ID NO: 18).

2. A chimeric proteimer construct comprising,
a proteimer moiety according to claim 1; and
a recombinant polypeptide moiety.

3. The chimeric proteimer of claim 2, wherein the polypeptide moiety is selected from one or more of a proteolytic degradation moiety, an Fc-region moiety and/or another proteimer moiety.

4. The chimeric proteimer of claim 3, wherein the proteolytic degradation moieity is selected from proteases and/or nucleases.

5. The chimeric proteimer of claim 4, wherein the nuclease moiety is an RNAse moiety selected from one or more of: RNase A, PIN, SMG6 PIN, RENT1/UPF1 PIN, viral PIN domains, RNase MRP, RelE toxin PIN, Cas13, Onconase, RNAse 2, RNAse 3, RNAse 5, Barnase, Binase and/or pinard.

6. The chimeric proteimer of claim 5, wherein the chimeric proteimer binds and cleaves a specific RNA target.

7. The chimeric proteimer of claim 6, wherein the RNA target comprises an Internal Ribosomal Entry Site (IRES) and/or an Iron-Responsive Element (IRE).

8. The chimeric proteimer of claim 6, wherein the RNA target is a 5′ UTR region of an mRNA transcript coding for a protein selected from: c-myc, APP, tau, SNCA (Alpha-synuclein), ARID5A, A323M, Ferritin, and C-jun.

9. The chimeric proteimer of claim 2, further comprising one or more of a pumilo (PUM) domain, a blood-brain barrier transcytosis domain, and a half-life extending domain.

10. The chimeric proteimer of claim 9, wherein the pumilo (PUM) domain is selected from a non-native variant proteimer, relative to wild-type PUM, wherein the blood-brain barrier transcytosis domain is selected from Angiopep-2 peptide, HIV-1 Tat peptide, Rabies Virus Glycoprotein (RVG29), RDP-shuttle peptide, and/or Melanotransferrin-derived peptides; and wherein the half-life extending domain is selected from a transferrin receptor targeting protein domain, an FcBP domain, PEGylation, XTEN, PASylation and/or an albumin binding domain.

11. The chimeric proteimer of claim 9, wherein pumilo domain is amino acids 27-376 of SEQ ID NO: 176; the blood-brain barrier transcytosis domain is Angiopep-2 peptide; the half-life extending domain is an albumin binding domain; or
wherein the proteimer moiety is selected from the group consisting of SEQ ID NOs: 16, 17, 19, 21, 22, 24, 25, 30-38, 112-115, 124-138, and 141-148; or
wherein the protease moiety is obtained from one or more of: TRIM21, VHL, CRBN, MDM2, RNF4, FBW7, CHIP, FBXW7, Subtilisin, Trypsin, TEV protease, Granzyme B, Caspases, Papain, Cathepsins, Calpain, MMPs, ADAMs, HIV protease, and/or Cathepsin D; or
wherein the polypeptide moiety is an Fc-region moiety and the proteimer moiety is selected from the group consisting of SEQ ID NOs: 27, 28, 40-110, and 116-121; o which is selected from:
TEX-CD19_8: Fc-region (SEQ ID NO:178); and
TEX-CD19_6: Fc-region (SEQ ID NO: 179).

12. A chimeric multivalent proteimer construct comprising,
a first and second non-native variant proteimer of claim 1.

13. The chimeric multivalent proteimer of claim 12, wherein the multivalent proteimer construct further comprises a half-life extending domain.

14. The chimeric multivalent proteimer of claim 13,
wherein the half-life extending domain is selected from a transferrin receptor targeting protein domain, an FcRn-binding peptide domain, Fc-region, PEGylation, XTEN, PASylation and/or an albumin binding domain;
wherein the first and second proteimer moiety binds to a protein selected from the group consisting of: CD19, CD3, CD33, CD37, Hur, CD20, CD22, Bcl2, Her2, CD37;
wherein the first and second proteimer moiety are selected from any combination of 2 or more of SEQ ID NOs: 27, 28, 47-62, 73-110, and 116-121; or
wherein the first and second proteimer moiety are selected from any combination of 2 or more of SEQ ID NOs: 27, 28, 47, 48, 55-57, 93-103 and 119, wherein the first protiemer moiety binds to CD19 and the second proteimer moiety binds to CD20.

15. A method of treating cancer, comprising administering to a patient in need thereof, a chimeric proteimer construct of claim 2, wherein the proteimer moiety is selected from one or more of SEQ ID NOs: 27, 28, 47-62, 73-110, 116-121, 131-138, and 141-148.

16. The method claim 15,
wherein the proteimer moiety binds to a protein selected from the group consisting of: CD19, CD3, CD33, CD37, Hur, CD20, CD22, Bcl2, Her2, CD37;
wherein the proteimer moiety binds to CD19;
wherein the proteimer moiety is SEQ ID NO: 27 and/or SEQ ID NO:28,
wherein the chimeric proteimer construct is SEQ ID NO: 178 (CD19_8-Fc) and/or SEQ ID NO: 179 (CD19_6-Fc),
wherein the proteimer moiety binds to a 5′ UTR mRNA target coding for a protein selected from the group consisting of: cMyc-A323M and ARID5A,
wherein the proteimer moiety is selected from one or more of SEQ ID NOs: 131-138 and 141-148.

17. A method of treating Alzheimer's disease, comprising administering to a patient in need thereof, a chimeric proteimer construct of claim 2, wherein the proteimer moiety is selected from one or more of SEQ ID NOs: 21, 22, 24, 25, 32-38, 63, 64, 113-115, and 124-130.

18. The method claim 17,
wherein the proteimer moiety binds to a 5′ UTR mRNA target coding for a protein selected from the group consisting of: APP, Tau, Ferritin, and Tau60;
wherein the proteimer moiety binds to a 5′ UTR mRNA target coding for APP;
wherein the proteimer moiety is SEQ ID NO: 21 and/or SEQ ID NO:22;
wherein the chimeric proteimer construct is SEQ ID NO: 176 (ProAPPS3-5) and/or SEQ ID NO: 177 (ProAPPS3-11);
wherein the proteimer moiety binds to a 5′ UTR mRNA target coding for Tau,
wherein the proteimer moiety is SEQ ID NO: 24; or
wherein the chimeric proteimer construct is SEQ ID NO: 180 (ProTEX-T1).

19. A method of treating Parkinson's disease, comprising administering to a patient in need thereof, a chimeric proteimer construct of claim 2, wherein the proteimer moiety is selected from one or more of SEQ ID NOs: 16, 17, 19, 30, 31, and 112.

20. The method claim 19, wherein the proteimer moiety binds to a 5′ UTR mRNA target coding for alpha-Synuclein.
